SEQUENCE LISTING (150 sequences)

SEQ ID NO: 1 - DNA, 2498 bp, Mus sp.
gccggaaggagtgtgggagacagttggttgctctggtttctcctggcggcggcgttagcgacgaagctgcctttggcagcatgaacccgctgtgaggcgcaggcttcgcggcgcggggggcggggggaagaggaggacgcctcggcgaagttctccgccatgaacctgaggggcctctttcaggacttcaacccgagtaaattcctcatctatgcctgtctgctgctgttctctgtgctactggcccttcgtttggatggcatcattcaatggagctactgggctgtctttgctccaatatggttgtggaagttaatggtcattgttggcgcatcagttggaactggagtttgggcacgaaatcctcagtatcgagcagaaggagaaacatgtgtggagtttaaagccatgttaattgctgtgggcatccatttgctcctgctgatgtttgaagttctggtctgtgacaggattgagagaggaagccatttctggcttctggtcttcatgccattgttctttgtttccccagtgtctgttgcagcatgtgtctggggctttcgacatgacaggtcactagagctagaaatcctgtgttcggtcaacattcttcagtttatattcattgccttaagactggacaagattatccactggccctggcttgttgtgtgtgtccctctgtggattctcatgtccttcttgtgcctggtggtcctctattacatcgtttggtcggttctgttcctgcgctctatggatgtgattgcagaacaacgcagaacacatataaccatggcactgagctggatgaccattgtcgtgcctcttcttacttttgagatcctgctggttcacaaactggatggccacaatgctttctcctgtattccaatatttgttcccctgtggctctcattgatcacactgatggcaacaacatttggacagaaaggaggaaaccactggtggtttggtattcgcaaagacttctgccagtttctgcttgaaatattcccgtttttgagagagtatggaaacatttcatatgatctccaccatgaagatagtgaggaaacagaagaaaccccagttccagaacctcctaaaattgcacctatgttccgcaagaaggccagggtagtcatcacccagagccctggcaagtatgtccttcctccacccaaactaaatattgaaatgccagactaaaggacgccaccagggccgccggagtgtgtgtgcactgagacacacactcctacttcccttgcccccaacctagacctggaacctcggaccaccatctgaagcctcgtttgcatccagtgtcaaccagccactcagaacagaacataggtggcagggactaagggtatcttatcaatgtggatgccagtgacacatgagcaaagaacctatttctttcgtatgggtacctgaaatgtgttctcattcctgtgatacccacccagggatgactgctgcctaggcccacagaaagcacatctgtggtctgggcagattttctagtgctaaaattcctatcaaagtattgatgatgtggtcctctggacacttcatctgtgctaactggctgctatggtagcagaaggactataagaggtctgacacactgtgtgtcagatcagccggccctgcttccttgtctctgctgacagaaattagcttagccaggatgacagcagggttgcagggcctcatcagtgagcccttcccaatggcatgattccatgcctatatagtattatacagctattttagcactgtaatatacagtgtcaaatcctagttctgtacagcatattctctgttaaagtatttttttacaagcttgtgctggagatggatgggttaagccatgggagaagcagatagctcagactgctgtatctcagtcagatgccccacctcacttaccatgcccactgataccctggaatttctgccagcacagctactacaatcaaggaaagctctgtgcttttccaacatggggtgtgggagaaatggcttaaaagcccaagaaatgcccccttgagaccaggacactagagaaggttgccaaggcaggccagaatcagccaggaactcctgcaaagtcgagagcattctgcctacctgcaagaagaacactgcatactgggcatgcaggtaccaccacctgtcagcagtttctccaggctctgctggtatagccagcccagaacccatcataccagaaaataaccttcagtctaaatcaaatgattcattcatggtcacctgatcagtggcttcagtcacacataattgaagtctctaaccctagtcctacccttggcacaggcctccagtgagcctgttctgtttcagaagaaaggggaagcagtgtagcattttttgtttggtctcttaaaaatgtggttaaatccactattttttgctcctgtagttcaataaagtggttcattttacatgtaaaaaaaaaaaaaaaa

SEQ ID NO: 2 - DNA, 456 bp, Mus sp.
acaagcttgtgctggagatggatgggttaagccatgggagaagcagatagctcagactgctgtatctcagtcagatgccccacctcacttaccatgcccactgataccctggaatttctgccagcacagctactacaatcaaggaaagctctgtgcttttccaacatggggtgtgggagaaatggcttaaaagcccaagaaatgcccccttgagaccaggacactagagaaggttgccaaggcaggccagaatcagccaggaactcctgcaaagtcgagagcattctgcctacctgcaagaagaacactgcatactgggcatgcaggtaccaccacctgtcagcagtttctccaggctctgctggtatagccagcccagaacccatcataccagaaaataaccttcagtctaaatcaaatgattcattcatggtcacctgatcagtggcttcagtc

SEQ ID NO: 3 - DNA, 2034 bp, Mus sp.
gtcgccgagtccgcagcgcgccaggtcccatccgactccgcacgcaacgcggcgtcggcctcgtggctgcgaaatgaaggcagacggcgtgagcatccactgaagcctgagctacggggttggggcagtgtccccggcgtaggcaccgtaccgttcgcggccagcgcacgctgtgccctcggctcgcttccggccgggccacgtgactgctcgcacgtgttccggcccctcggagctgcggtctggagcctctaacacgcagcgggcggatgcccatctagaggcagtacagccttcgccatgaaccccaggggcctgttccaggacttcaaccccagtaagttcctcatttatgcctgcttgctactcttctccgtgctgctgcccctgcgcctggacggcatcatccagtggagctactgggcagtcttcgcccccatctggctgtggaagctcctggtcatcgtgggcgcctcggtgggtgcgggcgtgtgggcccgcaacccacgctaccgtacagagggggaagcctgcgtggaattcaaagccatgcttatcgccgtgggcatccacctgctactactcatgtttgagatactagtctgcgacagggtggagaggggcacccacttatggctgctggtcttcatgccactcttcttcgtttcccctgtgtccgtggccgcctgtgtctggggcttccgacacgacaggtctttggaactggagatcctgtgctctgtcaacatcctgcagttcatcttcatcgccctgaggctggacaggattatccactggccgtggctagtggtgttcgtgcccctgtggatcctcatgtcattcctctgcctggtagttctctattacatcgtgtggtccctcctattcctgcggtccctggatgtggttgcagagcagcgaagaacacatgtgaccatggccatcagctggatcaccattgtcgtgcccctgctcatttttgaggtcctgctagttcacagactggatgaccacaatacgttctcttacatttccattttcatcccgctttggctctcattactgactttgatggctacaacgttcaggcggaaagggggcaatcattggtggtttgggattcgcagggatttctgtcagtttctactagaagtttttccgtttttaagagaatatggaaatatctcttacgatctccatcacgaagacagtgaagatgctgaagacgcatcggtgtcagaagctccgaaaattgctccaatgtttggaaagaaggcgcgggtagttataacacagagccctgggaaatatgtgcctccacctcccaagttaaacattgatatgccagactaaactcacaggctgggcctttcagtgaaataagaaccatatacccttgaattccactttgcttttttcttgtgttcatccagtcctggaattggaaacaggtccccctcattttacatccacactgagacagaagtctcttgcagtggtcattgtgtgccgtaggcagaagggggaaactgtaggactgtgggatgtgtacataatgtgcaagagaacttgctttccaggcccacatcttgtgagctagccaaaggtggtaaatcgaattgtttctaggccttcaaaagacctgaaaactacatagctgtttgttttaaagtgctacttatgcctaccaaaagtattgtttaaaaagtatttttatacactgctagtctaaaattgtattttagattgcacccattgtgacaaaatggcaaatgtaccaaattgtcccctcccactgttccctaaacctcattgttggtgtttctagtgctcctactgttcaaatagttgttttctgtgggctttgcggacacaggtctctagtattctaatagatgaattttctaatggagtgaatctgcatatattagtatttatatgaatgttttagcagtgttatctgtgttgattgtagttcttggcagtaatgtattgtgttaaagtatttttttttaaaaaaaaaaaaaaaaaaaa

SEQ ID NO: 4 - DNA, 2601 bp, Homo sapiens
gcagcagcgtggacgcggctggcgctggcgccatgaacccgctgtaaggcgcaggctgtgcagcacggggtgcgggggaggaggaggaggaggacgccgcggtgaagttctccgccatgaacctgaggggcctcttccaggacttcaacccgagtaaattcctcatctatgcctgtctgctgctgttctctgtgctgctggcccttcgtttggatggcatcatacagtggagttactgggctgtctttgctccaatatggctgtggaagttaatggtcattgttggagcctcagttggaactggagtctgggcacgaaatcctcaatatcgagcagaaggagaaacgtgtgtggagtttaaagccatgttgattgcagtgggcatccacttgctcttgttgatgtttgaagttctggtctgtgacagaatcgagagaggaagccatttctggctcctggtcttcatgccgctgttctttgtttccccggtgtctgttgcagcttgcgtttggggctttcgacatgacaggtcactagagttagaaatcctgtgttctgtcaacattctccagtttatattcattgccttaagactggacaagatcatccactggccctggcttgttgtgtgtgtcccgctgtggattctcatgtcctttctgtgcctggtggtcctctactacattgtgtggtccgtcttgttcttgcgctctatggatgtgattgcggagcagcgcaggacacacataaccatggccctgagctggatgaccatcgtcgtgccccttcttacatttgagattctgctggttcacaaactggatggccacaacgccttctcctgcatcccgatctttgtccccctttggctctcgttgatcacgctgatggcaaccacatttggacagaagggaggaaaccactggtggtttggtatccgcaaagatttctgtcagtttctgcttgaaatcttcccatttctacgagaatatggaaacatttcctatgatctccatcacgaagataatgaagaaaccgaagagaccccagttccggagccccctaaaatcgcacccatgtttcgaaagaaggccagggtggtcattacccagagccctgggaagtatgtgctcccacctcccaaattaaatatcgaaatgccagattagatgccacttccggggacagagcttaagtggactgggacgcactctctccgccttcctctgccccctcgttcaccccgcagaccagaaccagtactggagctgggtctccaggtacgtccatctcatgccttgtttgcatccagcgcctatcagccactcaccacgacgggacgcggaagtggcaggtgacgggggtgtgtgccagcagatgcggatgccaggaagagtgtgagaacaggggtgggattaccgtctgtctgggaggggctccaggtacccctcttccccgtcagacccactgggagatggctgcttgccaggcccccagaaggaacatctgtctatacggtgctgaaatcccaatcaaaagtattgtttagaaatgtatttctccacagggctgacctcctgcagctcgctgagcactcccaggtcctcagcactcccaggtcgtggctggggcagtcagtaggaactgtaactatgtctctgatgcaccacgtgtttagacacagcacagtccttttttctgttcctactgtggaagtagtttctctttgggcatgctgacagcagtttttcatagcctcacggatgagccctttctacgggagtgactccatgcttgtatacagagtatttatacaaatgttttagcatcttcatatgcggtgttaacccctagttctgtacagcatattctgttcaagtatttttttacaatattctgttcaagtdtttttttacaagcttgtgctgtaggcacatgccttctgccctggaactcgtgcaggtacgtagtagctgctactgccacaacggcaacaccaagcaagagatggtccatgcttttctgacgttctcagaatagtggctagcttcaaacctgacaagcgctgcttgaagccggaacactagagaatgttgctgagagcagaaacggccacgcgggtcacgactatgcgtgggaaagtctcaagcttccctcctgccagcaacaagaaggctttggagtaggcatgatgttttcacgtgtgcgtgccgtttctccaagcactgcaggttccaccgtgtgtcagaggctgcaagtttaacatcctcctgcctgaaaacaaataggtcctttgctgaaaagagggtaaaaaaagagctttgatcttctcagccaggagaagagggtggtgttttcacgcgggcaactgctcgccggcctacatggggttaattcaagtctgctgcgagcacgactccgcccttggcactggcctccagcaagccctgttctctttggggtacaggggaacgggatggtttagactttcctgctcagtgtgtaaaaaatgtagctaaagccactatttttgctctccttaagctgttcaataaaccggttcctcattttaaaaaaaaaaaaaaaaaaaaaaaaaaaaaa

SEQ ID NO: 5 - DNA, 830 bp, Homo sapiens
gcagcagcgtggacgcggctggcgctggcgccatgaacccgctgtaaggcgcaggctgtgcagcacggggtgcgggggaggaggaggaggacgccgcggtgaagttctccgccatgaacctgaggggcctcttccaggacttcaacccgagtaaattcctcatctatgcctgtctgctgctgttctctgtgctgctggcccttcgtttggatggcatcatacagtggagttactgggctgtctttgctccaatatggctgtggaagttaatggtcattgttggagcctcagttggaactggagtctgggcacgaaatcctcaatatcgagcagaaggagaaacgtgtgtggagtttaaagccatgttgattgcagtgggcatccacttgctcttgttgatgtttgaagttctggtctgtgacagaatcgagagaggaagccatttctggctcctggtcttcatgccgctgttctttgtttccccggtgtctgttgcagcttgcgtttggggctttcgacatgacaggtcactagaggtgagatttcatatatttaagaatgttttccactttgggaggtcaaggcaggtggatcacttgaggtcaggagtttgagaccagcctggccaacatggtgaaaccccatctctacttaataatacaaaaattagccgggtgtggtggcatgcgccagaatcccagcttctccggaggctgaggcgggagaatctcttaacccaggaggcggaggttgcagtgagccaagattgaaccattgcactccagcctgggtgacagaatgaaactccgtcttaaaaaaaaaaaaaaaaa

SEQ ID NO: 6 - DNA, 1065 bp, Homo sapiens
aacgctagcatggatctcgggccccaaataatgattttattttgactgatagtgacctgttcgttgcaacaaattgatgagcaatgcttttttataatgccaactttgtacaaaaaagcaggctctaccatgaacctgaggggcctcttccaggacttcaacccgagtaaattcctcatctatgcctgtctgctgctgttctctgtgctgctggcccttcgtttggatggcatcatacagtggagttactgggctgtctttgctccaatatggctgtggaagttaatggtcattgttggagcctcagttggaactggagtctgggcacgaaatcctcaatatcgttagaaatcctgtgttctgtcaacattctccagtttatattcattgccttaagactggacaagatcatccactggccctggcttgttgtgtgtgtcccgctgtggattctcatgtcctttctgtgcctggtggtcctctactacattgtgtggtccgtcttgttcttgcgctctatggatgtgattgcggagcagcgcaggacacacataaccatggccctgagctgaatgaccatcgtcgtgccccttcttacatttgagattctgctggttcacaaactggatggccacaacgccttctcctgcatcccgatctttgtccccctttggctctcgttgatcacgctgatggcaaccacatttggacagaagggaggaaaccactggtggtttggtatccgcaaagatttctgtcagtttctgcttgaaatcttcccatttctacgagaatatggaaacatttcctatgatctccatcacgaagataatgaagaaaccgaagagaccccagttccggagccccctaaaatcgcacccatgtttcgaaagaaggccagggtggtcattacccagagccctgggaagtatgtgctcccacctcccaaattaaatatcgaaatgccagattaggacccagctttcttgtacaaagttggcattataagaaagcattgcttatcaatttgttgcaacgaacaggtcactatcagtcaaaataaaa

SEQ ID NO: 7 - DNA, 1916 bp, Homo sapiens
ggcacgaggcgtccgcccaaggtcgcgggccgcttggggagtcagcagcgcgccaggccccttcgggccccacacgcattaggtgccttcttgatgggtacggagtgaacgcgggcggcggcgggaccgaggcagcgcccagtttgtaaccgccgcgccgcccgtgcccgcgcgcgccacaccccagcgcgcttccggccgggccacgtgaccgcgcgtgcacgtgttccggcctctccgcttcgccgctccgaacctcctcctggtcgtcccggcattcgtccacgcggagccggcttgggcggggcccgggaggcggcggccggagaagccgcggagacgcgagcgccgagcgtcgcgagggagcaggcccgggcaggcaagcggcggcctccgccatgaaccccaggggcctgttccaggacttcaaccccagtaagtttctcatctacacctgcctgctgctcttctcggtgctgctgcccctccgcctggacggcatcatccaatggagctactggggcgtctttgcccccatatggctgtggaagcttctagtcgtcgcaggcgcctccgtgggcgcgggcgtttgggcccgcaaccctcgctaccgcaccgagggagaggcctgtgtggagttcaaagccatgctgatcgctgtgggcatccacctgctgctgctcatgttcgaagtcctggtctgcgacagggtggagaggggcacccacttctggctgctggtcttcatgcctctcttcttcgtgtcccccgtgtccgtggctgcctgcgtctggggctttcgacacgataggtcgctggagctggagatcctgtgctcggtcaacatcctgcagttcatcttcatcgccctaaagctggacaggattattcactggccgtggctggtggtgtttgtgcccctgtggatcctcatgtcgttcctttgcctggtcgtcctctattacatcgtctggtccctcctgttcctgcggtccctggatgtggttgccgagcagcggagaacacacgtgaccatggctatcagttggataacgattgtcgtgcctctgctcacttttgaggtcctgctggttcacagattggatggccacaatacattctcctacgtctccatatttgtccccctttggctttccttactaactttaatggccacaacatttaggcgaaaggggggcaatcattggtggtttggcattcgcagagacttctgtcagtttctgcttgaaattttcccatttttaagagaatatgggaacatttcatatgatctccatcacgaagatagtgaagatgctgaagaaacatcagttccagaagctccgaaaattgctccaatatttggaaagaaggccagagtagttataacccagagccctgggaaatacgttcccccccctcccaagttaaatattgatatgccagattaaactcctagagaggacccaggcacacacagactccacctggccttcgcctcttgttcattcatcccaaacctggaaatggaaacaggcttcaaacactcgtctcacgccgtgtttgagatcaccgcctcatcagtatgcatcatagatggaggtggtttcagtatgtgggtgtgtgtggtgtgtacctgggtaagagacttgctttccaggttcgcactttcaggtgtagctgggggcagtaagtcgaattgttttagtaggtcctcaaaaggaataaccacacagctgtttgtttaaatgctactgtacctatcaaaactattgtttaaaaagtatttttatacactgctaatctaaaattgtatttcagattgtgcctgtcataacaatagcaaatgtaaaaagttctctttcccaccacttgtttataaacctcatagttgataaaaaaaaaaaaaaaaaaaa

SEQ ID NO: 8 - DNA, 2113 bp, Homo sapiens
ggcacgaggcgtccgcccaaggtcgcgggccgcttggggagtcagcagcgcgccaggccccttcgggccccacacgcattaggtgccttcttgatgggtacggagtgaacgcgggcggcggcgggaccgaggcagcgcccagtttgtaaccgccgcgccgcccgtgcccgcgcgcgccacaccccagcgcgcttccggccgggccacgtgaccgcgcgtgcacgtgttccggcctctccgcttcgccgctccgaacctcctcctggtcgtcccggcattcgtccacgcggagccggcttgggcggggcccgggaggcggcggccggagaagccgcggagacgcgagcgccgagcgtcgcgagggagcaggcccgggcaggcaagcggcggcctccgccatgaaccccaggggcctgttccaggacttcaaccccagtaagtttctcatctacacctgcctgctgctcttctcggtgctgctgcccctccgcctggacggcatcatccaatggagctactggggcgtctttgcccccatatggctgtggaagcttctagtcgtcgcaggcgcctccgtgggcgcgggcgtttgggcccgcaaccctcgctaccgcaccgagggagaggcctgtgtggagttcaaagccatgctgatcgctgtgggcatccacctgctgctgctcatgttcgaagtcctggtctgcgacagggtggagaggggcacccacttctggctgctggtcttcatgcctctcttcttcgtgtcccccgtgtccgtggctgcctgcgtctggggctttcgacacgataggtcgctggagctggagatcctgtgctcggtcaacatcctgcagttcatcttcatcgccctaaagctggacaggattattcactggccgtggctggtggtgtttgtgcccctgtggatcctcatgtcgttcctttgcctggtcgtcctctattacatcgtctggtccctcctgttcctgcggtccctggatgtggttgccgagcagcggagaacacacgtgaccatggctatcagttggataacgattgtcgtgcctctgctcacttttgaggtcctgctggttcacagattggatggccacaatacattctcctacgtctccatatttgtccccctttggctttccttactaactttaatggccacaacatttaggcgaaaggggggcaatcattggtggtttggcattcgcagagacttctgtcagtttctgcttgaaattttcccatttttaagagaatatgggaacatttcatatgatctccatcacgaagatagtgaagatgctgaagaaacatcagttccagaagctccgaaaattgctccaatatttggaaagaaggccagagtagttataacccagagccctgggaaatacgttcccccccctcccaagttaaatattgatatgccagattaaactcctagagaggacccaggcacacacagactccacctggccttcgcctcttgttcattcatcccaaacctggaaatggaaacaggcttcaaacactcgtctcacgccgtgtttgagatcaccgcctcatcagtatgcatcatagatggaggtggtttcagtatgtgggtgtgtgtggtgtgtacctgggtaagagacttgctttccaggttcgcactttcaggtgtagctgggggcagtaagtcgaattgttttagtaggtcctcaaaaggaataaccacacagctgtttgtttaaatgctactgtacctatcaaaactattgtttaaaaagtatttttatacactgctaatctaaaattgtatttcagattgtgcctgtcataacaatagcaaatgtaaaaagttctctttcccaccacttgtttataaacctcatagttgatatttttagtgttcctactgttaaaatactctctccttgggctttgctgatactggtctttaatattctgataggtgaatttttctaatggaatgaacccatgcatatatagtatttatatgaatattttagcagtgtaatatgttgaattctagttctctgcattaccattattacgttaaagtattttttaaagcttaaaaaaaaaaaaaaaaaaa

SEQ ID NO: 9 - protein, 350 aa, Mus sp.
MNLRGLFQDFNPSKFLIYACLLLFSVLLALRLDGIIQWSYWAVFAPIWLWKLMVIVGASVGTGVWARNPQYRAEGETCVEFKAMLIAVGIHLLLLMFEVLVCDRIERGSHFWLLVFMPLFFVSPVSVAACVWGFRHDRSLELEILCSVNILQFIFIALRLDKIIHWPWLVVCVPLWILMSFLCLVVLYYIVWSVLFLRSMDVIAEQRRTHITMALSWMTIVVPLLTFEILLVHKLDGHNAFSCIPIFVPLWLSLITLMATTFGQKGGNHWWFGIRKDFCQFLLEIFPFLREYGNISYDLHHEDSEETEETPVPEPPKIAPMFRKKARVVITQSPGKYVLPPPKLNIEMPD

SEQ ID NO: 10 - protein, 350 aa, Mus sp.
MNPRGLFQDFNPSKFLIYACLLLFSVLLPLRLDGIIQWSYWAVFAPIWLWKLLVIVGASVGAGVWARNPRYRTEGEACVEFKAMLIAVGIHLLLLMFEILVCDRVERGTHLWLLVFMPLFFVSPVSVAACVWGFRHDRSLELEILCSVNILQFIFIALRLDRIIHWPWLVVFVPLWILMSFLCLVVLYYIVWSLLFLRSLDVVAEQRRTHVTMAISWITIVVPLLIFEVLLVHRLDDHNTFSYISIFIPLWLSLLTLMATTFRRKGGNHWWFGIRRDFCQFLLEVFPFLREYGNISYDLHHEDSEDAEDASVSEAPKIAPMFGKKARVVITQSPGKYVPPPPKLNIDMPD

SEQ ID NO: 11 - protein, 350 aa, Homo sapiens
MNLRGLFQDFNPSKFLIYACLLLFSVLLALRLDGIIQWSYWAVFAPIWLWKLMVIVGASVGTGVWARNPQYRAEGETCVEFKAMLIAVGIHLLLLMFEVLVCDRIERGSHFWLLVFMPLFFVSPVSVAACVWGFRHDRSLELEILCSVNILQFIFIALRLDKIIHWPWLVVCVPLWILMSFLCLVVLYYIVWSVLFLRSMDVIAEQRRTHITMALSWMTIVVPLLTFEILLVHKLDGHNAFSCIPIFVPLWLSLITLMATTFGQKGGNHWWFGIRKDFCQFLLEIFPFLREYGNISYDLHHEDNEETEETPVPEPPKIAPMFRKKARVVITQSPGKYVLPPPKLNIEMPD

SEQ ID NO: 12 - protein, 166 aa, Homo sapiens
MNLRGLFQDFNPSKFLIYACLLLFSVLLALRLDGIIQWSYWAVFAPIWLWKLMVIVGASVGTGVWARNPQYRAEGETCVEFKAMLIAVGIHLLLLMFEVLVCDRIERGSHFWLLVFMPLFFVSPVSVAACVWGFRHDRSLEVRFHIFKNVFHFGRSRQVDHLRSGV

SEQ ID NO: 13 - protein, 72 aa, Homo sapiens
MNLRGLFQDFNPSKFLIYACLLLFSVLLALRLDGIIQWSYWAVFAPIWLWKLMVIVGASVGTGVWARNPQYR

SEQ ID NO: 14 - protein, 350 aa, Homo sapiens
MNPRGLFQDFNPSKFLIYTCLLLFSVLLPLRLDGIIQWSYWAVFAPIWLWKLLVVAGASVGAGVWARNPRYRTEGEACVEFKAMLIAVGIHLLLLMFEVLVCDRVERGTHFWLLVFMPLFFVSPVSVAACVWGFRHDRSLELEILCSVNILQFIFIALKLDRIIHWPWLVVFVPLWILMSFLCLVVLYYIVWSLLFLRSLDVVAEQRRTHVTMAISWITIVVPLLTFEVLLVHRLDGHNTFSYVSIFVPLWLSLLTLMATTFRRKGGNHWWFGIRRDFCQFLLEIFPFLREYGNISYDLHHEDSEDAEETSVPEAPKIAPIFGKKARVVITQSPGKYVPPPPKLNIDMPD

SEQ ID NO: 15 - DNA, 2570 bp, Homo sapiens
ttgggtaccgggccccccctcgagttttttttttttttttttttttgagacagagtctcactctgttgctcaggctacagtgcagtggcacgatcacagctcactgcatcctccaactactgggcccaagcaatcctcccacctcagccctgagtagctgggaccacaggtgtgtaccaccacacccaactattttttgctttttgtagagacggggtctccctgccctgtccaggctggtcttgaactcctgggctcaagcaatccttctgcctcagtctctcaaagtgctagggttacaggcacatatcaccgtgcccggccagatgccttctcttataattgatagaacaagtagacaaaatcagtaaggatataaaagggttgaacgttatcaatcaacttgacctaattagatatgtatagaacattccacccaatagcagactatgcattctcttcaagtgtacataacatttatcaagacagaccacattctgggccgtaagacaagtctcaataaatttaaagggattcagtttaaataaatcaaaaaggtcaggccacagtggaattaaattattaatcaataacagaaagatatctggaaaatccccaaatatttgcagagtaaataatacgcttctaagtatctcataggtcaaagaagtaaattagaaagtatttgaattgaacgaaaatgaaaacacaccatatcagaatttgtggaatgccactaaagcagtgcttagagggaaattcatagcagtaagcacctacattagaaaagaaagaaagagctgggcacggtggctcacgcctgtaattccaatacttttggaggccaaggcaggaggattgcttgagcccaggagctctaagaccagcctgggcaacacagagagacccccatccccgcaaataatttgaaacaattagctgggtgtggtggtatgcacctgtggtcccagccacctgagagaccggggcaggtacgtggatcccaggaagtcaaggatgcagtgagccatgattgtgccactgcactctaacatgggcaacagagtgaaaccttatctaccaaaaaaagaaaagaaaagaaaagaaaggttttgaatcaatgacatcagcttctaccttaagaaactagaaaaagaacaaatgagaactaccagggcagtgaagaacacacgtgaccatggccatcagctggataacgactgtcatcgtgccctgctcacctttgaagtcctgctggttcacagactggatggccgcaatacattctcctgtatctccatatctgtccccctttggcttttgttactaactttaatgaccacaacatttaggccaaaaaggggcaatcattggtggtttggtattcgcagagacttctgccagtttctgcttgaaattttcccatttttaagagaatatgggaacatttcatatgatctccatcaggaagatagcgaaggtgctgaagaaacattggttccagaagctccgaaaattgctccagtgtttgggaagaccagggtagttttaatccctgggaaatatgttcccccacctcccaagttaaatattgatatgccagattaaactcctagagagggcccatacacagactccaccttgcctttgcctcttgttcattcataccaaacctggaaatggaaacaggcaccaaacactgtcatctcataccctgtttgagattgatgcctcatcagcatgcatcataaatggagactgtttcagcatgtgggtgtgtgtggtgtgtacctgggtaagagaacttgctttccacattcgtgctttagatgtagctgggggcagtaagttgaactgttttagtaggtcctcaaaaggaataactacacagtggttttctttaaagtgctactgtacctatcgaaagtatcgtttaaaagtatttttatacactgctagtacaaaattgtatttcagattgtgcctgttatgacataatagcaaacgtaaagaattctctgtttataaacctcacagtttgtttaaactgcatattgtctattgcagatgtctttaaagattgcataaaacaatatgtgcctggtgtggagcttacccaagtactagacatgaacatagggactgcaaatcccatgggtgttaatatttaggtgttagtaaccgaggtctgcactgtgtgaaggtctctggtagtatccgttagtagagggagcagccaccgcccttgcgaacttgtgacatgctctagtgtagtaccaggccataaagtgacactgttgtttagcaccctgaatttttccacacaggtagtaactgtccagaaacaatcaaaataagcaacaagtggtttgtccatttctaagaatcttagaatattagttggttgtaatgtaaagcattacctgtcactggaaagctggagagagtggccttaaccagaagtggccaacaatcaggtattattttaaggtctaaacctcgtgccgaattcctgcagcccgggggatccactagtctagagcggccgccaccgcgggagctccagcttt

SEQ ID NO: 16 - protein, 148 aa, Homo sapiens
MRTTRAVKNTRDHGHQLDNDCHRALLTFEVLLVHRLDGRNTFSCISISVPLWLLLLTLMTTTFRPKRGNHWWFGIRRDFCQFLLEIFPFLREYGNISYDLHQEDSEGAEETLVPEAPKIAPVFGKTRVVLIPGKYVPPPPKLNIDMPD

SEQ ID NO: 17 - protein, 8 aa, Artificial Sequence
  Description of Artificial Sequence Synthetic peptide
DYKDDDDK

SEQ ID NO: 18 - protein, 22 aa, Homo sapiens
WWFGIRKDFCQFLLEIFPFLRE

SEQ ID NO: 19 - protein, 20 aa, Homo sapiens
VLVCDRIERGSHFWLLVFMP

SEQ ID NO: 20 - protein, 5 aa, Artificial Sequence
  Description of Artificial Sequence Synthetic peptide
GETCV

SEQ ID NO: 21 - protein, 11 aa, Artificial Sequence
  Description of Artificial Sequence Synthetic peptide
ELEILCSVNIL

SEQ ID NO: 22 - protein, 22 aa, Artificial Sequence
  Description of Artificial Sequence Synthetic peptide
LDGHNAFSCIPIFVPLWLSLIT

SEQ ID NO: 23 - DNA, 50 bp, Artificial Sequence
  Description of Artificial Sequence Synthetic probe
aacgaagggccagtagcacagagaacagcagcagacaggcatagatgagg

SEQ ID NO: 24 - DNA, 50 bp, Artificial Sequence
  Description of Artificial Sequence Synthetic probe
cctcatctatgcctgtctgctgctgttctctgtgctactggcccttcgtt

SEQ ID NO: 25 - protein, 18 aa, Artificial Sequence
  Description of Artificial Sequence Synthetic peptide
CLHHEDNEETEETPVPEP

SEQ ID NO: 26 - DNA, 20 bp, Artificial Sequence
  Description of Artificial Sequence Synthetic primer
ggtgtgggagaaatggctta

SEQ ID NO: 27 - DNA, 20 bp, Artificial Sequence
  Description of Artificial Sequence Synthetic primer
ataccagcagagcctggaga

SEQ ID NO: 28 - DNA, 25 bp, Artificial Sequence
  Description of Artificial Sequence Synthetic oligonucleotide
aattcctcatctatgcctgtctgct

SEQ ID NO: 29 - DNA, 40 bp, Artificial Sequence
  Description of Artificial Sequence Synthetic oligonucleotide
gctgttctctgtgctgctggcccttcgtttggatggcatc

SEQ ID NO: 30 - DNA, 40 bp, Artificial Sequence
  Description of Artificial Sequence Synthetic oligonucleotide
atgaacctgaggggcctcttccaggacttcaacccgagta

SEQ ID NO: 31 - DNA, 20 bp, Artificial Sequence
  Description of Artificial Sequence Synthetic oligonucleotide
tgctccaatatggctgtgga

SEQ ID NO: 32 - DNA, 20 bp, Artificial Sequence
  Description of Artificial Sequence Synthetic oligonucleotide
ctctagtgacctgtcatgtc

SEQ ID NO: 33 - DNA, 20 bp, Artificial Sequence
  Description of Artificial Sequence Synthetic oligonucleotide
gacagagcttaagtggactg

SEQ ID NO: 34 - DNA, 20 bp, Artificial Sequence
  Description of Artificial Sequence Synthetic oligonucleotide
tacagttcctactgactgcc

SEQ ID NO: 35 - DNA, 40 bp, Artificial Sequence
  Description of Artificial Sequence Synthetic oligonucleotide
acgcactctctccgccttcctctgccccctcgttcacccc

SEQ ID NO: 36 - DNA, 25 bp, Artificial Sequence
  Description of Artificial Sequence Synthetic oligonucleotide
gcagaccagaaccagtactggagct

SEQ ID NO: 37 - DNA, 40 bp, Artificial Sequence
  Description of Artificial Sequence Synthetic oligonucleotide
gggtctccaggtacgtccatctcatgccttgtttgcatcc

SEQ ID NO: 38 - DNA, 25 bp, Artificial Sequence
  Description of Artificial Sequence Synthetic oligonucleotide
attcctcatctatgcctgtctgctg

SEQ ID NO: 39 - DNA, 40 bp, Artificial Sequence
  Description of Artificial Sequence Synthetic oligonucleotide
ctgttctctgtgctactggcccttcgtttggatggcatca

SEQ ID NO: 40 - DNA, 40 bp, Artificial Sequence
  Description of Artificial Sequence Synthetic oligonucleotide
tgaacctgaggggcctctttcaggacttcaacccgagtaa

SEQ ID NO: 41 - DNA, 21 bp, Artificial Sequence
  Description of Artificial Sequence Synthetic oligonucleotide
ggatggcatcattcaatggag

SEQ ID NO: 42 - DNA, 21 bp, Artificial Sequence
  Description of Artificial Sequence Synthetic oligonucleotide
gaacaatggcatgaagaccag

SEQ ID NO: 43 - DNA, 21 bp, Artificial Sequence
  Description of Artificial Sequence Synthetic oligonucleotide
actgagctggatgaccattgt

SEQ ID NO: 44 - DNA, 21 bp, Artificial Sequence
  Description of Artificial Sequence Synthetic oligonucleotide
tcctcactatcttcatggtgg

SEQ ID NO: 45 - DNA, 25 bp, Artificial Sequence
  Description of Artificial Sequence Synthetic oligonucleotide
tcatcacccagagccctggcaagta

SEQ ID NO: 46 - DNA, 40 bp, Artificial Sequence
  Description of Artificial Sequence Synthetic oligonucleotide
cctaaaattgcacctatgttccgcaagaaggccagggtag

SEQ ID NO: 47 - DNA, 40 bp, Artificial Sequence
  Description of Artificial Sequence Synthetic oligonucleotide
tgtccttcctccacccaaactaaatattgaaatgccagac

SEQ ID NO: 48 - DNA, 20 bp, Artificial Sequence
  Description of Artificial Sequence Synthetic oligonucleotide
tgaactgcaggatgttgacc

SEQ ID NO: 49 - DNA, 20 bp, Artificial Sequence
  Description of Artificial Sequence Synthetic oligonucleotide
tcatccaatggagctactgg

SEQ ID NO: 50 - DNA, 40 bp, Artificial Sequence
  Description of Artificial Sequence Synthetic oligonucleotide
atgaaccccaggggcctgttccaggacttcaaccccagta

SEQ ID NO: 51 - DNA, 25 bp, Artificial Sequence
  Description of Artificial Sequence Synthetic oligonucleotide
agtttctcatctacacctgcctgct

SEQ ID NO: 52 - DNA, 40 bp, Artificial Sequence
  Description of Artificial Sequence Synthetic oligonucleotide
gctcttctcggtgctgctgcccctccgcctggacggcatc

SEQ ID NO: 53 - DNA, 40 bp, Artificial Sequence
  Description of Artificial Sequence Synthetic oligonucleotide
actcttctccgtgctgctgcccctgcgcctggacggcatc

SEQ ID NO: 54 - DNA, 25 bp, Artificial Sequence
  Description of Artificial Sequence Synthetic oligonucleotide
agttcctcatttatgcctgcttgct

SEQ ID NO: 55 - DNA, 20 bp, Artificial Sequence
  Description of Artificial Sequence Synthetic oligonucleotide
tggataatcctgtccagcct

SEQ ID NO: 56 - DNA, 20 bp, Artificial Sequence
  Description of Artificial Sequence Synthetic oligonucleotide
atcatccagtggagctactg

SEQ ID NO: 57 - DNA, 40 bp, Artificial Sequence; identical to SEQ ID NO: 50
  Description of Artificial Sequence Synthetic oligonucleotide

SEQ ID NO: 58 - DNA, 22 bp, Artificial Sequence
  Description of Artificial Sequence Synthetic oligonucleotide
tgtggaagctcctggtcatcgt

SEQ ID NO: 59 - DNA, 22 bp, Artificial Sequence
  Description of Artificial Sequence Synthetic oligonucleotide
gataatcctgtccagcctcagg

SEQ ID NO: 60 - DNA, 40 bp, Artificial Sequence
  Description of Artificial Sequence Synthetic oligonucleotide
gaagctcctggtcatcgtgggcgcctcggtgggtgcgggc

SEQ ID NO: 61 - DNA, 25 bp, Artificial Sequence
  Description of Artificial Sequence Synthetic oligonucleotide
gtgtgggcccgcaacccacgctacc

SEQ ID NO: 62 - DNA, 40 bp, Artificial Sequence
  Description of Artificial Sequence Synthetic oligonucleotide
gtacagagggggaagcctgcgtggaattcaaagccatgct

SEQ ID NO: 63 - DNA, 25 bp, Artificial Sequence
  Description of Artificial Sequence Synthetic oligonucleotide
acagagccctgggaaatatgtgcct

SEQ ID NO: 64 - DNA, 40 bp, Artificial Sequence
  Description of Artificial Sequence Synthetic oligonucleotide
ccacctcccaagttaaacattgatatgccagactaaactc

SEQ ID NO: 65 - DNA, 40 bp, Artificial Sequence
  Description of Artificial Sequence Synthetic oligonucleotide
ttgctccaatgtttggaaagaaggcgcgggtagttataac

SEQ ID NO: 66 - DNA, 40 bp, Artificial Sequence
  Description of Artificial Sequence Synthetic oligonucleotide
ccaccttgggcaccttggtgtctttcaaaagtgccaggct

SEQ ID NO: 67 - DNA, 25 bp, Artificial Sequence
  Description of Artificial Sequence Synthetic oligonucleotide
ccttcctgcctcagggcctttgcac

SEQ ID NO: 68 - DNA, 40 bp, Artificial Sequence
  Description of Artificial Sequence Synthetic oligonucleotide
ttgctgctccctccgtttgaaatactgtatcccagagagt

SEQ ID NO: 69 - DNA, 20 bp, Artificial Sequence
  Description of Artificial Sequence Synthetic oligonucleotide
ggcaccttggtgtctttcaa

SEQ ID NO: 70 - DNA, 20 bp, Artificial Sequence
  Description of Artificial Sequence Synthetic oligonucleotide
cagtctgaattaggagccag

SEQ ID NO: 71 - DNA, 20 bp, Artificial Sequence
  Description of Artificial Sequence Synthetic oligonucleotide
tcggagcttctggaaccaat

SEQ ID NO: 72 - DNA, 20 bp, Artificial Sequence
  Description of Artificial Sequence Synthetic oligonucleotide
ccatcagctggataacgact

SEQ ID NO: 73 - DNA, 40 bp, Artificial Sequence
  Description of Artificial Sequence Synthetic oligonucleotide
accatggccatcagctggataacgactgtcatcgtgcccc

SEQ ID NO: 74 - DNA, 25 bp, Artificial Sequence
  Description of Artificial Sequence Synthetic oligonucleotide
tgctcacctttgaagtcctgctggt

SEQ ID NO: 75 - DNA, 40 bp, Artificial Sequence
  Description of Artificial Sequence Synthetic oligonucleotide
tcacagactggatggccgcaatacattctcctgtatctcc

SEQ ID NO: 76 - DNA, 40 bp, Artificial Sequence
  Description of Artificial Sequence Synthetic oligonucleotide
aattttggtatatggtgcaaaaaaaggggtccaatttctt

SEQ ID NO: 77 - DNA, 25 bp, Artificial Sequence
  Description of Artificial Sequence Synthetic oligonucleotide
ctgcaactggccagccagttatctc

SEQ ID NO: 78 - DNA, 40 bp, Artificial Sequence
  Description of Artificial Sequence Synthetic oligonucleotide
agcatcattaattgaatagggaatccttaccccactgatt

SEQ ID NO: 79 - DNA, 20 bp, Artificial Sequence
  Description of Artificial Sequence Synthetic oligonucleotide
aactggccagccagttatct

SEQ ID NO: 80 - DNA, 20 bp, Artificial Sequence
  Description of Artificial Sequence Synthetic oligonucleotide
aatggattgttgggtgcagc

SEQ ID NO: 81 - DNA, 22 bp, Artificial Sequence
  Description of Artificial Sequence Synthetic oligonucleotide
ccagccagttatctcagcatca

SEQ ID NO: 82 - DNA, 22 bp, Artificial Sequence
  Description of Artificial Sequence Synthetic oligonucleotide
accatggcatgtgtatcccaga

SEQ ID NO: 83 - DNA, 55 bp, Artificial Sequence
  Description of Artificial Sequence Synthetic oligonucleotide
ggggacaagtttgtacaaaaaagcaggctaccatgaacctgaggggcctcttcca

SEQ ID NO: 84 - DNA, 64 bp, Artificial Sequence
  Description of Artificial Sequence Synthetic oligonucleotide
ggggaccactttgtacaagaaagctgggtcctaatctggcatttcgatatttaatttgggaggt

SEQ ID NO: 85 - DNA, 61 bp, Artificial Sequence
  Description of Artificial Sequence Synthetic oligonucleotide
ggggaccactttgtacaagaaagctgggtcgcatttcgatatttaatttgggaggtgggag

SEQ ID NO: 86 - DNA, 56 bp, Artificial Sequence
  Description of Artificial Sequence Synthetic oligonucleotide
ggggacaagtttgtacaaaaaagcaggctctaccatgaaccccaggggcctgttcc

SEQ ID NO: 87 - DNA, 64 bp, Artificial Sequence
  Description of Artificial Sequence Synthetic oligonucleotide
ggggaccactttgtacaagaaagctgggtcttaatctggcatatcaatatttaacttgggaggg

SEQ ID NO: 88 - DNA, 61 bp, Artificial Sequence
  Description of Artificial Sequence Synthetic oligonucleotide
ggggaccactttgtacaagaaagctgggtcatctggcatatcaatatttaacttgggaggg

SEQ ID NO: 89 - DNA, 56 bp, Artificial Sequence; identical to SEQ ID NO: 86
  Description of Artificial Sequence Synthetic oligonucleotide

SEQ ID NO: 90 - DNA, 62 bp, Artificial Sequence
  Description of Artificial Sequence Synthetic oligonucleotide
ggggaccactttgtacaagaaagctgggtcttagtctggcatatcaatgtttaacttgggag

SEQ ID NO: 91 - protein, 86 aa, Artificial Sequence
  Description of Artificial Sequence Synthetic peptide
KGGNHWWFGIRKDFCQFLLEIFPFLREYGNISYDLHHEDNEETEETPVPEPPKIAPMFRKKARVVITQSPGKYVLPPPKLNIEMPD

SEQ ID NO: 92 - DNA, 258 bp, Artificial Sequence
  Description of Artificial Sequence Synthetic nucleotide construct
aagggaggaaaccactggtggtttggtatccgcaaagatttctgtcagtttctgcttgaaatcttcccatttctacgagaatatggaaacatttcctatgatctccatcacgaagataatgaagaaaccgaagagaccccagttccggagccccctaaaatcgcacccatgtttcgaaagaaggccagggtggtcattacccagagccctgggaagtatgtgctcccacctcccaaattaaatatcgaaatgccagat

SEQ ID NO: 93 - DNA, 663 bp, Homo sapiens
  modified_base (57): a, c, g, t, unknown or other
  modified_base (73): a, c, g, t, unknown or other
  modified_base (124): a, c, g, t, unknown or other
  modified_base (136): a, c, g, t, unknown or other
  modified_base (138): a, c, g, t, unknown or other
  modified_base (140): a, c, g, t, unknown or other
  modified_base (282): a, c, g, t, unknown or other
  modified_base (310): a, c, g, t, unknown or other
  modified_base (620): a, c, g, t, unknown or other
  modified_base (649): a, c, g, t, unknown or other
ggggactcggccctgaacgagcaggagaaggagttgcagcggcggctgaagcgtctntacccggccgtggacnaacaagagacgccgttgcctcggtcctggagcccgaaggacaagttcagcntacatcggcctntntnagaacaacctgcgggtgcactacaaaggtcatggcaaaaccccaaaagatgccgcgtcagttcgagccacgcatccaataccagcagcctgtgggatttattattttgaagtaaaaattgtcagtaagggaagagatggttncatgggaattggtctttctgctcaaggngtgaacatgaatagactaccaggttgggataagcattcatatggttaccatggggatgatggacattcgttttgttcttctggaactggacaaccttatggaccaactttcactactggtgatgtcattggctgttgtgttaatcttatcaacaatacctgcttttacaccaagaatggacatagtttaggtattgctttcactgacctaccgccaaatttgtatcctactgtggggcttcaaacaccaggagaagtggtcgatgccaattttgggcaacatcctttcgtgtttgatatagaagactatntgcgggagtggagaaccaaaatccaggcncagatagatcgatt

SEQ ID NO: 94 - protein, 212 aa, Homo sapiens
  MOD_RES (104): variable amino acid
  MOD_RES (134): variable amino acid
  MOD_RES (190): variable amino acid
KEKVQGRVGRRAPGKAKPASPARRLDLRGKRSPTPGKGPADRASRAPPRPRSTTSQVTPAEEKDGHSPMSKGLVNGLKAGPMALSSKGSSGAPVYVDLAYIPNXCSGKTADLDFFRRVRASYYVVSGNDPANGXPSRAVLDALLEGKAQWGENLQVTLIPTHDTEVTREWYQQTHEQQQQLNVLVLASTXTVVMQDESFPACRLSSEKPPSL

SEQ ID NO: 95 - protein, 177 aa, Homo sapiens
KKESVEKAAKPTTTPEVKAARGEEKDKETKNAANASASKSAKTATAGPGTTKTTKSSAVPPGLPVYLDLCYIPNHSNSKNVDVEFFKRVRSSYYVVSGNDPAAEEPSRAVLDALLEGKAQWGSNMQVTLIPTHDSEVMREWYQETHEKQQDLNIMVLASSSTVVMQDESFPACKIEL

SEQ ID NO: 96 - protein, 105 aa, Artificial Sequence
  Description of Artificial Sequence Synthetic protein
  MOD_RES (86)..(97): variable amino acid
LLTFEILLVHKLDGHNAFSCIPIFVPLWLSLITLMATTFGQKGGNHWWFGIRKDFCQFLLEIFPFLRETYRGNISTYRDLHHEDNXXXXXXXXXXXXKIAPMFRK

SEQ ID NO: 97 - protein, 101 aa, Homo sapiens
LLTFEVLLVHRLDGRNTFSCISISVPLWLLLLTLMTTTFRPKRGNHWWFGIRRDFCQFLLEIFPFLREYGNISYDLHQEDSEGAEETLVPEAPKIAPVFGK

SEQ ID NO: 98 - protein, 17 aa, Artificial Sequence
  Description of Artificial Sequence Synthetic peptide
PGKYVLPPPKLNIEMPD

SEQ ID NO: 99 - protein, 17 aa, Homo sapiens
PGKYVPPPPKLNIDMPD

SEQ ID NO: 100 - protein, 18 aa, Artificial Sequence
  Description of Artificial Sequence Synthetic peptide
QRRTHITMALSWMTIVVP

SEQ ID NO: 101 - protein, 18 aa, Homo sapiens
QRTHVTMAISWITTVIVP

SEQ ID NO: 102 - protein, 350 aa, Mus musculus; identical to SEQ ID NO: 9

SEQ ID NO: 103 - protein, 148 aa, Homo sapiens; identical to SEQ ID NO: 16

SEQ ID NO: 104 - DNA, 1397 bp, Homo sapiens
aggtttagaccttaaaataatacctgattgttggccacttctggttaaggccactctctccagctttccagtgacaggtaatgctttacattacaaccaactaatattctaagattcttagaaatggacaaaccacttgttgcttattttgattgtttctggacagttactacctgtgtggaaaaattcagggtgctaaacaacagtgtcactttatggcctggtactacactagagcatgtcacaagttcgcaagggcggtggctgctccctctactaacggatactaccagagaccttcacacagtgcagacctcggttactaacacctaaatattaacacccatgggatttgcagtccctatgttcatgtctagtacttgggtaagctccacaccaggcacatattgttttatgcaatctttaaagacatctgcaatagacaatatgcagtttaaacaaactgtgaggtttataaacagagaattctttacgtttgctattatgtcataacaggcacaatctgaaatacaattttgtactagcagtgtataaaaatacttttaaacgatactttcgataggtacagtagcactttaaagaaaaccactgtgtagttattccttttgaggacctactaaaacagttcaacttactgcccccagctacatctaaagcacgaatgtggaaagcaagttctcttacccaggtacacaccacacacacccacatgctgaaacagtctccatttatgatgcatgctgatgaggcatcaatctcaaacagggtatgagatgacagtgtttggtgcctgtttccatttccaggtttggtatgaatgaacaagaggcaaaggcaaggtggagtctgtgtatgggccctctctaggagtttaatctggcatatcaatatttaacttgggaggtgggggaacatatttcccagggattaaaactacctggtcttcccaaacactggagcaattttcggagcttctggaaccaatgtttcttcagcaccttcgctatcttcctgatggagatcatatgaaatgttcccatattctcttaaaaatgggaaaatttcaagcagaaactggcagaagtctctgcgaataccaaaccaccaatgattgcccctttttggcctaaatgttgtggtcattaaagttagtaacaaaagccaaagggggacagatatggagatacaggagaatgtattgcggccatccagtctgtgaaccagcaggacttcaaaggtgagcagggcacgatgacagtcgttatccagctgatggccatggtcacgtgtgttcttcactgccctggtagttctcatttgttctttttctagtttcttaaggtagaagctgatgtcattgattcaaaacctttctt

SEQ ID NO: 105 - DNA, 1397 bp, Homo sapiens
aagtttagaccttaaaataatacctgattgttggccacttctggttaaggccactctctccagctttccagtgacaggtaatgctttacattacaaccaactaatattctaagattcttagaaatggacaaaccacttgttgcttattttgattgtttctggacagttactacctgtgtggaaaaattcagggtgctaaacaacagtgtcactttatggcctggtactacactagagcatgtcacaagttcgcaagggcggtggctgctccctctactaacggatactaccagagaccttcacacagtgcagacctcggttactaacacctaaatattaacacccatgggatttgcagtccctatgttcatgtctagtacttgggtaagctccacaccaggcacatattgttttatgcaatctttaaagacatctgcaatagacaatatgcagtttaaacaaactgtgaggtttataaacagagaattctttacgtttgctattatgtcataacaggcacaatctgaaatacaattttgtactagcagtgtataaaaatacttttaaacgatactttcgataggtacagtagcactttaaagaaaaccactgtgtagttattccttttgaggacctactaaaacagttcaacttactgcccccagctacatctaaagcacgaatgtggaaagcaagttctcttacccaggtacacaccacacacacccacatgctgaaacagtctccatttatgatgcatgctgatgaggcatcaatctcaaacagggtatgagatgacagtgtttggtgcctgtttccatttccaggtttggtatgaatgaacaagaggcaaaggcaaggtggagtctgtgtatgggccctctctaggagtttaatctggcatatcaatatttaacttgggaggtgggggaacatatttcccagggattaaaactacctggtcttcccaaacactggagcaattttcggagcttctggaaccaatgtttcttcagcaccttcgctatcttcctgatggagatcatatgaaatgttcccatattctcttaaaaatgggaaaatttcaagcagaaactggcagaagtctctgcgaataccaaaccaccaatgattgcccctttttggcctaaatgttgtggtcattaaagttagtaacaaaagccaaagggggacagatatggagatacaggagaatgtattgcggccatccagtctgtgaaccagcaggacttcaaaggtgagcagggcacgatgacagtcgttatccagctgatggccatggtcacgtgtgttcttcactgccctggtagttctcatttgttctttttctagtttcttaaggtagaagctgatgtcattgattcaaaacctttctt

SEQ ID NO: 106 - DNA, 367 bp, Homo sapiens
aattttggtatatggtgcaaaaaaaggggtccaatttcttctgcaactggccagccagttatctcagcatcattaattgaatagggaatccttaccccactgattgtttttgtcaggtttgtcaaagatgaaatagttgtaggtgtatggtcttatttctgggttccccattctgttccactggtatatgtgtctggttttgaactagtgccatgctgttttggttactatagccctgtttaaaatcaaatggagtgatgccgccacgttgtatttattttatttttttattatactttaagttctgggatacacatgccatggtggtttgctgcacccaacaatccattatctatgttgttttctc

SEQ ID NO: 107 - DNA, 133 bp, Homo sapiens
ccaccttgggcaccttggtgtctttcaaaagtgccaggctccttcctgcctcagggcctttgcacttgctgctccctccgtttgaaatactgtatcccagagagtcccatttctggctcctaattcagactga

SEQ ID NO: 108 - protein, 29 aa, Artificial Sequence
  Description of Artificial Sequence Synthetic peptide
RLDKIIHWPWLVVCVPLWILMSFLCLVVL

SEQ ID NO: 109 - protein, 30 aa, Homo sapiens
RIMSSLNWDSLTSCLPIWMTFISFSCLIAL

SEQ ID NO: 110 - protein, 47 aa, Artificial Sequence
  Description of Artificial Sequence Synthetic peptide
VGIHLLLLMFEVLVCDRIERGSHFWLLVFMPLFFVSPVSVAACVWGF

SEQ ID NO: 111 - protein, 49 aa, Homo sapiens
IGCCFLIVCFVCFVEDQMYVGLQHYFWALYSVPLFCVSVFVPVPCSFSY

SEQ ID NO: 112 - DNA, 88 bp, Homo sapiens
atcatgtcatctctaaactgggatagtttgacttcctgtcttcctatttggatgacttttatttctttctcttgcctgattgctctgg

SEQ ID NO: 113 - DNA, 148 bp, Homo sapiens
atagggtgttgtttcctcattgtttgttttgtctgctttgtggaagatcagatgtatgtaggtttgcagcattatttctgggctctctattctgttcctttgttctgtgtgtctgtgtttgtaccagtaccatgttcttttagttact

SEQ ID NO: 114 - protein, 19 aa, Homo sapiens
FLIYACLLLFSVLLALRLD

SEQ ID NO: 115 - protein, 17 aa, Homo sapiens
YWAVFAPIWLWKLMVIV

SEQ ID NO: 116 - protein, 20 aa, Homo sapiens
AMLIAVGIHLLLLMFEVLVC

SEQ ID NO: 117 - protein, 23 aa, Homo sapiens
WLLVFMPLFFVSPVSVAACVWGF

SEQ ID NO: 118 - protein, 24 aa, Homo sapiens
WLVVCVPLWILMSFLCLVVLYYIV

SEQ ID NO: 119 - protein, 17 aa, Homo sapiens
ITMALSWMTIVVPLLTF

SEQ ID NO: 120 - protein, 19 aa, Homo sapiens
AFSCIPIFVPLWLSLITLM

SEQ ID NO: 121 - DNA, 2267 bp, Xenopus laevis
cccgggcacgttaccgtattgatgttactagtagcgcacagaaacatcctggtctaagcagttgcagcaggtactgcgttgtagtggcggtagttacgactctgtaggttagagcggaggctttgctggagcaatgtccgcctagtgaagctcggagaggtgctcgcaccatgaatcttaggggcctcttccaggattttaaccccagtaaatttctcatctacgcatgtttgttgctcttttctgttctcctttccctgcgactggataatattattcagtggagttactgggcggtgtttgctccaatatggttgtggaaactaatggttattgtgggagcctcagttggtacaggtgtatgggcacgtaaccctcaatacagggcagaaggtgaaacatgtgtggagttcaaggccatgctaattgcagtgggaattcatttgctgcttcttatgtttgaagttcttgtttgcgatcgtattgaaagaggaaaccactacttctggttgctagtctttatgcctttattctttgtgtccccagtatccgttgcagcttgcgtttggggctttcggcatgatcgatcattggaattggaaatcttgtgctccgtcaatattctgcagtttatattcattgccctaagacttgacagcatcatcacttggccttggcttgtggtatgtgtcccgctgtggatccttatgtccttcctgtgcctagtagttctgtattatattgtgtggtcagttctgttcctgcgttcaatggatgttattgcagaacaaaggagaactcatattactatggcagtcagttggatggctatagttgtaccgcttctgacattcgagatattacttgttcatcgacttgatgggcacaatccattatcgaatatccctatatttgttccgctttggctttccttaataacgttgatggcaacaacctttggacagaaaggaggcaatcactggtggtttgggattcgtaaagacttctgccagtttctgttggagattttcccttttcttcgagaatatggcaatatctcatatgatattcatcatgaagacagtgaagatgctgaagaaacacctgtaccggagccccccaaaatcgcaccaatgtttcgaaagaagactggcgttgtcattacccagagcccagggaaatatattgttcctcctgctaaacttaacatcgacatgccggattaaggtgaaatttggtggcttgagggcacttttttctgttttaactaatcctgttagtagtacactatcaggtgtcatggactgaagggaaaaaaagactactgacctcattccttttttgtattcatttgtaattttttttgttcctgcaatggtatgtgttttcccattcctaattcatgtcatcatgttactcaagatcagggaagcttcttaagggcaaagaatgctggaatttgtagtttataatttgtggatgactataaattttcacatctgttgtcttggtaatgactgcagtcttgcattctagtttctagtaacacagagatagaccagctgtggccctccagatactgagctaacaagctttgggagacatcctgggaatcttagcagctctggggccacaggttggacttctcagcagtaaaattaagtataatgtttatcttaagtaaatgtctttgtgtgtgttgttatgcaatgcagctattgtttgatatctttacagcagaacttgtgcatagaattgaattcaagttgtgagctgttttataccactataaaaatacttttaaaaaaaaatctgtttaagggtcaagcattaccttggagaagtgatatttgagcagagggcttatgggatatatctaatatacaccttcccttaggagttactactccttgctcacttgtatagtatttataagaacattttatcaatgtaatatattgtgttcaaaattattcttatgtacagtataaatggataaatacaaagtatttttttaaataaaagatgtaaaatacatataagttgtcaaaattttgtttgtaatttacattttaaaatgatctatgtgaattctacaatgaaaaaagatctatacaatttcaaaagccagtatgtcatttttatatactgaccatgtacatattatgtaagatgtaaagccaaacaccaatgacatgaatgttaagttattagactatgaataaaacattgattttattttatgttgtaaaaaaaaaaaaaaaaaa

SEQ ID NO: 122 - DNA, 1056 bp, Xenopus laevis
atgaatcttaggggcctcttccaggattttaaccccagtaaatttctcatctacgcatgtttgttgctcttttctgttctcctttccctgcgactggataatattattcagtggagttactgggcggtgtttgctccaatatggttgtggaaactaatggttattgtgggagcctcagttggtacaggtgtatgggcacgtaaccctcaatacagggcagaaggtgaaacatgtgtggagttcaaggccatgctaattgcagtgggaattcatttgctgcttcttatgtttgaagttcttgtttgcgatcgtattgaaagaggaaaccactacttctggttgctagtctttatgcctttattctttgtgtccccagtatccgttgcagcttgcgtttggggctttcggcatgatcgatcattggaattggaaatcttgtgctccgtcaatattctgcagtttatattcattgccctaagacttgacagcatcatcacttggccttggcttgtggtatgtgtcccgctgtggatccttatgtccttcctgtgcctagtagttctgtattatattgtgtggtcagttctgttcctgcgttcaatggatgttattgcagaacaaaggagaactcatattactatggcagtcagttggatggctatagttgtaccgcttctgacattcgagatattacttgttcatcgacttgatgggcacaatccattatcgaatatccctatatttgttccgctttggctttccttaataacgttgatggcaacaacctttggacagaaaggaggcaatcactggtggtttgggattcgtaaagacttctgccagtttctgttggagattttcccttttcttcgagaatatggcaatatctcatatgatattcatcatgaagacagtgaagatgctgaagaaacacctgtaccggagccccccaaaatcgcaccaatgtttcgaaagaagactggcgttgtcattacccagagcccagggaaatatattgttcctcctgctaaacttaacatcgacatgccggattaa

SEQ ID NO: 123 - protein, 351 aa, Xenopus laevis
MNLRGLFQDFNPSKFLIYACLLLFSVLLSLRLDNIIQWSYWAVFAPIWLWKLMVIVGASVGTGVWARNPQYRAEGETCVEFKAMLIAVGIHLLLLMFEVLVCDRIERGNHYFWLLVFMPLFFVSPVSVAACVWGFRHDRSLELEILCSVNILQFIFIALRLDSIITWPWLVVCVPLWILMSFLCLVVLYYIVWSVLFLRSMDVIAEQRRTHITMAVSWMAIVVPLLTFEILLVHRLDGHNPLSNIPIFVPLWLSLITLMATTFGQKGGNHWWFGIRKDFCQFLLEIFPFLREYGNISYDIHHEDSEDAEETPVPEPPKIAPMFRKKTGVVITQSPGKYIVPPAKLNIDMPD

SEQ ID NO: 124 - DNA, 1292 bp, Danio rerio
ctcgagcactgttggcctactgggatgtgagtgccagtcagctagccagcctctccttttcagttcatgtaactatggtctgaagaggaaaccatgaatctccgaggcgttttccaagatttcaaccccagtaagttcctgatctacgcatgtctgctgctcttctctgtgctgctgtcactgaggctggatggcatcatccagtggagctactgggccgtgtttgcgcccatctggctctggaagctcatggtcatcatcggggcgtctgtgggcactggagtgtgggctcacaacccgcagtacagggctgaaggggagacgtgtgtggagtttaaggccatgctgatcgcagtgggaatccacctgctcctgctcaccttcgaggtgctggtctgcgagcgcgtggaacgggcttcgatcccctactggctcctggtgttcatgccgctcttcttcgtctctccggtgtcagtggcagcgtgtgtgtggggattcagacacgaccgctcgctggagctggagattctgtgctctgtaaatattcttcagtttatcttcatcgctctgaaactggacgggatcatcagctggccgtggctggtggtgtgtgtgccgctctggatcctcatgtccttcttgtgtctggtggtcctctattatatcgtgtggtctgtgctgtttctgcgctccatggatgtgatcgcggagcagcggcgcacacacatcaccatggccatcagctggatgactatagtcgtgcccctgctcacttttgagattctcctcgtccacaagctggataatcattatagccccaactacgtcccggtgtttgttcctctctgggtttctttagtgactctaatggtgaccacatttggccagaaaggaggcaatcactggtggtttggcatccgtaaagacttctgccagtttctgctggagctcttcccgttcctcagggaatatggcaacatctactatgacctgcatcacgaggactcagacatgtccgaggagttgcccattcacgaggtgcccaaaatccctaccatgtttagcaagaagacgggggtggtgatcacccaaagccctgggaaatactttgtgcccccacccaaactgtgcatcgacatgccagactaacattggagctctcgtatacagtatagcactatgcaatggaattcgctttgttacgtgctgttgaagacggcaacaacaatcccattaaactcggctcttgtttcctaaaaaaaatagctgcgcaaacggacctgttgacatca

SEQ ID NO: 125 - DNA, 1056 bp, Danio rerio
atgaatctccgaggcgttttccaagatttcaaccccagtaagttcctgatctacgcatgtctgctgctcttctctgtgctgctgtcactgaggctggatggcatcatccagtggagctactgggccgtgtttgcgcccatctggctctggaagctcatggtcatcatcggggcgtctgtgggcactggagtgtgggctcacaacccgcagtacagggctgaaggggagacgtgtgtggagtttaaggccatgctgatcgcagtgggaatccacctgctcctgctcaccttcgaggtgctggtctgcgagcgcgtggaacgggcttcgatcccctactggctcctggtgttcatgccgctcttcttcgtctctccggtgtcagtggcagcgtgtgtgtggggattcagacacgaccgctcgctggagctggagattctgtgctctgtaaatattcttcagtttatcttcatcgctctgaaactggacgggatcatcagctggccgtggctggtggtgtgtgtgccgctctggatcctcatgtccttcttgtgtctggtggtcctctattatatcgtgtggtctgtgctgtttctgcgctccatggatgtgatcgcggagcagcggcgcacacacatcaccatggccatcagctggatgactatagtcgtgcccctgctcacttttgagattctcctcgtccacaagctggataatcattatagccccaactacgtcccggtgtttgttcctctctgggtttctttagtgactctaatggtgaccacatttggccagaaaggaggcaatcactggtggtttggcatccgtaaagacttctgccagtttctgctggagctcttcccgttcctcagggaatatggcaacatctactatgacctgcatcacgaggactcagacatgtccgaggagttgcccattcacgaggtgcccaaaatccctaccatgtttagcaagaagacgggggtggtgatcacccaaagccctgggaaatactttgtgcccccacccaaactgtgcatcgacatgccagactaa

SEQ ID NO: 126 - protein, 351 aa, Danio rerio
MNLRGVFQDFNPSKFLIYACLLLFSVLLSLRLDGIIQWSYWAVFAPIWLWKLMVIIGASVGTGVWAHNPQYRAEGETCVEFKAMLIAVGIHLLLLTFEVLVCERVERASIPYWLLVFMPLFFVSPVSVAACVWGFRHDRSLELEILCSVNILQFIFIALKLDGIISWPWLVVCVPLWILMSFLCLVVLYYIVWSVLFLRSMDVIAEQRRTHITMAISWMTIVVPLLTFEILLVHKLDNHYSPNYVPVFVPLWVSLVTLMVTTFGQKGGNHWWFGIRKDFCQFLLELFPFLREYGNIYYDLHHEDSDMSEELPIHEVPKIPTMFSKKTGVVITQSPGKYFVPPPKLCIDMPD

SEQ ID NO: 127 - DNA, 20 bp, Artificial Sequence
  Description of Artificial Sequence Synthetic primer
accccaccgtgttcttcgac

SEQ ID NO: 128 - DNA, 20 bp, Artificial Sequence
  Description of Artificial Sequence Synthetic primer
catttgccatggacaagatg

SEQ ID NO: 129 - protein, 17 aa, Homo sapiens
LHHEDNEETEETPVPEP

SEQ ID NO: 130 - DNA, 20 bp, Artificial Sequence
  Description of Artificial Sequence Synthetic primer
ggtgtgggagaaatggctta

SEQ ID NO: 131 - DNA, 20 bp, Artificial Sequence
  Description of Artificial Sequence Synthetic primer
ataccagcagagcctggaga

SEQ ID NO: 132 - protein, 350 aa, Homo sapiens; identical to SEQ ID NO: 11

SEQ ID NO: 133 - protein, 350 aa, Homo sapiens; identical to SEQ ID NO: 14

SEQ ID NO: 134 - protein, 141 aa, Homo sapiens
RTHVTMAISWITTVIVPLLTFEVLLVHRLDGRNTFSCISISVPLWLLLLTLMTTTFRPKRGNHWWFGIRRDFCQFLLEIFPFLREYGNISYDLHQEDSEGAEETLVPEAPKIAPVFGKTRVVLIPGKYVPPPPKLNIDMPD

SEQ ID NO: 135 - protein, 358 aa, Drosophila sp.
MNLESLFRDFNPCKFIVHCSLFIFVTLFALRLDNYIDWPYWAIFTPLWIWKCTAILGAIVGAIVWCRYPHYRLEGDAYTQFKAMLISLALHLILLMFELLACDKLTSDRHLWVLVFIPLIFGSVVSVGACVWAVKHDRSFELELFLAVNALQFVSLPLKLDRFVYWNWDVVFVPMWIVICLSLVSVLYNIIFCGIMMRTPEVSLQQKKAALNAAVGNICTVLPLLCFQVVICDKLDGELKFPYIVVFSPLLVSILALIVLSSSAKGGNMWWFGIRKSFSQFLLSAMPFLQEYGNISYHPETHSNAAQSMPLDAASSSTSMAASEQLHEFHKHDKKSKKGAKNDILKPVVPFVSIDLPD

SEQ ID NO: 136 - protein, 432 aa, Caenorhabditis elegans
MVELSLGVFFRSFNASKVILCACLLIFTALFTLKLDGKVSFSYAFVFAPLWACNLLVFVGAIVGICSFCSKPPSRNEIVMRVDFSAMLITATEHIFLTFVSLVFIKLEFDYLFEPGYPLPWTIVFCPLFSLSILSIGIAIWSLRHDKPFEFEFFYAINIVQLVFIAFKLDKQVDWTWAVVFIPLWVVLSLAAVGVLYALVLSVVLIRSRHFIPAHRRQHVYSAILHTFFVIPALVCLVLLTGKLDSMNWPDKGSVSELSYTLAMAPLQVSFFFMAIMSAGFGGPSSTPQTNIWWFGLRKPLCPFLLEKCPSLRTYANPENGEEREAVIGEENEERDEADVGAIVNGNERRRNQRDKGRGSGGSQNSQEGGQNLYEKKYFFYIKNHKNSKFSPKKNYNPSEQIASSSAAIIRAMAQYRNEFGYQWKGDISQPD

SEQ ID NO: 137 - protein, 133 aa, Artificial Sequence
  Description of Artificial Sequence Synthetic protein
MRMSLAQRVLLTWLFTLLFLIMLVLKLDEKAPWNWFLIFIPVWIFDTILLVLLIVKMAGRCKSGFDPRHGSHNIKKKAWYLIAMLLKLAFCLALCAKLEQFTTMNLSYVFIPLWALLAGALTELGYNVFFVRD

SEQ ID NO: 138 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 139 - protein, 350 aa, Homo sapiens
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV

SEQ ID NO: 140 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 141 - protein, 5 aa, Artificial Sequence
  Description of Artificial Sequence Synthetic peptide
  MOD_RES (3): variable amino acid
GNXLV

SEQ ID NO: 142 - protein, 5 aa, Artificial Sequence
  Description of Artificial Sequence Synthetic peptide
  MOD_RES (3): variable amino acid
NPXIY

SEQ ID NO: 143 - protein, 10 aa, Artificial Sequence
  Description of Artificial Sequence Synthetic peptide
GNLLVILVIL

SEQ ID NO: 144 - protein, 5 aa, Artificial Sequence
  Description of Artificial Sequence Synthetic peptide
  MOD_RES (2)..(4): variable amino acid
GXXXS

SEQ ID NO: 145 - protein, 10 aa, Artificial Sequence
  Description of Artificial Sequence Synthetic peptide
  MOD_RES (3)..(4): variable amino acid
  MOD_RES (6)..(9): variable amino acid
QGXXVXXXXC

SEQ ID NO: 146 - protein, 273 aa, Artificial Sequence
  Description of Artificial Sequence Synthetic protein
ALLLKVIYTVGYSLSSLVCLLLAIAIFLLFRKLRCTRNYIHMNLFLSFILRALSFLIGDAVLLNSGCKVVAVFLHYFFLANFFWMLVEGLYLYTLLVVTVEVFFSERKRLWWYLLIGWGVPAVFVTIWAIVRPDKYGPILAEGPAGYGNEGCCWLSNDTNSGFWWIIKGPILLIILVNFIFFINILRILVQKLRIDSLSPQTGETDQYRKKRLVKSTLLLLPLLGVTWILFLFAPEDQSQGTLSLVFLYLFLILNSFQGFFVAVLYCFLNGEV

SEQ ID NO: 147 - protein, 283 aa, Artificial Sequence
  Description of Artificial Sequence Synthetic protein
LGIVLVALAVLGLIVLTLFVLVVFVKHRDTPIVKASNRELSYLLLIGLILCYLCSFLFIGKGTVEKPSTWSCILRRILFGLGFTLCYSALLAKTIRVLRIFRGAKKPGSTQKPKFISPWAQVLIVLILTLIQVIICVIWLVVEPPRPDIDIYSEKEKEEDEDVSIAPIILECNKGSVVAFLVVLGYDGLLAVLCTLLAFLTRNLPVDNFNEAKFITFSMYNVLTFCIVWVAFIPAYLSTPGSAEDYKVQVAVEIFSILASSTVLLGCLFAPKCYIILFKPEKN

SEQ ID NO: 148 - protein, 10 aa, Artificial Sequence
  Description of Artificial Sequence Synthetic peptide
RTKKLRTPTN

SEQ ID NO: 149 - protein, 26 aa, Artificial Sequence
  Description of Artificial Sequence Synthetic peptide
IFILNLAVADLLFLLTLPPWALYYLV

SEQ ID NO: 150 - protein, 211 aa, Artificial Sequence
  Description of Artificial Sequence Synthetic protein
GGSEDWPFGSALCKLVTALDVVNMYASILLLTAISIDRYLAIVHPLRYRRRRTSPRRAKVVILLVWVLALLLSLPPLLFSWVKTVEEGNGTLNVNVTVCLIDFPEESTASVSTWLRSYVLSTLVGFLLPLLVILVCYTRILRTLRKAAKTLLVVVVVFVLCWLPYFIVLLLDTLCLIIMSSTCELERVLPTALLVTLWLAYVNSCLNPIIY

(19) United States
(12) Patent Application Publication
(10) Pub. No.: US 2007/0157326 A1
(43) Pub. Date: Jul. 5, 2007

(54) Ee3-protein family and corresponding dna sequence

(75) Inventors: Armin Schneider, Heidelberg (DE); Martin Maurer, Heidelberg (DE); Wolfgang Kuschinsky, Heidelberg (DE); Sylvia Grunewald, Heildelberg (DE); Nikolaus Gassler, Heidelberg (DE)

Correspondence Address: FOLEY AND LARDNER LLP;SUITE 500, 3000 K STREET NW, WASHINGTON, DC, 20007, US

(73) Assignee: AXARON BIOSCIENCE AG

(21) Appl. No.: 10/492,911

(22) PCT Filed: Oct. 18, 2002
(86) PCT No.: PCT/EP02/11698

§ 371 Date: Oct. 20, 2004

(30) Foreign Application Priority Data
Oct. 18, 2001 (DE) 101 51 511.1

(51) Int. Cl. A01K 67/027; C12Q 1/68; C07H 21/04; C12P 21/06; C12N 5/08; C07K 14/705; C07K 16/28

(52) U.S. Cl. 800/14; 435/6; 435/69.1; 435/320.1; 435/325; 530/350; 536/23.5; 435/366; 530/388.22; 514/44

(57) ABSTRACT

The present invention relates to (i) DNA sequences, (ii) expression vectors comprising DNA sequences of the invention, (iv) host cells having the expression vectors of the invention, (v) gene products encoded by sequences of the invention, (vi) transgenic animals altered with respect to sequences of the invention, (vii) antibodies directed against gene products of the invention, (viii) methods for expressing and/or isolating gene products of the invention, (ix) the use of DNA sequences and/or gene products of the invention as drugs, (x) pharmaceutically active compounds and methods for preparing them and also uses of such compounds of the invention and (xi) nontherapeutic uses of DNA sequences and/or gene products of the invention.

The present invention relates to (i) DNA sequences, (ii) expression vectors comprising DNA sequences of the invention, (iv) host cells having the expression vectors of the invention, (v) gene products encoded by sequences of the invention, (vi) transgenic animals altered with respect to sequences of the invention, (vii) antibodies directed against gene products of the invention, (viii) methods for expressing and/or isolating gene products of the invention, (ix) the use of DNA sequences and/or gene products of the invention as drugs, (x) pharmaceutically active compounds and methods for preparing them and also uses of such compounds of the invention and (xi) nontherapeutic uses of DNA sequences and/or gene products of the invention.

Numerous proteins belonging to the class of G protein-coupled receptors (GPCRs) are known from the prior art. They constitute the largest family of surface molecules involved in signal transduction. They are activated by a large variety of ligands and other stimuli, for example light (rhodopsin), smells, (odorant receptors), calcium, amino acids or biogenic amines, nucleotides, peptides, fatty acids and fatty acid derivatives, and various polypeptides. It is assumed that approx. 1500 different proteins of the class of GPCRs exist in mammals, with approx. 1200 coding for olfactory, taste or vomeronasal receptors. The total number of “orphan” GPCRs (i.e. receptors which have been unable to be associated with any functionality up until now) is estimated to be 200-500 (Howard A D, McAllister G, Feighner S D, Liu Q, Nargund R P, Van der Ploeg L H, Patchett A A (2001) Orphan G protein-coupled receptors and natural ligand discovery. Trends Pharmacol Sci 22:132-140.). In C. elegans, GPCR sequences make up approx. 5% of the genome and code for approx. 1000 GPCR proteins (Bargmann C I (1998) Neurobiology of the Caenorhabditis elegans genome. Science 282:2028-2033 and Bargmann C I, Kaplan J M (1998) Signal transduction in the Caenorhabditis elegans nervous system. Annu Rev Neurosci 21:279-308).

It was found in the prior art that Drosophila melanogaster has approx. 200 GPCR sequences (Brody T, Cravchik A (2000) Drosophila melanogaster G protein-coupled receptors. J Cell Biol 150:83-88). This large group of topologically similar molecules is believed to have developed in a convergent manner, with the aim of coupling to G proteins.

According to the prior art, the class of GPCRs is divided into 3 or 4 families. Family A has by far the most members and includes, for example, also the odorant receptors (Buck L, Axel R (1991) A novel multigene family may encode odorant receptors: a molecular basis for odor recognition. Cell 65:175-187.). Family B includes receptors for secretin, VIP, and calcitonin. Family C comprises receptors such as the metabotropic glutamate receptors, the calcium receptors, the GABA-B receptors, the taste receptors, and the pheromone receptors. Virtually all of the “orphan” GPCR sequences, however, belong to family A.

One characteristic of the GPCR families is their signal transduction via G proteins. Binding of an extracellular ligand induces activation of a G protein which then transduces the signal. There are approx. 200 different G proteins, and each type of cell may have a different set. The active form of a G protein is the GTP-bound one, with said G protein being bound to GDP in the inactive state. Since the G protein is a GTPase, it inactivates itself after GTP binding. For this reason, signal transduction via G proteins is always a transient event. Each G protein consists of 3 subunits, alpha, beta and gamma. The alpha subunit is capable of binding GTP and is therefore able to control substantially the downstream messengers (“second messenger” systems). G protein Gs, for example, activates (stimulates) adenylate cyclase and thus leads to an increase in concentration of the intracellular messenger cAMP. G protein Gi inhibits adenylate cyclase, and Gq activates phospholipase C (second messengers: inositol triphosphate and diacylglycerol). Other examples of frequently utilized second messenger systems are calcium, K, cGMP and others. There are also chimeric G proteins. G-beta and -gamma subunits may likewise cause signal transduction, after they have decoupled from the trimeric protein complex, for example possible in the activation of MAP kinase signal pathways. The specificity of G protein coupling of a particular GPCR is an important pharmacological characteristic which may be utilized, inter alia, for developing assays, typically with determination of changes in the concentrations of the downstream messengers, for example calcium, cAMP or inositol triphosphate. Very recently, preliminary results in the literature have also drawn attention to the MAP kinase signal pathways which can likewise transduce GPCR signals (Marinissen M J, Gutkind J S (2001) G protein-coupled receptors and signaling networks: emerging paradigms. Trends Pharmacol Sci 22:368-376.).

Finally, a G protein-independent signal transduction is also possible in principle: thus, for example, direct interaction of the beta-2-adrenergic receptor with the NHERF protein modulates the activity of an Na/H exchanger (Hall R A, Premont R T, Chow C W, Blitzer J T, Pitcher J A, Claing A, Stoffel R H, Barak L S, Shenolikar S, Weinman E J, Grinstein S, Lefkowitz R J (1998) The beta2-adrenergic receptor interacts with the Na+/H+-exchanger regulatory factor to control Na+/H+ exchange. Nature 392:626-630).

Another characteristic which applies to many, if not all, GPCR receptors are oligomerizations. Particularly interesting here are heterodimerizations between different GPCRs, which may alter the pharmacological profile and ligand specificity (Bouvier M (2001) Oligomerization of G protein-coupled transmitter receptors. Nat Rev Neurosci 2:274-286). Thus, for example, the GABA-B receptor only functions as a heterodimer between GBR1 and GBR2 (Kuner R, Kohr G, Grunewald S, Eisenhardt G, Bach A, Kornau H C (1999) Role of heteromer formation in GABAB receptor function. Science 283:74-77). Since then, heterodimerization of this kind has been described for quite a number of GPCRs, for example mGluR5, the delta-opioid receptor, and others. Very recently, evidence was found, in the case of preeclampsia, for increased expression of one partner in a GPCR heterodimer pair causing a disorder. Here, increased expression of the bradykinin II receptor results in an increased formation of bradykinin II-angiotensin II receptor heterodimers whose altered pharmacological response may explain the hypertonic phenotype (AbdAlla, Lother, Massiery und Quitterer, Nat. Medicine (2001), 7, 1003-1009).

Finally, the proteins of the GPCR class are preferred pharmacological target molecules. More than 25% of the 100 best-selling medicaments pharmacologically target the GPCR-class proteins (Flower et al., 1999, Biochim. Biophys. Acta, 1422, 207-234). Thus, agonists and antagonists of the following receptor groups, in particular, are of the greatest pharmacological importance: the group of adreno receptors, the angiotensin II receptor, serotonin receptors, dopamine receptors, histamine receptors, leukotriene/prostaglandin receptors. Pharmaceuticals acting on said receptors cover a therapeutically broad spectrum of diseases, ranging from psychiatric symptoms (schizophrenias, depressions), via influencing hypertension to emergency medicaments for cardiac arrest. Known examples of customary medicaments acting on said receptors are, for example, alpha-adrenoceptor agonists (norfenefrine), beta-adrenoceptor agonists (isoprenaline, fenoterol), alpha-adrenoceptor blockers (prazosin), beta-adrenoceptor blockers (propanolol), 5-HT antagonists (cyproheptadine), H2 receptor blockers (cimetidine), H1 receptor blockers (terfenadine) dopamine agonists (bromocriptine), and others.

Despite intensive research efforts, however, the signal transduction pathways influenced by said receptors have still insufficiently been elucidated. In addition, there is a lack of a deeper understanding of the complex network of mutual influencing of the various GPCR systems and their action on downstream intracellular processes, in particular also with regard to external physiological states.

It is an object of the present invention to find further members of the class of GPCR proteins and the nucleotide sequences on which the latter are based. Another object of the present invention is to provide, on the basis of identified proteins, methods which allow the development of therapeutical active substances capable of therapeutically intervening in a pathophysiology which is caused, for example, by dysregulated expression and/or expression of nonfunctional variants but which may also appear in the case of physiological expression. It is therefore also an object of the present invention to provide corresponding substances.

We have found that this object is achieved by the subject matters of claims 1, 5, 6, 8, 11, 12, 15, 16, 17, 20, 23, 26 and 27. Advantageous embodiments are described in the relevant subclaims.

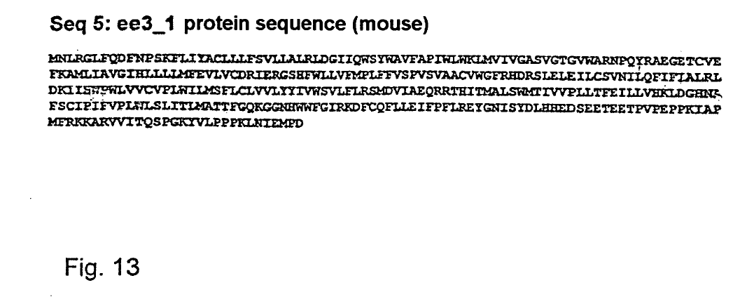

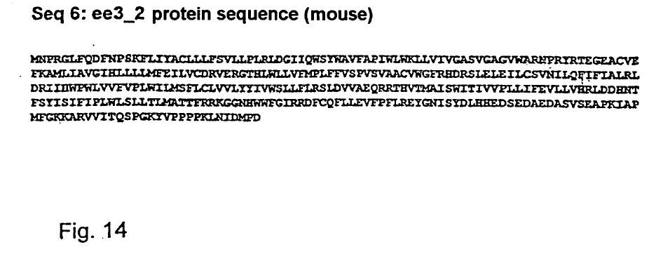

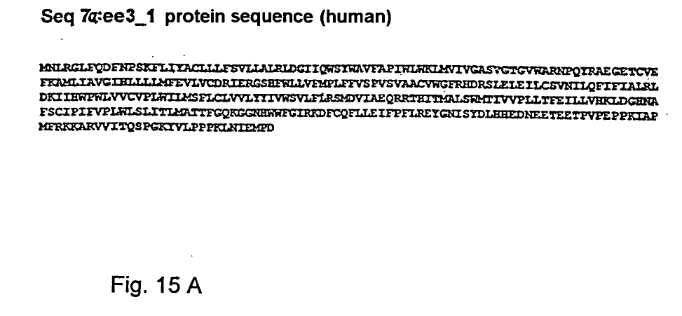

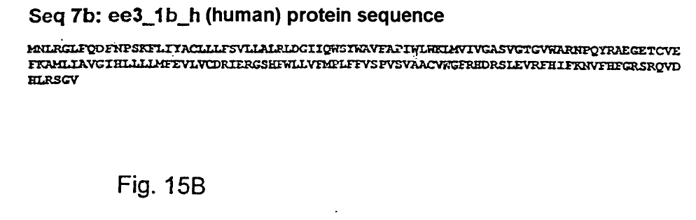

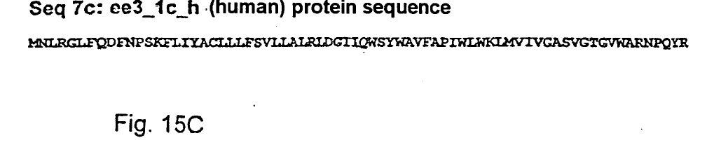

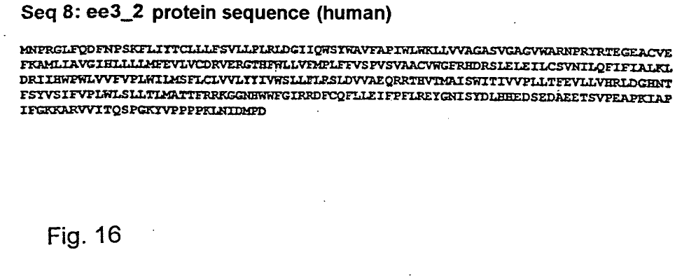

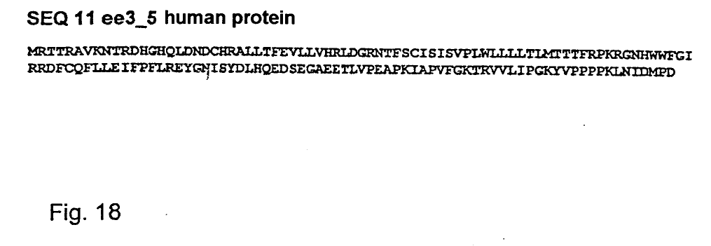

One subject matter of the present invention relates to nucleic acid sequences, in particular DNA sequences, which comprise a sequence region coding for a polypeptide with an amino acid sequence from AA 10 to AA 45 (sequence 5 according to FIG. 13, in each case referred to from N terminus to C terminus), more preferably AA 10 to 65; AA 10 to AA 45 (sequence 6 according to FIG. 14), more preferably AA 10 to 75; AA 10 to AA 45 (sequence 7A according to FIG. 15A), more preferably AA 10 to 60; AA 10 to AA 45 (sequence 7B according to FIG. 15B), more preferably AA 10 to 60, even more preferably AA 10 to AA 100; AA 10 to AA 45 (sequence 7C according to FIG. 15C), more preferably AA 10 to 60, even more preferably AA 10 to AA 100; AA 10 to AA 45 (sequence 7B according to FIG. 15B), more preferably AA 10 to 60, even more preferably AA 10 to AA 70; AA 10 to AA 45 (sequence 8 according to FIG. 16), more preferably AA 10 to 60, even more preferably AA 10 to AA 100; or AA 10 to AA 45 (sequence 11 according to FIG. 18), more preferably AA 10 to 60, even more preferably AA 10 to AA 100, including any functionally homologous derivatives, fragments or alleles. Further preference is given to those nucleic acid sequences, in particular DNA sequences, which comprise a sequence region coding for a polypeptide with an amino acid sequence of a protein of the ee3 family, and the C-terminal (intracellular) section of a protein of the invention or a fragment thereof (preferably of at least 25 AA in length), in particular, should be included. The disclosure also comprises in particular any nucleic acid sequences which hybridize with the sequences of the invention, including the sequences in each case complementary in the double strand.

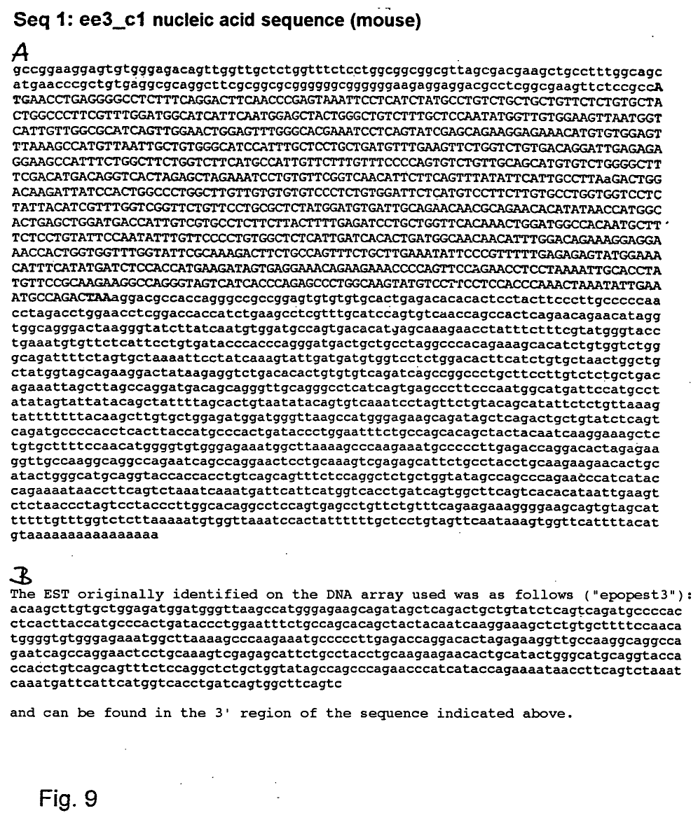

Another preferred embodiment discloses DNA sequences whose gene product codes for a polypeptide as represented in any of FIGS. 13, 14, 15A, 15B, 15C, 16 and 18 for the sequences numbers 5, 6, 7A, 7B, 7C, 8 and 11, respectively, including any functionally homologous derivatives, alleles or fragments of such a DNA sequence and also nonfunctional derivatives, alleles, analogs or fragments (e.g. DN variants) capable of inhibiting the physiological function, for example apoptotic signal cascade. Also disclosed here are DNA sequences hybridizing with said DNA sequences of the invention (including the sequences of the complementary DNA strand). Preferably, the derivatives, alleles, fragments or analogs of the AA sequences of the invention, numbers 5 to 8, or other native members of the ee3 family retain at least one biological property. Derivatives, analogs, fragments or alleles of this kind are prepared by standard methods (Sambrook et al. 1989 und 2001, Molecular Cloning: A Laboratory Manual, Cold Spring Harbor, N.Y.). To this end, one or more codons are inserted, deleted or substituted in the DNA sequences of the invention, which belong to the ee3 family, for example according to FIGS. 9, 10, 11A, 11B, 11C or 12, in order to obtain, after transcription and translation, a polypeptide which differs from the corresponding native ee3 proteins, in particular the sequences depicted in FIGS. 13, 14, 15A, 15B, 15C, 16 or 18, with respect to at least one amino acid.

The present application also relates to partial DNA sequences of the native ee3 sequences of the invention, for example the sequences depicted in FIGS. 9, 10, 11A, 11B, 11C or 12. These partial sequences typically comprise fragments of the nucleotide sequences depicted in FIGS. 9, 10, 11A, 11B, 11C or 12, which fragments comprise at least 60, more preferably at least 150, and even more preferably at least 250, nucleotides. Preferred partial sequences code in particular for polypeptides extending from AA 20 (seq. 5), AA 20 (seq. 6), AA 20 (seq. 7A), AA 20 (seq. 7B), AA 10 (seq. 7C) and, respectively, AA 20 (seq. 8) in the direction of the C terminus by at least 20 AA, preferably at least 40, more preferably at least 60, and most preferably extending to the C terminus (numbering according to FIGS. 13, 14, 15A, 15B, 15C, 16 and 18, respectively). On the other hand, the partial sequence coding for at least 20 AA may also start at a codon located more proximally or distally from the points mentioned above. Also disclosed are any derivatives, analogs or alleles of the partial sequences disclosed above. The AA sequences resulting from said partial DNA sequences of the invention are also disclosed, either by themselves or as part in larger recombinant proteins. The present disclosure also comprises in particular any conceivable or natively occurring splice variants of the sequences of the invention.

Further preference is given to nucleic acid sequences, in particular DNA sequences, which code for a protein whose sequence is at least 60%, preferably at least 80%, and even more preferably at least 95%, identical to the sequences according to the present numbering 5, 6, 7 and 8. The nucleotide sequences of the invention, for example according to FIGS. 9, 10, 11A, 11B, 11C or 12, or functional or nonfunctional equivalents thereof, such as allele variants or isoforms, for example, can be obtained after isolation and sequencing. Allele variants mean in accordance with the present invention variants which are from 60 to 100%, preferably 70 to 100%, very particularly preferably 90 to 100%, homologous at the amino acid level. Allele variants comprise in particular also those functional or nonfunctional variants which are obtainable by deletion, insertion or substitution of nucleotides from native ee3 sequences, for example from sequences depicted according to FIGS. 9, 10, 11A, 11B, 11C or 12, still retaining at least one of the essential biological properties.

Homologs or sequence-related DNA sequences may be isolated from any mammalian species or other species, including humans, according to common methods by homology screening by hybridization with a sample of the nucleic acid sequences of the invention or parts thereof. Functional equivalents also mean homologs of the native ee3 sequences, for example the sequences depicted in FIGS. 9, 10, 11A, 11B, 11C or 12, for example their homologs from other mammals, truncated sequences, single strand DNA or RNA of the coding and noncoding DNA sequence. Such functional equivalents can be isolated, for example, starting from the DNA sequences depicted in FIGS. 9, 10, 11A, 11B, 11C or 12 or parts of said sequences, from other vertebrates such as mammals, for example by usual hybridization methods or by PCR. According to the invention, this includes any sequences hybridizing with the ee3 sequences of the invention, in particular with the sequences according to FIGS. 9, 10, 11A, 11B, 11C or 12. These DNA sequences hybridize with the sequences of the invention under standard conditions. Advantageously, short oligonucleotides of the conserved regions which may be determined in a manner known to the skilled worker are used for hybridization. However, it is also possible to use longer fragments of the nucleic acids of the invention or the complete sequences for hybridization.

Said standard conditions vary depending on the nucleic acid sequence used (oligonucleotide, longer fragment or complete sequence) and/or depending on the type of nucleic acid (DNA or RNA) used for hybridization. Thus, for example, the melting temperatures for DNA:DNA hybrids are approx. 10° C. lower than those of DNA:RNA hybrids of the same length. Depending on the nucleic acid, standard conditions mean, for example, temperatures between 42 and 58° C. in an aqueous buffer solution having a concentration between 0.1 to 5×SSC (1×SSC=0.15 M NaCl, 15 mM sodium citrate, pH 7.2) or, additionally, in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids, the hybridization conditions are advantageously 0.1×SSC and temperatures between about 30° C. to 55° C., preferably between about 45° C. to 55° C. These temperatures indicated for hybridization are melting temperature values calculated, by way of example, for a nucleic acid of approx. 100 nucleotides in length and having a G+C content of 50% in the absence of formamide. The experimental conditions for DNA hybridization are described in specialist textbooks of genetics, for example in Sambrook et al. (“Molecular Cloning”, Cold Spring Harbor Laboratory, 1989), and can be calculated according to formulae known to the skilled worker, for example as a function of the length of the nucleic acids, the type of hybrids or the G+C content. The skilled worker can find further information on hybridization in the following textbooks: Ausübel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Equivalents of nucleic acid sequences of the invention include in particular also derivatives of the sequences depicted in FIGS. 9, 10, 11A, 11B, 11C or 12, such as promoter variants, for example. The promoters which are located, either together or separately, upstream of the nucleotide sequences indicated may have been altered by one or more nucleotide substitutions, by insertion(s) and/or deletion(s), it being possible to either retain or, as required, alter the functionality or efficacy of said promoters. Thus it is possible to increase the efficacy of said promoters by altering their sequence or completely replace them with more effective promoters, even from organisms of other species.

According to the invention, derivatives also mean variants whose nucleotide sequence in the region from −1 to −1000 upstream of the start codon has been altered in such a way that gene expression and/or protein expression are altered, preferably increased.

Furthermore, derivatives also mean variants which have preferably been modified at the 3′ end. Examples of such “tags” known in the literate are hexa-histidine anchors or epitopes capable of being recognized as antigens of various antibodies (for example also the flag tag) (Studier et al., Meth. Enzymol., 185, 1990: 60-89 and Ausubel et al. (eds.) 1998, Current Protocols in Molecular Biology, John Wiley & Sons, New York), and/or at least one signal sequence for transporting the translated protein, for example into a particular cell organelle or into the extracellular space.

In addition, a nucleic acid construct of the invention or a nucleic acid of the invention, for example according to FIGS. 9, 10, 11A, 11B, 11C or 12, or derivatives, variants, homologs or, in particular, fragments thereof may also be expressed in a therapeutically or diagnostically suitable form. The recombinant protein may be generated by using vector systems or oligonucleotides which extend the nucleic acids or the nucleic acid construct by particular nucleotide sequences and thus code for modified polypeptides suitable, for example, for simple purification, referring here, in particular, also to extension by the above-described tag sequences.

Preference is furthermore given to DNA sequences comprising or corresponding to (c)DNA sequences of genomic DNA sequences of the invention.

According to the invention, preference is furthermore given to disclosing any DNA sequences coding for a protein which essentially corresponds to the amino acid sequence of the inventive proteins having sequence numbers 5, 6, 7A, 7B, 7C, 8 and 11. These DNA sequences receive only a small number of modifications compared to the sequences indicated in the figures mentioned above and may be isoforms, for example. The number of sequence modifications will typically not be greater than 10. Such DNA sequences which essentially correspond to the DNA sequences coding for the proteins with the sequence numbers 5, 6, 7A, 7B, 7C, 8 and/or 11 and which likewise code for a biologically active protein may be obtained by well-known mutagenesis methods and the biological activity of the proteins encoded by the mutants may be identified by screening methods, for example binding studies or the ability to express the biological function, for example in association with neuronal processes or apoptosis. The corresponding mutagenesis methods include “site-directed” mutagenesis which involves automated synthesis of a primer with at least one base modification. After the polymerization reaction, the heteroduplex vector is transferred to a suitable cell system (e.g. E. coli) and appropriately transformed clones are isolated.

The functionality of sequences of the invention is, inter alia, directly connected with the identification of more distal elements of the signal cascade triggered by proteins of the invention. To this end, it was found according to the invention that receptors of the invention stimulate MAP kinases. Aside from using appropriate reporter assays (see exemplary embodiment) for identifying said MAP kinases, it is alternatively also possible to use prefabricated kits for these purposes (e.g. Mercury in vivo kinase assay kits from Clontech). This involves the expression of the tet repressor fused to the transactivator domain of a phosphorylation target (transcription factors, e.g. jun). Activation of a luceriferase construct under the control of a tet-repressor element takes place only if the transactivator domain is specifically phosphorylated by a kinase. In this way it is possible, according to the invention, to assign the activity of an inventive receptor of the ee3 family or of an inventive variant to a cellular signal transduction pathway.

The identification of sequences of the invention is based, inter alia, also on the functional finding that upregulation of murine ee3_{—}1_m of the invention in an animal model with increased EPO expression indicates a pathophysiological involvement of said receptor in processes influencing cell survival or cell adaptation to this state. Therefore, receptors of the invention are of particular pharmacological importance for diseases accompanied by reduced oxygen supply, in particular reduced cerebral oxygen supply.

In addition, any methods familiar to the skilled worker for preparation, modification and/or detection of DNA sequences of the invention are suitable that can be carried out in vivo, in situ or in vitro (PCR (Innis et al. PCR Protocols: A Guide to Methods and Applications) or chemical synthesis). Appropriate PCR primers can introduce, for example, new functions into a DNA sequence of the invention, such as, for example, restriction cleavage sites, termination codons. This makes it possible to correspondingly design sequences of the invention for transfer into cloning vectors.

The present invention furthermore relates to expression vectors or to a recombinant nucleic acid construct which comprises a nucleic acid sequence of the invention, as described above, typically a DNA sequence. Advantageously, the nucleic acid sequences of the invention are functionally linked here to at least one genetic regulatory element such as transcription and translation signals, for example. Depending on the desired application, this linkage may result in a native rate of expression or else in an increase or reduction in native gene expression. The expression vectors prepared in this way may then be used for transforming host organisms or host cells, for example cell cultures of mammalian cells.

In the expression vector of the invention, the native regulatory element(s) will typically be used, i.e., for example, promoter and/or enhancer region of the gene for an inventive protein of the ee3 family, in particular for a protein with sequence number 5, 6, 7A, 7B, 7C, 8 or 11, for example from mammals, in particular corresponding human regulatory sequences. These native regulatory sequences indicated above may, where appropriate, also be genetically modified in order to cause an altered expression intensity. In addition to said native regulatory sequences indicated above or instead of said native regulatory sequences, it is possible for other genes to have native regulatory elements upstream and/or downstream of DNA sequences of the invention (5′ or 3′ regulatory sequences), which may also, where appropriate, have been genetically modified so that natural regulation under the control of the native regulatory sequences indicated above is switched off, thereby enabling expression of said genes to be increased or reduced, as desired.

Advantageous regulatory sequences of the method of the invention are present, for example, in promoters such as cos, tac, trp, tet, trp-tet, lpp, lac, lpp-lac, lacIq, T7, T5, T3, gal, trc, ara, SP6, l-PR or in the 1-PL promoter, which promoters are advantageously applied in Gram-negative bacteria. Further advantageous regulatory sequences are present, for example, in Gram-positive promoters such as amy and SPO2, in yeast promoters such as ADC1, MFa, AC, P-60, CYC1, GAPDH or in mammalian promoters such as CaM KinaseII, CMV, nestin, L7, BDNF, NF, MBP, NSE, beta-globin, GFAP, GAP43, tyrosine hydroxylase, kainate-receptor subunit 1, glutamate-receptor subunit B. In principle, any natural promoters with their regulatory sequences such as those mentioned above, for example, may be used for an expression vector of the invention.

In addition, it is also possible and advantageous to use synthetic promoters. These regulatory sequences are intended to enable targeted expression of the nucleic acid sequences of the invention. Depending on the host organism, this may mean, for example, that the gene is expressed or overexpressed only after induction or that it is expressed and/or overexpressed immediately. The regulatory sequences or factors may preferably have a beneficial influence on and thereby increase expression. Thus the regulatory elements may advantageously be enhanced at the transcriptional level by using strong transcription signals such as promoters and/or enhancers. In addition, however, it is also possible to enhance translation by improving the stability of mRNA, for example.

Regulatory sequences refer to any elements familiar to the skilled worker which are capable of influencing expression of the sequences of the invention at the transcriptional and/or translational level. Besides promoter sequences, particular emphasis must be placed on “enhancer” sequences which are capable of increasing expression via an improved interaction between RNA polymerase and DNA. Further regulatory sequences which may be mentioned by way of example, are the “locus control regions”, “silencers” or particular partial sequences thereof. These sequences may advantageously be used for tissue-specific expression. Advantageously, an expression vector of the invention will also contain “terminator sequences” which are subsumed according to the invention under the term “regulatory sequence”.

A preferred embodiment of the present invention is linkage of the nucleic acid sequence of the invention to a promoter, said promoter typically being located 5′ upstream of a DNA sequence of the invention. Further regulatory signals such as, for example, 3′ terminators, polyadenylation signals or enhancers may be functionally present in the expression vector. In addition, one or more copies of nucleic acid sequences of the invention, in particular for the sequences according to FIGS. 9, 10, 11A, 11B, 11C or 12 or for the corresponding proteins, may be present in a gene construct according to the present invention or, where appropriate, also on separate gene constructs.

The term “expression vector” includes both recombinant nucleic acid constructs or gene constructs, as described previously, and complete vector constructs which typically also contain further elements in addition to DNA sequences of the invention and possible regulatory sequences. These vector constructs or vectors are used for expression in a suitable host organism. Advantageously, at least one DNA sequence of the invention, for example human gene of the ee3 family, in particular ee3_{—}1 or ee3_{—}2, or, for example, a partial sequence of such a gene is inserted into a host-specific vector which enables the genes to be optimally expressed in the selected host. Vectors are well known to the skilled worker and can be found, for example, in “Cloning Vectors” (Eds. Pouwels P. H. et al. Elsevier, Amsterdam-New York-Oxford, 1985, ISBN 0 444 904018). Vectors mean, in addition to plasmids, also any other vectors known to the skilled worker, such as, for example, phages, viruses such as SV40, CMV, baculovirus, adenovirus, Sindbis virus, transposons, IS elements, phasmids, phagemids, cosmids, linear or circular DNA. Said vectors can replicate autonomously in the host organism or chromosomally. Integration into Mammalia typically uses linear DNA.

Advantageously, expression of nucleic acid sequences of the invention can be increased by increasing the number of gene copies and/or by enhancing regulatory factors having a beneficial influence on gene expression. Thus it is possible to enhance regulatory elements preferably at the transcriptional level by using stronger transcription signals such as promoters and enhancers. Aside from this, however, it is also possible to enhance translation by improving, for example, the stability of mRNA or increasing the reading efficiency of said mRNA on the ribosomes. The number of gene copies can be increased by incorporating the nucleic acid sequences or homologous genes, for example, into a nucleic acid fragment or into a vector which preferably contains the regulatory gene sequences assigned to the particular genes or promoter activity acting in a similar manner. Use is made in particular of those regulatory sequences which enhance gene expression.

Nucleic acid sequences of the invention may be cloned together with the sequences coding for interacting or for potentially interacting proteins into a single vector and subsequently be expressed in vitro in a host cell or in vivo in a host organism. Alternatively, it is also possible to introduce each of the potentially interacting nucleic acid sequences and the inventive coding sequences of the ee3 family in each case into a single vector and to transport said vectors separately into the particular organism via usual methods such as, for example, transformation, transfection, transduction, electroporation or particle gun.

In another advantageous embodiment, at least one marker gene (e.g. antibiotic resistance gene and/or genes coding for a fluorescent protein, in particular GFP) may be present in an expression vector of the invention, in particular in a complete vector construct.

The present invention further relates to host cells transformed with a DNA sequence of the invention and/or an expression vector of the invention, in particular a vector construct. Suitable host cells are in principle any cells which allow DNA sequences of the invention (which include as a result, for example, as derivatives also their alleles or functional equivalents) to be expressed alone or associated with other sequences, in particular regulatory sequences. Suitable host cells are all pro- or eukaryotic cells, for example bacteria, fungi, yeasts, plant or animal cells. Preferred host cells are bacteria such as Escherichia coli, Streptomyces, Bacillus or Pseudomonas, eukaryotic microorganisms such as Aspergillus or Saccharomyces cerevisiae or common baker's yeast (Stinchcomb et al., Nature, 282:39, (1997)). Methylotrophic yeasts, in particular Pichia pastoris, are particularly and advantageously suitable for being able to prepare relatively large amounts of proteins of the invention. For this purpose, the receptors are cloned into suitable expression vectors which allow, for example, also expression as fusion protein containing tag sequences useful for purification. Finally, after electroporation of the yeasts, stable clones are selected. The company Invitrogen offers a good description of the method and all means required therefor. The expression products may thereafter be functionally characterized and, where appropriate, used for screening methods of the invention.

In a preferred embodiment, however, cells from multicellular organisms are chosen for expression of DNA sequences of the invention. This takes place also against the background of a possibly required glycosylation (N-and/or O-coupled) of the encoded proteins. In contrast to prokaryotic cells, higher eukaryotic cells are able to carry out this function in a suitable manner. In principle, any higher eukaryotic cell culture is available as a host cell, albeit very particular preference being given to cells of mammals, for example monkeys, rats, hamsters or humans. A multiplicity of established cell lines is known to the skilled worker. The following cell lines are mentioned, the list being by no means complete: 293T (embryonic kidney cell line), (Graham et al., J. Gen. Virol., 36:59 (1997)), BHK (baby hamster kidney cells), CHO (hamster ovary cells), (Urlaub und Chasin, P. N. A. S. (USA) 77:4216, (1980)), HeLa (human cervical carcinoma cells) and other cell lines, in particular those established for laboratory use, such as, for example, CHO, HeLa, HEK293, Sf9 or COS cells. Very particular preference is given to human cells, in particular cells of the immune system or adult stem cells, for example stem cells of the hematopoietic system (from bone marrow). Human transformed cells of the invention, in particular autologous cells of the patient, are, after (especially ex vivo) transformation with DNA sequences of the invention or expression vectors of the invention, very particularly suitable as drugs for the purposes of, for example gene therapy, i.e. after removal of cells, where appropriate ex vivo expansion, transformation, selection and final retransplantation.

Particularly advantageous according to the invention will be the heterologous production of inventive proteins of the ee3 family in insect cells for functional characterization and for use in screening methods of the invention. Since the concentration of endogenous G proteins in insect cells is relatively low, meaning, for example, that Gi proteins cannot be detected in a Western blot, and since insect cells do normally not express the receptor to be studied, said cells are particularly suitable for in vivo reconstitution of signal transduction pathways of inventive receptors of the ee3 family. In this case, the receptors of the ee3 family are expressed by means of the baculovirus expression system in various insect cell lines, for example Sf9, Sf21, Tn 368 or Tn High Five, or MB cells. For this purpose, recombinant baculoviruses are prepared using, for example, the BaculoGold Kit from Pharmingen and the above-mentioned insect cell lines are infected. In order to study according to the invention coupling to G proteins, coinfections are carried out. For this purpose, the cells are infected with the receptor virus and, in addition, also with the viruses expressing the three G protein subunits, and corresponding assays, for example cAMP assays, are carried out. Thus it is possible to study the influence of various G protein subunits on the activity of the receptor. Insect cells expressing the receptors or their membranes may likewise be used in screening assays. Insect cells can be readily propagated in large amounts either in fermenters or in shaker flasks and are thus a suitable starting material in order to provide recombinant cell or membrane material both for screening methods and for receptor purifications.

The combination of a host cell and an inventive expression vector suitable for the host cells, such as plasmids, viruses or phages, for example plasmids containing the RNA polymerase/promoter system, the phages 1, mu or other temperate phages or transposons, and/or other advantageous regulatory sequences produces a host cell of the invention, which may serve as expression system. Preferred examples of expression systems of the invention based on host cells of the invention are the combination of mammalian cells such as, for example, CHO cells and vectors such as, for example, pcDNA3neo vector, or, for example, HEK293 cells and CMV vector which are particularly suitable for mammalian cells.

Another aspect of the present invention relates to the gene products of the DNA sequences of the invention. Gene products mean in accordance with this invention both primary transcripts, i.e. RNA, preferably mRNA, and proteins and/or polypeptides, in particular in purified form. These proteins regulate or transport in particular apoptotic or necrotic, where appropriate also inflammatory, signals or signals relating to cell growth or cell plasticity. Preference is given to a purified gene product if it comprises a functionally homologous or function-inhibiting (nonfunctional) allele, fragment, analog or derivative of this sequence or typically consists of such an amino acid sequence. In accordance with the present invention, functional homology is defined in such a way that at least one of the essential functional properties of the protein depicted according to FIGS. 13 to 16 and/or 18, whose sequences are denoted 5, 6, 7a (including 7b and 7c), 8 and 11, is retained. Typically, functionally homologous proteins of the invention will have sequences which are in particular and characteristically, for example at least 60%, preferably at least 80%, identical to the biologically functional sections of the proteins of the invention, which are protein interaction domains, for example. According to the present invention, the disclosure also includes in particular the homologous sequences on chromosomes 3, 5, 8 and X, disclosed according to exemplary embodiment 6, and also their variants.

Derivative here means in particular also those AA sequences which have been altered by modification of their side chains, for example by conjugation of an antibody, enzyme or receptor to an AA sequence of the invention. Derivatives may, however, also coupling of a sugar (via an N- or O-glycosidic bond) or fatty (acid) residue (e.g. myristic acid), of one or else more phosphate groups and/or any modification of a side chain, in particular of a free OH group or NH2 group or on the N or C terminus of an oligo- or polypeptide of the invention. In addition, the term “derivative” also includes fusion proteins, i.e. proteins in which an amino acid sequence of the invention is coupled to any oligo- or polypeptides.

“Analogs” refer to sequences which are distinguished by at least one AA modification compared to the native sequence (insertion, substitution). For the purposes of the present invention, preference is given to those conservative substitutions which retain the physico-chemical character (bulk, basicity, hydrophobicity etc.) of the substituted AA (polar AA, long aliphatic chain, short aliphatic chain, negatively or positively charged AA, AA with aromatic group). The substitutions may result in biologically functional, partially functional or biologically nonfunctional sequences. For example, lysine residues may be substituted for arginine residues, isoleucine residues for valine residues or glutamic acid residues for aspartic acid residues. It is, however, also possible to add or remove or to change the order of one or more amino acids or to combine several of these measures with one another. The proteins altered in this way compared to the native ee3 proteins, in particular compared to FIGS. 13, 14, 15A, 15B, 15C, 16 or 18, typically have sequences which are at least 60%, preferably at least 70%, and particularly preferably at least 90%, identical to the sequences in the above-mentioned figures, calculated according to the algorithm by Altschul et al. (J. Mol. Biol., 215, 403-410, 1990). The isolated protein and its functional variants can advantageously be isolated from the brain of Mammalia such as Homo sapiens, Rattus norvegicus or Mus musculus. Functional variants mean also homologs from other Mammalia.

According to the invention, preference is given to analogs if they also retain the secondary structure as it appears in the native sequence. It is also possible to introduce according to the invention less conservative AA variations into the native sequence, in addition to conservative substitutions. The former typically retain their biological function here, in particular as transducer of an apoptotic or necrotic signal or of a signal for cell proliferation, cell plasticity or cell growth. The effect of a substitution or deletion can be readily tested by way of appropriate studies, binding assays or cytotoxic assays, for example.

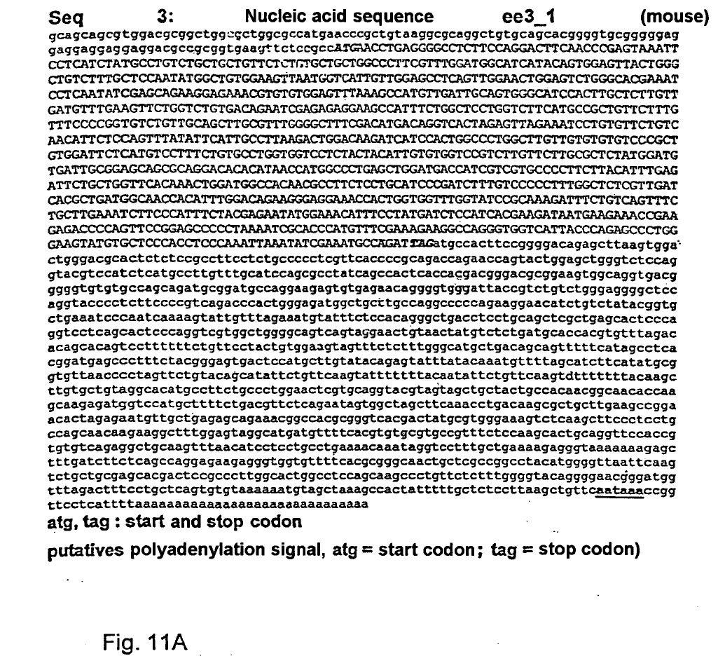

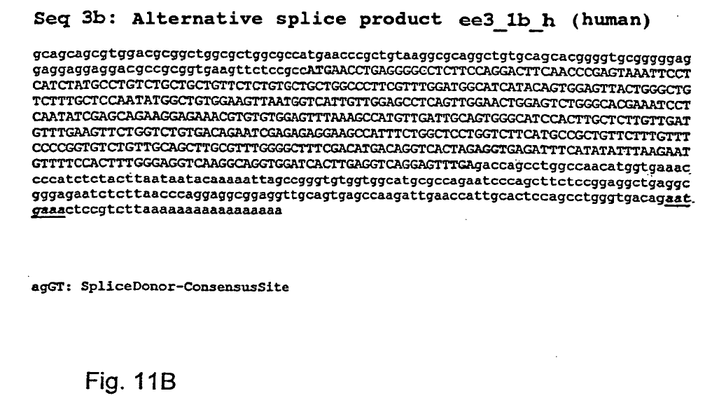

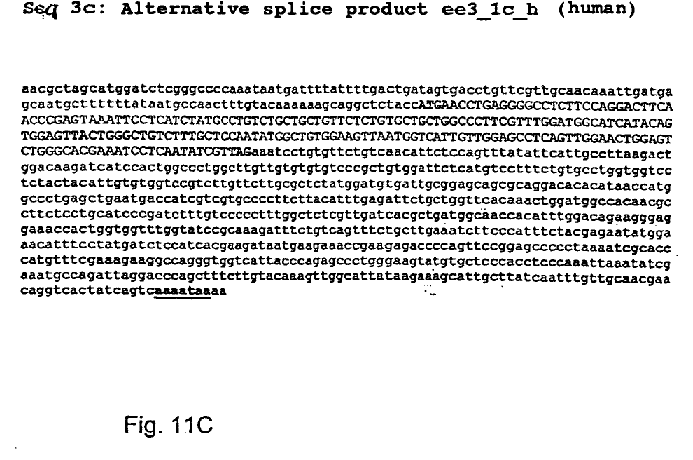

Nonetheless, however, the invention also includes sequences which are capable of causing a “dominant negative” effect, i.e. sequences which, due to their altered primary sequence, still have binding activity to an extracellular ligand but are unable to pass on the signal downstream, i.e. intracellularly. Examples which may be disclosed here are variants of an ee3_{—}1 sequence whose C terminus is truncated, for example also the two splice variants according to FIGS. 11B or 11C and, respectively, 15B or 15C. Analogs of this kind therefore act as inhibitors of the biological function, in particular as inhibitors of apoptosis. Analogs of this kind are genetically engineered, typically by “site-directed” mutagenesis of a DNA sequence coding for a protein of the invention (typically sequences numbered 5, 6, 7(b,c), 8 and 11). This produces the DNA sequence on which the analog is based and which can ultimately express the protein in a recombinant cell culture (Sambrook et al., 1989, see above). Any derivatives of the above-described analogs as well as the DNA sequences on which the above-described AA sequences are based are also disclosed.

The present invention furthermore also includes fragments of a native AA sequence of the invention. Fragments are distinguished by deletions (N- or C-terminally or else intrasequentially). They may have a dominant-negative or dominant-positive effect.

However, the gene products (proteins) of the invention also include all those gene products (proteins) which derive according to the invention from DNA derivatives, DNA fragments or DNA alleles of the DNA sequences indicated in the figures, after transcription and translation.

In addition, the proteins of the invention may be chemically modified. Thus, for example, a protective group may be present on the N terminus. Glycosyl groups may be attached to hydroxyl or amino groups, lipids may be covalently linked to the protein of the invention, likewise phosphates or acetyl groups and the like. Any chemical substances, compounds or groups may also be bound to the protein of the invention via any synthetic route. Additional amino acids, for example in the form of individual amino acids or in the form of peptides or in the form of protein domains and the like, may also with the N- and/or C-terminus of a protein of the invention.

Particular preference is given here to “signal” or “leader” sequences on the N-terminus of the amino acid sequence of a protein of the invention, which guides the peptide cotranslationally or posttranslationally to a particular cell organelle or into the extracellular space (or culture medium). Amino acid sequences which allow, as an antigen, the amino acid sequence of the invention to bind to antibodies may also be present at the N or at the C terminus. Particular mention must be made here of the Flag peptide whose sequence, in the one-letter amino acid code, is as follows: DYKDDDDK. Or else a His tag having at least 3, preferably at least 6, histidine residues. These sequences have strongly antigenic properties and thus allows rapid testing and simple purification of the recombinant protein. Monoclonal antibodies binding the Flag peptide are available from Eastman Kodak Co., Scientific Imaging Systems Division, New Haven, Conn.

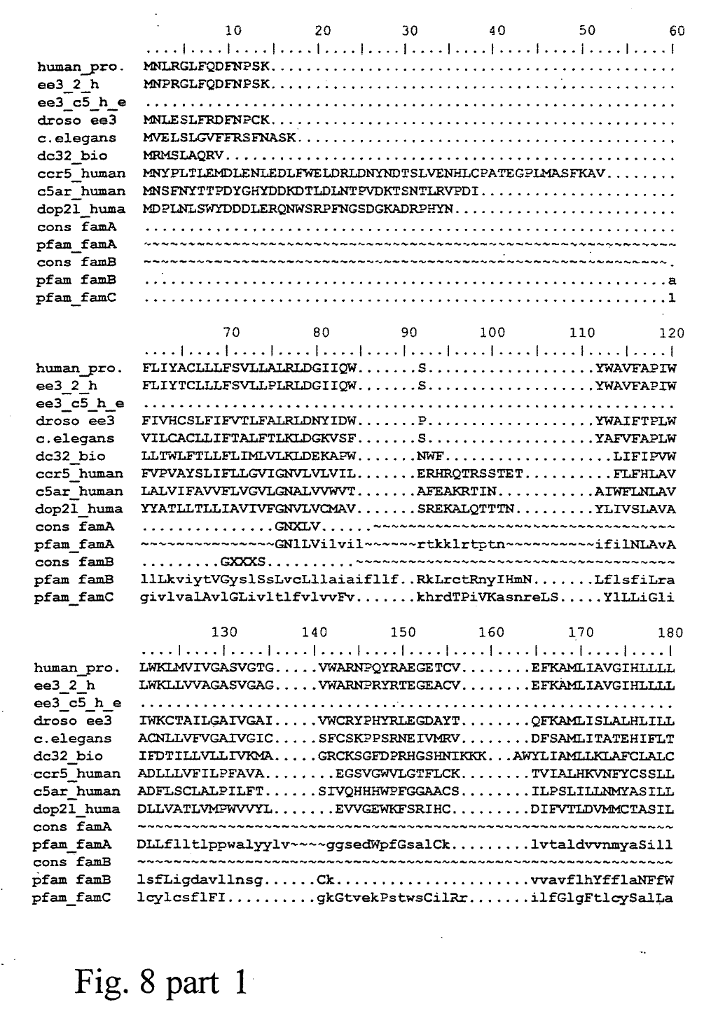

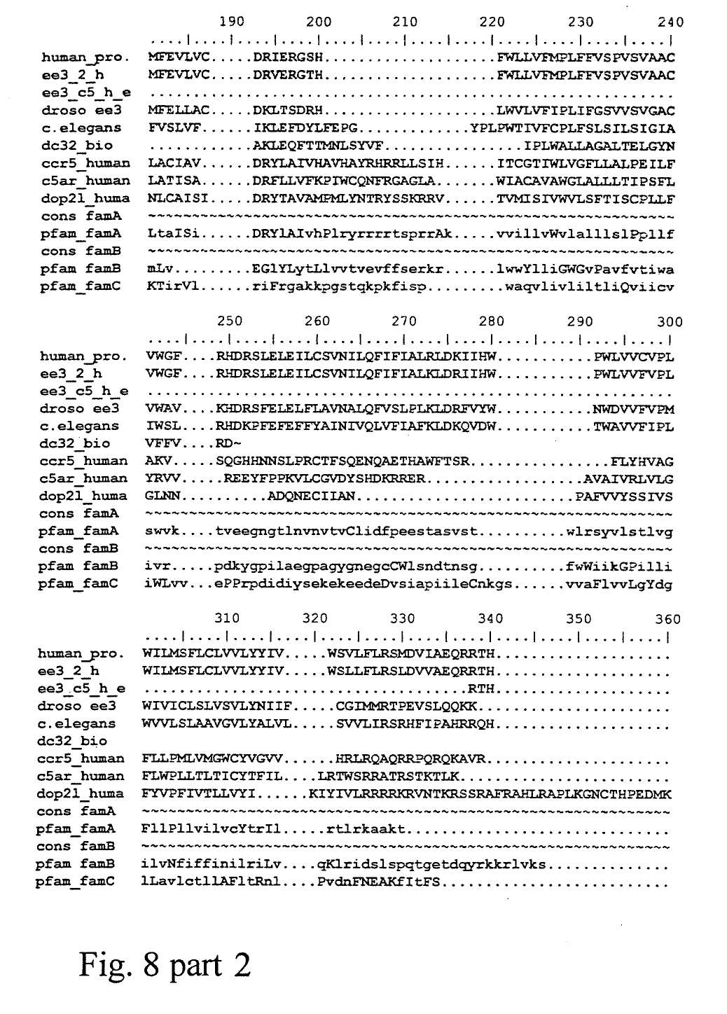

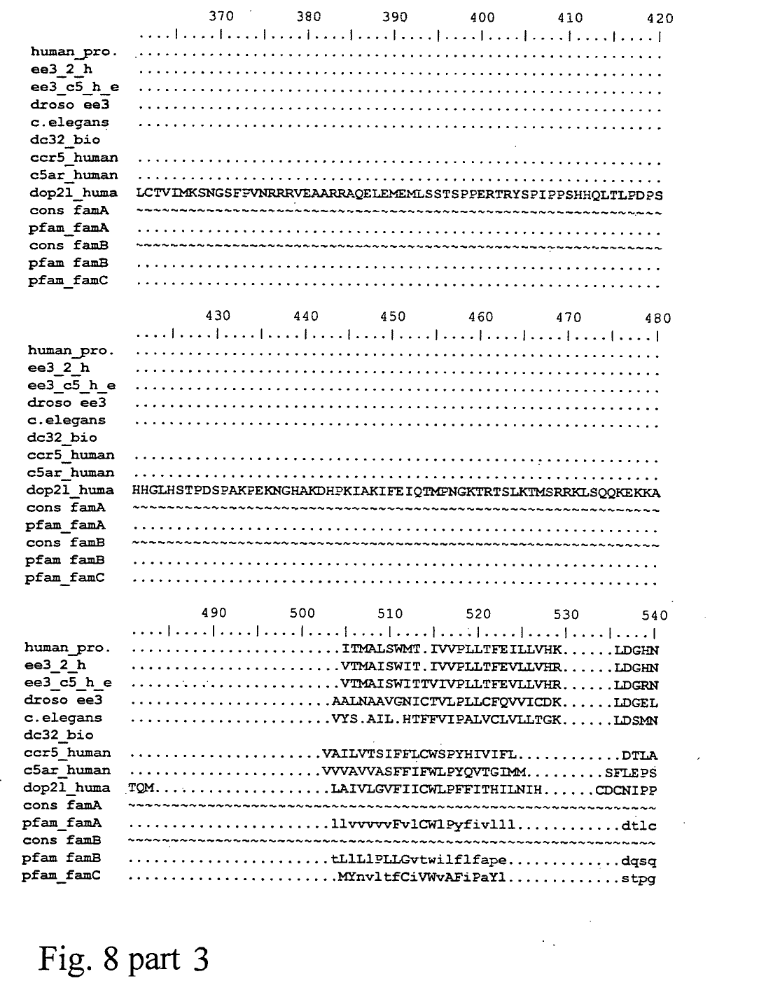

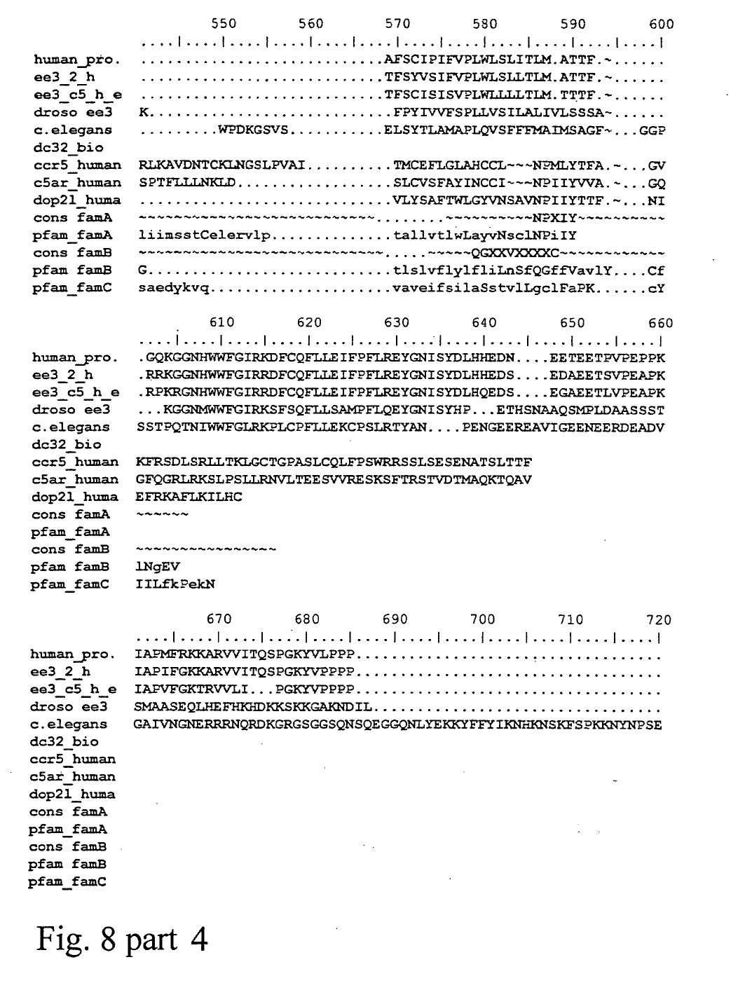

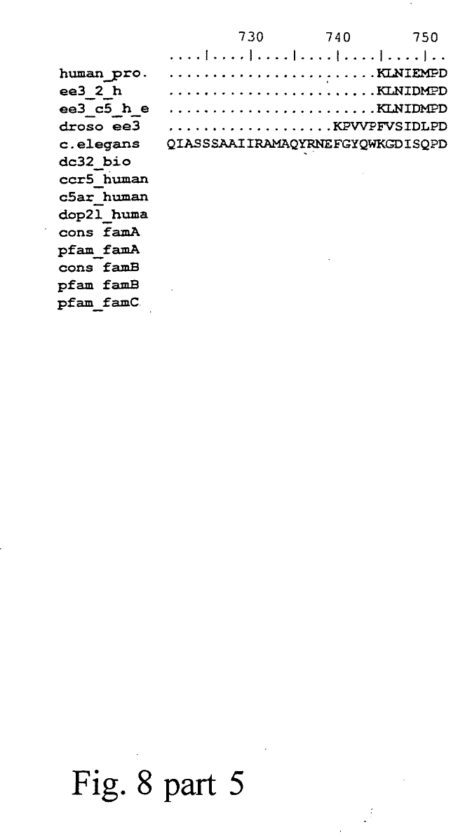

The present invention further relates to sections of the native ee3 sequences, in particular of the sequences as disclosed in FIGS. 13, 14, 15A, 15B, 15C, 16 or 18, which sections comprise at least 20, more preferably at least 30 and even more preferably at least 50 amino acids. Partial sequences of this kind may be chemically synthesized according to methods known to the skilled worker, for example, and preferably be used as antigens for producing antibodies. Preferably, these sections and/or derivatives, alleles or fragments thereof will be disclosed sequences which, in the three dimensional model of the proteins, form those regions which, at least partially, make up the protein surface in the native ee3 sequences of the invention, in particular those in FIGS. 13, 14, 15A, 15B, 15C, 16. Preferred partial sequences of at least 20 AA in length will comprise, at least partially, the cytoplasmic section of the proteins of the invention, and, particularly preferably, a section of the invention will have peptides of at least 20 AA in length of any of the sequences of the invention according to FIG. 8, between position 600 and position 752 (according to FIG. 8), for example the peptide WWFGIRKDFCQFLLEIFPFLRE (positions 609 to 630, length: 21 AA).

AA sequences of the invention, for example the sequences of the human proteins ee3_{—}1 or ee3_{—}2, in addition have specific sequence motifs which can also be found in a similar form in other representatives of the GPCR class. Thus, for example, a typical signature triplet sequence appears downstream of the third transmembrane domain in GPCR class proteins (sequence containing the sequence DRY (AA in one-letter code).

In ee3_{—}1 of the invention, the sequence DRI (positions 103-105) can be found downstream of TM3 (83-102) (according to FIG. 15A). In the GPCR class representatives known according to the prior art (galanine-2 receptor, C5a receptor (rat), BK-2 (human) or CXCR-5 (human)), the sequence DRY or DRF can be found in corresponding positions.

Therefore, very particular preference is given to peptides of the invention, as described above, of at least 20 AA in length, if they encompass the AA triplet DRI. An example of an inventive peptide of this kind which may be mentioned here is a peptide having the sequence VLVCDRIERGSHFWLLVFMP. Inventive peptides of this kind may be used in particular in connection with modulating the physiological function of the receptors. In the case of incorporation or general availability of such peptide sequences in a cell, agonist-dependent activation of intracellular signal transduction processes, activation of the interaction of receptors of the invention with G proteins and, where appropriate, receptor internalization may be influenced. It is also possible, where appropriate, for certain oligo- or polypeptides of the invention to contribute to constitutive activation of the downstream signal transduction pathway. Oligo- or polypeptides of the invention are therefore very particularly suitable for use as or for the preparation of a drug.

In addition, the first two extracellular loops of oligo- or polypeptides of the invention of at least 20 AA in length, for example ee3_{—}1 (see FIG. 8), may preferably also comprise 2 conserved cysteines (positions 78 and 145 in ee3_{—}1, according to FIG. 15A) which are a typical feature of the class of GPCR proteins (sequence GETCV at the end of the first extracellular loop, sequence ELEILCSVNIL in the center of the second extracellular loop). The sequences of the oligopeptides of the invention therefore include, for example, the two abovementioned sequences.

Finally, very particular preference is also given to those peptides of at least 20 AA in length from a sequence of a protein of the ee3 family, which are from the TM regions, for example the peptide LDGHNAFSCIPIFVPLWLSLIT (partially comprising the C-terminal TM domain). Inventive peptides of this kind are preferably used for modulation, in particular inhibition, of the receptor action of ee3 proteins, the therapeutic profile of such peptides applying to the inventive indications mentioned below. Peptides inhibiting the TM structures cause a functional change, especially functional losses, in the receptors, due to disruption of normal binding. In this context, reference is made for example to corresponding approaches carried out for the sixth TM domain of the beta-2-adrenergic receptor (Hebert T E, Moffett S, Morello J P, Loisel T P, Bichet D G, Barret C, Bouvier M (1996) A peptide derived from a beta2-adrenergic receptor transmembrane domain inhibits both receptor dimerization and activation. J Biol Chem 271:16384-16392). In addition, the invention provides ligand-binding peptide fragments of at least 20 AA in length from a sequence of a protein of the ee3 family, also for use as or for the preparation of a drug, which fragments can compete with native (extra- or intracellular ligands) for binding sites and in this way can block the binding of native ee3 ligands. Inventive peptides of this type then appear as “decoy receptors”, resulting in a therapeutic profile in all indications mentioned in the present application.

Disclosure is furthermore also made of methods for identifying inhibitory peptides of the invention, for example. Suitable for this, according to the invention, is in particular the method described by Tarasova et al. in a different context (Tarasova N I, Rice W G, Michejda C J (1999) Inhibition of G protein-coupled receptor function by disruption of transmembrane domain interactions. J Biol Chem 274:34911-34915), which is in its entirety incorporated in the present disclosure, with respect to the methodical procedure. These inhibitory peptides of the invention are capable of modulating, for example inhibiting, for example an intramolecular interaction of different TM domains, an important precondition for the functioning of a protein of the invention of the ee3 family. Inventive peptides of this kind are also suitable as drugs or for preparing a drug.

Inventive peptides of the abovementioned type may also be present in the form of peptide analogs (peptidomimetics). In this case, the amide-like bond of the backbone is preferably substituted by alternative, but structurally comparable, bonds which would preferably not be cleavable by native human enzymes. Suitable here are oligocarbamates, for example. Monomeric N-protected aminoalkyl carboxylates can be readily prepared, for example, from the corresponding amino alcohols and, after conversion to activated esters using the base-labile Fmoc group, may be introduced to solid phase synthesis. Since analogs of this kind are more hydrophobic than the corresponding peptides, they are particularly suitable for overcoming the blood-brain barrier, i.e. in particular as drugs for neurological use.

The present invention further relates to transgenic animals. Transgenic animals of the invention are animals which are genetically modified so as to express or contain, in comparison to a normal animal, an altered amount of a gene product of the invention in at least one tissue (for example by way of modification of the promoter region of a gene of the invention) or which contain or express a modified gene product (for example an inventive derivative of a protein of the ee3 family, for example also a fragment). This includes according to the invention also those animals which (a) no longer have either part of or the complete natively present DNA sequence of the invention at the genetic level or which (b) still have sequences of the invention at the genetic level but cannot transcribe and/or translate said sequences and therefore no longer contain the gene product. In addition, the native sequences of the invention in a transgenic animal, i.e. sequences of the ee3 protein family, for example sequences numbered 5, 6, 7 (including 7b and 7c), 8 and 11) (whether present or not present), may be expanded by at least one DNA sequence of the invention and/or substituted by at least one DNA sequence of the invention. The substituted and/or inserted sequence(s) may be in particular normative sequences of the invention.

The preparation of animals transgenic with respect to sequences of the invention and/or of “knockout” animals, in particular mice, rats, pigs, cattle, sheep, fruit flies (Drosophila), C. elegans or zebra fish, is carried out in a manner familiar to the skilled worker. To this end, a cDNA sequence of the invention, for example, or a native or normative variant is expressed in transgenic mice, for example under an NSE promoter in neurons, under an MBP promoter in oligodendrocytes, etc. The genetically modified animals may then be studied in different disease models (e.g. experimentally caused stroke, MCAO). The preparation of knockout animals may moreover provide information on the effects of inhibitors on the entire organisms, since a “knockout model” in this respect corresponds to the inhibition of native sequences of the invention. In this respect, a method of this kind may be used in preclinical testing of inhibitory substances of the invention, for example peptides of the invention, peptide analogs or other small organic compounds.

According to the invention, all of the multicellular organisms may be designed transgenically, in particular mammals, for example mice, rats, sheep, cattle or pigs. Transgenic plants are also conceivable in principle. The transgenic organisms may also be “knockout” animals. In this context, the transgenic animals may contain a functional or nonfunctional nucleic acid sequence of the invention or a functional or nonfunctional nucleic acid construct alone or in combination with a functional or nonfunctional sequence coding for proteins of the invention.

A further inventive embodiment of the above-described transgenic animals are transgenic animals in whose germ cells or in all or some of whose somatic cells or in whose germ cells or in all or some of whose somatic cells the native inventive nucleotide sequence(s) ee3 family, in particular the sequences with numbers 1 to 4), have been altered by genetic methods or interrupted by inserting DNA elements. Another possible use of a nucleotide sequence of the invention or parts thereof is the generation of transgenic or knockout animals or of conditional or region-specific knockout animals or of specific mutations in genetically modified animals (Ausubel et al. (eds.) 1998, Current Protocols in Molecular Biology, John Wiley & Sons, New York und Torres et al., (eds.) 1997, Laboratory protocols for conditional gene targeting, Oxford University Press, Oxford). In addition, it is also possible to introduce particular mutations, for example modifications of the promoters or insertion of enhancers, in order to generate, for example, constitutively active ee3 proteins in the transgenic animals (“knock-in” animals). Such animals may also be used according to the invention, for example, in order to provide analogy models for potential agonists of the ee3 protein function in preclinical studies.

It is possible to generate, by way of transgenic overexpression or genetic mutation (null mutation or specific deletions, insertions or modifications) by homologous recombination in embryonic stem cells, animal models which provide valuable further information on the (patho)physiology of the sequences of the invention. Animal models prepared in this way may be essential test systems for evaluating novel therapeutics which influence the biological function of proteins of the invention, in particular of proteins having any of the sequences 5 to 8 for neural, immunological, proliferative or other processes.

The present invention further relates to an antibody which recognizes an epitope on an ee3 gene product of the invention, in particular on an inventive protein according to FIGS. 13, 14, 15A, 15B, 15C, 16 or 18 or derivatives, fragments or isoforms or alleles, but which may also be directed against mRNA of the invention, for example. The term “antibody” encompasses in accordance with the present invention both polyclonal antibodies and monoclonal antibodies, chimeric antibodies, anti-idiotypic antibodies (directed against antibodies of the invention), all of which may be present in bound or soluble form and, where appropriate, labeled by labels, and also fragments of said antibodies. In addition to the fragments of antibodies of the invention in isolation, antibodies of the invention may also appear in recombinant form as fusion proteins with other (protein) components. Fragments as such or fragments of antibodies of the invention as part of fusion proteins are typically prepared by the methods of enzymic cleavage, protein synthesis or the recombination methods familiar to the skilled worker. Thus, according to the present invention, polyclonal, monoclonal, human or humanized or recombinant antibodies or fragments thereof, single chain antibodies or else synthetic antibodies are referred to as antibodies.

Polyclonal antibodies are heterogeneous mixtures of antibody molecules, which are prepared from the sera of animals which have been immunized with an antigen. However, the invention also includes polyclonal monospecific antibodies obtained after purification of the antibodies (for example via a column charged with peptides of a specific epitope). A monoclonal antibody comprises an essentially homogeneous population of antibodies specifically directed against antigens and having essentially the same epitope-binding sites. Monoclonal antibodies may be obtained by the methods known in the prior art (e.g. Köhler and Milstein, Nature, 256, 495-397, (1975); U.S. Pat. No. 4,376,110; Ausübel et al., Harlow und Lane “Antikörper” [Antibodies]: Laboratory Manual, Cold Spring, Harbor Laboratory (1988); Ausubel et al., (eds), 1998, Current Protocols in Molecular Biology, John Wiley & Sons, New York).). The description found in the references above is hereby incorporated as part of the present invention into the disclosure of the present invention.

It is also possible to prepare genetically engineered antibodies of the invention by methods as described in the abovementioned applications. Briefly, said preparation involves growing antibody-producing cells and, after said cells have reached an adequate optical density, mRNA is isolated from said cells in a known manner via cell lysis with guanidinium thiocyanate, acidifying with sodium acetate, extraction with phenol, chloroform/isoamyl alcohol, precipitations with iso-propanol and washing with ethanol. Subsequently, cDNA is synthesized from said mRNA with the aid of reverse transcriptase. The synthesized cDNA may then, either directly or after genetic manipulation, for example by site-directed mutagenesis, introduction of insertions, inversions, deletions or base substitutions, be inserted into suitable animal, fungal, bacterial or viral vectors and expressed in the corresponding host organisms. Preference is given to bacterial or yeast vectors such as pBR322, pUC18/19, pACYC184, lambda or yeast mu vectors for cloning of the genes and expression in bacteria such as E. coli or in yeast such as Saccharomyces cerevisiae. Specific antibodies against the proteins of the invention may be useful both as diagnostic reagents and as therapeutics for disorders in which proteins of the ee3 family are pathophysiologically important.

Antibodies of the invention may belong to any of the following classes of immunoglobulins: IgG, IdD, IgM, IgE, IgA, GILD and, where appropriate, to a subclass of said classes, such as meaning the subclasses of IgG or mixtures thereof. Preference is given to IgG and its subclasses such as, for example, IgG1, IgG2, IgG2a, IgG2b, IgG3 or IgGM. Particular preference is given to the IgG subtypes IgG1/k or IgG2b/k. A hybridoma cell clone producing monoclonal antibodies of the invention may be cultured in vitro, in situ or in vivo. High titers of monoclonal antibodies are preferably produced in vivo or in situ.

The chimeric antibodies of the invention are molecules comprising various parts derived from various animal species (e.g. antibodies having a variable region derived from a murine monoclonal antibody and a constant region of a human immunoglobulin). Chimeric antibodies are preferably used in order to, on the one hand, reduce the immunogenicity during application and, on the other hand increase the production yields; murine monoclonal antibodies, for example, produce higher yields from hybridoma cell lines and also cause higher immunogenicity in humans so that preference is given to using human/murine chimeric antibodies. Chimeric antibodies and methods for their preparation are known from the prior art (Cabilly et al., Proc. Natl. Sci. USA 81: 3273-3277 (1984); Morrison et al. Proc. Natl. Acad. Sci. USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci. USA 84:3439-3443 (1987); Sun et al.; Proc. Natl. Acad. Sci. USA 84:214218 (1987); Better et al., Science 240: 1041-1043 (1988) and Harlow and Lane, Antikörper: A Laboratory Manual, as cited above. These references are incorporated as part of the disclosure into the present invention.

An inventive antibody of this kind will be very particularly preferably directed against an epitope in the form of an extracellular section on an ee3 protein of the invention, in particular a protein according to FIGS. 13, 14, 15A, 15B, 15C, 16 or 18. Inventive antibodies, in all variations as disclosed previously, may be used for inhibiting inventive proteins of the ee3 family, for example in vitro for experimental studies, in situ, for example for labeling purposes, or else in vivo for therapeutic use by injecting them, for example, intravenously, subcutaneously, intraarterially or intramuscularly.

An anti-idiotypic antibody of the invention is an antibody which recognizes a determinant usually associated with the antigen-binding site of an antibody of the invention. An anti-idiotypic antibody may be prepared by immunizing an animal of the same species and of the same genetic type (e.g. a mouse strain) as starting point for a monoclonal antibody against which an anti-idiotypic antibody of the invention is directed. The immunized animal will recognize the idiotypic determinants of the immunizing antibody by producing an antibody directed against said idiotypic determinants (namely an anti-idiotypic antibody of the invention) (U.S. Pat. No. 4,699,880). An anti-idiotypic antibody of the invention may also be used as an immunogen in order to evoke an immune response in another animal and to lead to the production of an “anti-anti-idiotypic antibody” there. Said anti-anti-idiotypic antibody may, but need not, be identical, with respect to its epitope construction, to the original monoclonal antibody which caused the anti-idiotypic reaction. In this way it is possible, using antibodies directed against idiotypic determinants of a monoclonal antibody, to identify other clones expressing antibodies of identical specificity.

Monoclonal antibodies directed against proteins of the invention, analogs, fragments of derivatives of said proteins of the invention may be used for inducing binding of anti-idiotypic antibodies in corresponding animals such as, for example, the BALB/c mouse. Cells from the spleen of such an immunized mouse may be used for producing anti-idiotypic hybridoma cell lines which secrete anti-idiotypic monoclonal antibodies. Anti-idiotypic monoclonal antibodies may furthermore also be coupled to a support (KLH, keyhole limpet hemocyanin) and then be used for immunizing further BALB/c mice. The sera of these mice then contain anti-anti-idiotypic antibodies which have the binding properties of the original monoclonal antibodies and are specific for an epitope of the protein of the invention or of a fragment or derivative thereof. In this way, the anti-idiotypic monoclonal antibodies have their own idiotypic epitopes or “idiotopes” which are structurally similar to the epitope to be studied.

The term “antibodies” is intended to include both intact molecules and fragments thereof. Fragments which may be mentioned are any truncated or altered antibody fragments having one or two antigen-complementary binding sites, such as antibody moieties having a binding site corresponding to said antibodies and composed of light and heavy chains, such as Fv, Fab or F(ab′)₂ fragments or single strand fragments. Preference is given to truncated double strand fragments such as Fv, Fab or F(ab′)₂. Fab and F(ab′)₂ fragments lack an Fc fragment present, for example, in an intact antibody, so that they can be transported more rapidly in the blood stream and have comparatively less nonspecific tissue binding than intact antibodies. It is emphasized here that Fab and F(ab′)₂ fragments of antibodies of the invention, as well as these antibodies themselves, may be used in detecting (qualitatively) and quantifying proteins of the invention (where appropriate, also for detecting protein activity (e.g. specific phosphorylations) of the proteins of the invention), as a result of which methods for qualitative and quantitative determination and/or quantification of the protein activity of proteins of the invention are likewise a subject matter of the present invention.

Fragments of this kind are typically prepared by proteolytic cleavage by using enzymes such as, for example, papain (for preparing Fab fragments) or pepsin (for preparing F(ab′)₂ fragments), or said fragments are obtained by chemical oxidation or genetic manipulation of the antibody genes.

The present invention also relates to mixtures of antibodies for the purposes of the present invention. Besides said antibodies, it is also possible to use mixtures of antibodies for any methods or uses described according to the present invention. Purified fractions of monoclonal antibodies, polyclonal antibodies or mixtures of monoclonal antibodies are used as drugs and employed in the preparation of drugs for the treatment of cerebral ischemias (e.g. stroke), degenerative disorders, in particular neurodegenerative disorders, and neurological disorders such as epilepsy, for example.

Antibodies of the invention, including the fragments of these antibodies and/or mixtures thereof may be used for quantitative or qualitative detection of ee3 gene product of the invention, in particular proteins according to FIGS. 13, 14, 15A, 15B, 15C, 16 or 18 or fragments or derivatives thereof, in a sample or else for detecting of cells expressing and, where appropriate, secreting proteins of the invention. In this respect, the use of antibodies of the invention as diagnostics is disclosed. Thus, it is possible, for example, to determine via antibodies of the invention the amount of gene product of the invention and possibly the activity thereof (e.g. specific phosphorylations), for example of the proteins according to FIGS. 13, 14, 15A, 15B, 15C, 16 or 18. Detection may be achieved with the aid of immunofluorescence methods which are carried out fluorescently labeled antibodies in combination with light microscopy, flow cytometry or fluorimetric detection.

Inventive antibodies in accordance with the invention (this includes fragments of said antibodies or else mixtures of antibodies) are suitable for histological studies, for example in the course of immunofluorescence of immunoelectron microscopy, for in situ detection of a protein of the invention. In situ detection may be carried out by taking a histological sample from a patient and adding to such a sample labeled antibodies of the invention. The antibody (or a fragment of this antibody) is applied in a labeled form to the biological sample. In this way it is possible to determine not only the presence of protein of the invention in the sample but also the distribution of said protein of the invention in the tissue studied. The biological sample may be a biological fluid, a tissue extract, harvested cells such as, for example, immunocells or cardiomyocytes or hepatocytes, or generally cells which have been incubated in a tissue culture. Detection of the labeled antibody may be carried out using methods known in the prior art, depending on the type of labeling (e.g. by fluorescence methods). However, the biological sample may also be applied to a solid support such as, for example, nitrocellulose or another support material, so as to immobilize the cells, cell parts or soluble proteins. The support may then be washed once or several times with a suitable buffer, followed by treatment with a detectable labeled antibody according to the present invention. The solid support may then be washed a second time with the buffer in order to remove unbound antibodies. The amount of bound label on the solid support may then be determined using a conventional method.

Suitable supports are in particular glass, polystyrene, polypropylene, polyethylene, dextran, nylon amylases, natural or modified celluloses, polyacrylamides and magnetite. The support may either have limited solubility or be insoluble in order to meet the conditions in accordance with the present invention. The support material may come in any shape, for example in the shape of beads, or may be cylindrical or spherical, with the preferred support being polystyrene beads.

Detectable antibody labeling may be carried out in various ways. For example, the antibody may be bound to an enzyme which may ultimately be used in an immunoassay (EIA). Said enzyme may then later react with a corresponding substrate so as to produce a chemical compound which may be detected and, where appropriate, quantified in a manner familiar to the skilled worker, for example by spectrophotometry, fluorometry or other optical methods. The enzyme may be malate dehydrogenase, staphylococcus nuclease, delta-5-steroid isomerase, yeast alcohol dehydrogenase, alpha-glycerophosphate dehydrogenase, triosephosphate isomerase, horseradish peroxidase, alkaline phosphatase, asparaginase, glucose oxidase, beta-galactosidase, ribonuclease, urease, catalase, glucose 6-phosphate dehydrogenase, glucoamylase or acetyl choline esterase. Detection is then made possible via a chromogenic substrate specific for the enzyme used for labeling and may ultimately be carried out, for example, via visual comparison of the substrate converted by the enzyme reaction in comparison with control standards.

Furthermore, detection may be ensured using other immunoassays, for example radiolabeling of the antibodies or antibody fragments (i.e. a radioimmuno-assay (RIA; Laboratory Techniques and Biochemistry in Molecular Biology, Work, T. et al. North Holland Publishing Company, New York (1978). The radioisotope may be detected and quantified by using scintillation counters or by autoradiography.

Fluorescent compounds may likewise be used for labeling, for example compounds such as fluorescein isothiocyanate, rhodamine, phycoerythrin, phycocyanine, allophycocyanine, o-phthaldehyde and fluorescamine. Fluorescence-emitting metals, such as, for example, ¹⁵²E or other metals of the lanthanide group, may also be used. These metals are coupled to the antibody via chelating groups such as, for example diethylenetriaminepentaacetic acid (ETPA) or EDTA. The antibody of the invention may furthermore be coupled via a compound acting with the aid of chemiluminescence. The presence of the chemiluminescently labeled antibody is then detected via the luminescence produced in the course of a chemical reaction. Examples of such compounds are luminol, isoluminol, acridinium esters, imidazole, acridinium salt or oxalate esters. It is equally possible to use also bioluminescent compounds. Bioluminescence is a subtype of chemiluminescence, which is found in biological systems and in which a catalytic protein enhances the efficacy of the chemiluminescent reaction. The bioluminescent protein is again detected via luminescence, suitable examples of bioluminescent compounds being luciferin, luciferase and aequorin.

An antibody of the invention may also be employed for use in an immunometric assay, also known as “two-site” or “sandwich” assay. Typical immunometric assay systems include “forward” assays which are distinguished by inventive antibodies being bound to a solid phase system and by contacting in this way the antibody with the sample studied. In this way, the antigen is isolated from the sample by forming a binary solid phase antibody-antigen complex from the sample. After a suitable incubation period, the solid support is washed in order to remove the remaining residue of the liquid sample, including possibly unbound antigen, and then contacted with a solution containing an unknown quantity of labeled detection antibody. The labeled antibody here serves as a “reporter molecule”. After a second incubation period which allows the labeled antibody to associate with the antigen bound to the solid phase, the solid phase support is washed again in order to remove unreacted labeled antibodies.

An alternative assay form may also make use of a “sandwich” assay. In this case, a single incubation step may be sufficient if both the solid phase-bound antibody and the labeled antibody are applied simultaneously to the sample to be assayed. After the incubation has ended, the solid phase support is washed in order to remove residues of the liquid sample and of the non-associated labeled antibodies. The presence of labeled antibody on the solid phase support is determined in the same way as in the conventional “forward” sandwich assay. The “reverse” assay involves first adding step by step a solution of the labeled antibody to the liquid sample, followed by admixing unlabeled antibody bound to a solid phase support, after a soluble incubation period. After a second incubation step, the solid phase support is washed in a conventional manner in order to remove therefrom sample residues and unreacted labeled antibody. The labeled antibody which has reacted with the solid phase support is then determined as described above.

The present invention furthermore discloses methods for expressing the ee3 gene products of the invention, i.e. in particular polypeptides according to FIGS. 13, 14, 15A, 15B, 15C, 16 or 18, including any derivatives, analogs and fragments, host cells being transformed for this with an expression vector of the invention. This method for expressing gene products based on a DNA sequence of the invention does not serve to concentrate and purify the corresponding gene product but rather serves to influence the cellular metabolism by introducing the DNA sequences of the invention via expression of the corresponding gene product. In particular here is to the use of the host cells transformed with the aid of expression vectors as drugs or for preparing a drug, in particular for purposes of treating disorders, for example oncoses, neurological disorders, neurodegenerative disorders (e.g. multiple sclerosis, Parkinson's disease) cerebral ischemias (e.g. stroke). Host cells of the invention are generally provided for disorders based on dysregulation of apoptosis, necrosis, cell growth, cell division, cell differentiation or cell plasticity. The autologous or allogenic host cells transformed ex vivo in this way according to the invention may then be transplanted into patients.

Another aspect of the present invention comprises a method for isolating gene products having at least one partial sequence homologous to the amino acid sequences of the invention, in particular to the sequences with numbers 5, 6, 7 (including 7b and 7c), 8 and 11, at least over a partial sequence of at least 20, preferably at least 30, AA, which method involves transforming the host cells with an expression vector of the invention and then culturing said cells under suitable, expression-promoting conditions in such a way that the gene product can finally be purified from the culture. Depending on the expression system, the inventive gene product of the inventive DNA sequence may be isolated here from a culture medium or from cell extracts. It is readily apparent to the skilled worker that the particular isolation methods and the method for purifying the recombinant protein encoded by a DNA of the invention strongly depends on the type of host cell or else on the question, whether the protein is secreted into the medium. It is possible to use, for example, expression systems which cause the recombinant protein to be secreted from the host cells. In this case, the culture medium must be concentrated using commercially available protein concentration filters, for example Amicon or Millipore Pelicon. The concentration step may be followed by a purification step, for example a gel filtration step or purification with the aid of column chromatography methods. Alternatively, however, it is also possible to use an anion exchanger having a DEAE matrix.

The matrix used may be any materials known from protein purification, for example acrylamide or agarose or dextran or the like. It is, however, also possible to use a cation exchanger which then typically contains carboxymethyl groups. A polypeptide encoded by a DNA of the invention may be further purified using one or more HPLC steps. Particular use is made of the reversed phase method. Said steps serve to obtain an essentially homogeneous recombinant protein of a DNA sequence of the invention.

The gene product may also be isolated, using transformed yeast cells in addition to bacterial cell cultures. In this case, the translated protein can be secreted, thus simplifying protein purification. Secreted recombinant protein may be obtained from a yeast host cell by methods as disclosed in Urdal et al. (J. Chromato. 296:171 (1994)), which are part of the disclosure of the present invention.

Nucleic acid sequences of the invention, in particular DNA sequences of the invention, and/or gene products of the invention may be used as drugs or for the preparation of a drug. Said drugs may be administered on their own (e.g. buccally, intravenously, orally, parenterally, nasally, subcutaneously) or combined with other active compounds, excipients or drug-typical additives. The nucleic acid of the invention may be injected as naked nucleic acid, in particular intravenously, or else administered to the patient with the aid of vectors. These vectors may be plasmids as such or else viral vectors, in particular retroviral or adenoviral vectors, or else liposomes which may have naked DNA of the invention or a plasmid comprising DNA of the invention.

The use of sequences of the invention, in particular of the nucleotide or amino acid sequences 1 to 8 or their variants, and also of protein heteromers of the invention and also inventive reagents derived therefrom (oligonucleotides, antibodies, peptides) is thus suitable for preparing a drug for therapeutic purposes, i.e. for the treatment of disorders. Very particular preference is given here to the therapeutic use for the treatment or for preparing a drug for the treatment of disorders or pathophysiological states based on dysregulation of homeostasis of cell death and proliferation events.

In this context, the finding of the invention that EPO whose action can be attributed to the change in transcription behavior of cells induces transcriptional upregulation of receptors of the invention, e.g. ee3_{—}1, directly or indirectly also gains particular importance. Receptors of the invention thus mediate the action of EPO and are therefore also critically important for the disorders associated herewith. This means inter alia that receptors of the invention can selectively influence particular actions of EPO, for example a neuroprotective action (e.g. in neurodegenerative disorders), where, for example, activation of the transcription factor NF-kappaB is an important step in the neuroprotective action of EPO), or an increase in brain function (e.g. in dementia).

Corresponding studies on animal experiments allow subject matters of the invention to be functionally ascribed to models of neurological disorders such as cerebral ischemias, experimentally induced encephalomyelitis or subarachnoidal hemorrhages.

This is desirable for said partial actions, since administration of EPO would cause, in addition to the neuroprotective action, an increase in the hematocrit, which would partially conflict with said neuroprotective action, since the Theological properties of the blood would deteriorate, having an adverse effect on microcirculation, as has been shown in mice by overexpression of erythropoietin (Wiessner et al., 2001, J Cereb Blood Flow Metab, 21, 857-64).

Thus, the use of subject matters of the invention, for example of nucleotide sequences of the invention, oligo- or polypeptides, expression vectors, host cells or of surrogate ligands which are capable of attaching to any positions of receptors of the invention, which are relevant to regulation, is suitable in particular for preparing drugs for the treatment of neurological, in particular neurodegenerative, disorders.

The use of the invention can influence cell death processes, for example cascades leading to apoptosis, or processes leading to necrosis, in any cell types expressing inventive proteins of the ee3 family or a native variant thereof, in particular in neural cells, for example by modulating cell-cell interactions, in particular those involving G protein-coupled proteins.

According to the invention, the native proteins of the invention, in particular those having the sequences 5 to 8, are, as receptors, part of intracellular signal transduction pathways, typically as start of a signal cascade, dysregulation thereof being the cause of a multiplicity of disorders. In this respect, the abovementioned proteins of the invention can be found in particular as components in the following cellular processes and have cellular functions, for example in: signal transduction in general, with action on cell differentiation, cell division, growth, plasticity, regeneration, cell differentiation, proliferation or cell death. Accordingly, nonfunctionality of a protein of the invention, for example of ee3_{—}1 or ee3_{—}2, or nonfunctional expression or overexpression thereof can typically cause a pathophysiological condition which is accompanied by dysregulation of, for example, cell differentiation, cell growth, cell plasticity or cell regeneration. On the other hand, other mechanisms may also result in pathophysiological conditions, for example nonfunctionality or overfunctionality of the native ligand(s) of ee3 receptors of the invention. Depending on the molecular mechanism of the pathophysiological disorder, administration of a functional protein of the invention or at least higher expression of said protein or else inhibition of the cellularly overexpressed or expressed nonfunctional protein may be desired for therapeutic purposes. Very particular preference is given to the use of sequences of the invention, in particular sequences with numbers 1 to 11, in connection with their function in neuronal cell death, excitation and neurogeneses. These findings of the invention result in the use of sequences of the invention (nucleotide and amino acid sequences) and of corresponding derivatives (e.g. peptides, oligonucleotides or antibodies) for preparing a drug for treatment of oncoses and neurological disorders, in particular ischemic conditions (stroke), multiple sclerosis, neurodegenerative disorders such as, for example, Parkinson's disease, amyotrophic lateral sclerosis, heredodegenerative ataxias, neuropathies, Huntington's disease, epilepsies and Alzheimer's disease. In addition, owing to upregulation in the case of increased erythropoietin expression, the use of subject matters of the invention is suitable for any pathological processes in which EPO plays a (protective) part (e.g. stroke, and any forms of acute and chronic hypoxias).

According to the invention, cell-based HTS assays for functional receptor activation, measured by enzyme complementation, prove suitable in order to obtain further indications on the basis of molecular relationships. The assay is based on the general regulatory mechanism of GPCRs and measures the interaction between activated receptor and beta-arrestin. For this purpose inactive beta-galactosidase fragments complementing each other are fused to the C terminus of the receptor and to beta-arrestin. Activation of said receptor recruits beta-arrestin. This brings together the two halves of beta-galactosidase, resulting in a functioning beta-galactosidase enzyme capable of converting corresponding substrates which serve as the measured signal (ICAST system). It is possible in principle to carry out said assay with any enzymes that are capable of being expressed as fusion proteins of two halves complementing each other and carry out a substrate reaction recordable by common measurement methods.

The present invention further relates to the therapeutic application of sequences of the invention, namely the use of a nucleic acid sequence or protein sequence of the invention, in particular the nucleotide sequence or amino acid sequence numbered 1 to 4 or 5 to 8, or of a variant, as defined above, thereof, in particular of a fragment, for gene therapy in mammals, for example in humans, or else gene therapy methods of this kind. Gene therapy here includes any forms of therapy that either introduce sequences of the invention as claimed in any of claims 1 to 4 into the body or parts thereof, for example individual tissues, or influence expression of sequences of the invention. For this purpose, any modifications familiar to the skilled worker may be used in the course of gene therapy, for example oligonucleotides, e.g. antisense or hybrid RNA-DNA oligonucleotides, having any modifications and comprising sequences of the invention may be utilized. It is likewise possible to utilize viral constructs comprising any sequences of the invention (this includes any variants such as fragments, isoforms, alleles, derivatives). Corresponding naked DNA sequences of the invention are also suitable in gene therapy. Likewise it is possible to utilize nucleic acid pieces having enzymic activity (i.e. ribozymes) for gene therapy purposes.

Aside from therapeutic applications, diagnostic uses of nucleic acids or polypeptides of the invention, of protein heteromers of the invention and also of inventive reagents derived therefrom (oligonucleotides, antibodies, peptides) are also suitable, for example for diagnosing human disorders or genetic predispositions, for example also in the course of pregnancy tests. Said disorders or predispositions are in particular the disorders mentioned above in connection with therapeutic application, especially neurological or immunological disorders or oncoses. These diagnostic methods may be designed as in vivo, but typically ex vivo, methods. A typical ex vivo application of a diagnostic method of the invention will be useful for qualitative and/or quantitative detection of a nucleic acid of the invention in a biological sample. A method of this kind preferably comprises the following steps: (a) incubating a biological sample with a known amount of nucleic acid of the invention or a known amount of oligonucleotides suitable as primers for amplification of said nucleic acid of the invention, (b) detecting said nucleic acid of the invention by specific hybridization or PCR amplification, (c) comparing the amount of hybridizing nucleic acid or of nucleic acid obtained by PCR amplification with a quantity standard. Moreover, the invention relates to a method for qualitative and/or quantitative detection of a protein heteromer of the invention or of a protein of the invention in a biological sample, which method comprises the following steps: (a) incubating a biological sample with an antibody specifically directed against said protein heteromer or against the protein/polypeptide of the invention, (b) detecting the antibody/antigen complex, (c) comparing the amounts of antibody/antigen complex with a quantity standard. The standard is usually a biological sample taken from a healthy organism. It is possible here, in particular for diagnostic purposes, to utilize the property of a gene of the invention, for example of the ee3_{—}1 gene, that, after characteristic pathophysiological stimuli (stroke, cardiac arrest, oncose etc.), a change, for example an increase, in the cellular amount of mRNA of sequences of the invention takes place. In this manner, it is possible, according to the invention, to carry out a prognosis of diseases accompanied by alterations in the rate of expression of proteins of the invention (such as, for example, stroke), the assessment of successful therapies or the classification of a disease. Finally, methods of the invention may be used for monitoring the treatment of disorders indicated above.

Sequences of the invention may be used in methods for determining polymorphisms of said sequences, for example in humans. These determined polymorphisms of sequences of the invention are not only subject to the disclosure of the present invention but may also serve prognostic markers for diagnosis or for diagnosing a predisposition of disorders associated with a due to nonfunctional expression of sequences of the invention, expression of nonfunctional sequences of the invention and/or overexpression thereof. In addition, sequences of the invention allow research into human genetic diseases, that is both monogenic and polygenic disorders.

In addition to therapeutic and/or diagnostic use purposes in the field of human and/or veterinary medicine, the use of nucleic acids or polypeptides of the invention for scientific use is also considered. In particular, the sequences of the invention allow related sequences in unicellular or multicellular organisms to be identified in a manner known to the skilled worker, for example via cDNA libraries, or related sequences to be located in the human genome. The nucleotide sequences of the invention, in particular the sequences numbered 1 to 4 (including any variants), may thus be used for isolating, mapping and correlating with markers for human genetic diseases genes for mRNAs coding for said nucleic acids or functional equivalents, homologs or derivative thereof, for example in murine or other animal genomes and in the human genome, by homology screening using common methods. This procedure allows, for example, causal correlation of the chromosomal loci of sequences of the ee3 family in humans (chromosome 2 (2q14.2); X-chromosome (Xq28, LocusID: 84548); chromosome 5, chromosome 8, chromosome 3; chromosome 7) with particular phenotypically known genetic disorders, in particular also oncoses (e.g. hepatocellular carcinoma), thereby considerably simplifying the diagnosis of said disorders and making possible new therapeutic approaches. The same applies to the proteins of the invention.

It is thus possible to diagnose with the aid of nucleic acids of the invention in particular human genetic diseases, that is both monogenic and polygenic disorders, and, as a result, said nucleic acids are used as markers, giving rise to a diagnostic method of the invention for genetic disorders.

The invention discloses in particular an assay system for scientific application, which is based on amino acid and/or nucleotide sequences of the invention. In this connection, cDNA, genomic DNA, regulatory elements of the nucleic acid sequences of the invention and the polypeptide and also recombinant or nonrecombinant fragments thereof may be used for developing an assay system. Such an assay system of the invention is particularly suitable for measuring the activity of the promoter or of the protein in the presence of the test substance. Said assay system preferably comprises simple measurement methods (calorimetric, luminometric, fluorescence-based or radioactive methods) which allow rapid measurement of a multiplicity of test substances (Böhm, Klebe, Kubinyi, 1996, Wirkstoffdesign [Drug Design], Spektrum-Verlag, Heidelberg). The assay systems described allow chemical libraries to be screened for substances acting on proteins of the invention, in particular of sequences 5 to 8 (e.g. derivatives or fragments thereof) in an inhibitory or activating manner. The identification of such substances is the first step on the path of identifying novel medicaments acting specifically on ee3-associated signal transduction. This involves in particular providing assay systems which make use of the known properties of G protein-coupled proteins, for example the assay systems disclosed hereinbelow.

The biological activity of protein of the invention, in particular of proteins according to FIGS. 13, 14, 15A, 15B, 15C, 16 or 18, typically the biological activity associated with apoptosis, proliferation, regeneration, cell growth, can also be inhibited, as is desired, for example, for stroke, septic shock, GvHD (graft versus host disease), degenerative disorders, in particular neurodegenerative disorders, acute hepatitis or other indications disclosed herein, in that to introduce oligonucleotides of typically at least 10 nucleotides in length, which code for (a portion of) an antisense strand of the native sequences of the proteins having the sequence numbers 5, 6, 7A, 7B, 7C, 8 and, respectively, 11, into the affected cells by using methods familiar to the skilled worker. This results in translation of the native mRNA of proteins of the invention of the ee3 family, for example ee3_{—}1 or ee3_{—}2, being blocked in the appropriately transformed cells, resulting preferably in an increase in the viability of the transfected cell or in a modulating effect on cell growth, cell plasticity, cell proliferation. In this case too, the above-described method may be used with the aid of recombinant viruses.

It is also possible to treat possibly pathologically increased cell apoptosis, cell proliferation in disorders based on a corresponding dysregulation of sequences of the invention (for example in the aforementioned indications) by using ribozyme methods. To this end, ribozymes capable of cutting a target mRNA are used. In this case, the present invention therefore discloses and relates to ribozymes capable of cleaving native ee3 mRNA, for example of ee3_{—}1 or ee3_{—}2. Ribozymes of the invention must be able to interact with the target mRNA of the invention, for example via base pairing, and subsequently cleave said mRNA in order to block translation of ee3_{—}1 or ee3_{—}2, for example. The ribozymes of the invention are introduced via suitable vectors into the target cells (in particular plasmids, modified animal viruses, in particular retroviruses), said vectors having, in addition to other sequences, where appropriate, a cDNA sequence for a ribozyme of the invention).

Modulation of the biological function of gene products of the invention, in particular of the gene products according to FIGS. 13, 14, 15A, 15B, 15C, 16 or 18, typically modulation of the function of gene products of the invention in apoptotic or necrotic, proliferative or growth-indicating signal transduction is, in addition to the abovementioned possibilities therefor, also possible with the aid of another subject matter of the present invention. A chemical compound of the invention will modulate, typically inhibit or else activate in particular the intracellular function of the inventive proteins of the ee3 family or influence the biological function at the level of the underlying DNA sequences of the invention, for example by binding to the DNA (e.g. the promoter region) or by binding to any of the transcription factors controlling a gene of the invention. Compounds of the invention will typically bind specifically to a protein of the invention, in particular to a protein having any of the amino acid sequences 1 to 4, or to a nucleic acid sequence of the invention, in particular to a nucleic acid sequence having any of the sequence reference numbers 5 to 8, and thereby cause a pharmacological, in particular neuroprotective or immunomodulating or anti-apoptotic or anti-proliferative action.

The invention therefore discloses chemical compounds, preferably an organochemical compound, having a molecular weight of <5000, in particular <3000, especially less than <1500, which is typically physiologically well tolerated and preferably capable of passing through the blood-brain barrier. Where appropriate, said compound will be part of a composition containing at least one further active compound and also preferably auxiliaries and/or additives and will be able to be used as a drug. Particular preference will be given to the organic molecule if the binding constant for binding to a protein of the invention, in particular to the C-terminal, cytosolic domain or to the extracellular domain of a protein of the invention, is at least 10⁷ mol⁻¹. The compound of the invention will preferably be designed so as to be able to pass through the cell membrane, either by way of diffusion or via (intra)membrane transport proteins, where appropriate after appropriate modification, for example with an attached AA sequence. Further preference is given to those compounds which inhibit or enhance the interaction of inventive proteins of the ee3 family with binding partners, in particular for transduction of an apoptotic or necrotic, proliferative or growth-indicating or regenerative signal. Compounds of this kind occupy in particular positions on the surface of proteins of the invention or cause a local conformation change in the proteins of the invention, thereby preventing binding of a native binding partner to a protein of the invention.

It is possible to find, via structural analyses of a protein of the invention, specifically compounds of the invention which have a specific binding affinity (Rationales Drug Design (Böhm, Klebe, Kubinyi, 1996, Wirkstoffdesign, Spektrum-Verlag, Heidelberg)). Here, the structure or a partial structure, derivative, allele, isoform or a part thereof of any of the proteins of the invention, in particular of any of the proteins having the sequences 5 to 8, is determined via NMR or X-ray crystallography methods (after appropriate crystallization, for example by the “hanging drop” method) or, if such a high resolution structure is not available, a structural model of a protein of the invention is produced with the aid of structure prediction algorithms, for example also with the aid of homologous proteins whose structure has already been elucidated (e.g. rhodopsin), and said structure or structural model is utilized in order to identify, with the aid of molecular modeling programs, compounds which may act as agonists or antagonists and which can be predicted to have high affinity to the protein of the invention. It is possible, where appropriate, for the methods defined above also to be combined with one another for the structural elucidation. Suitable force fields are employed to simulate the affinity of a compound potentially having affinity to a substructure of interest of interest of a protein of the invention, for example the active site, a binding cavity or a hinge region. These substances are then synthesized and tested in suitable test methods for their binding capacity and their therapeutic utilizability. Such in silico methods for identifying potential active compounds which display their action by binding to ee3 proteins of the invention are likewise a subject matter of the present invention.

In another preferred embodiment of the present invention, the compound of the invention is an antibody, preferably an antibody directed against an inventive protein of the ee3 family, for example ee3_{—}1, ee3_{—}2 or ee3_{—}5, or else an antibody directed against the underlying mRNA, which antibody is introduced ex vivo into retransplanted host cells or by means of in vivo gene therapy methods into host cells and which, as “intrabody”, is not secreted there but can display its action intracellularly. Such intrabodies of the invention can protect the cells against a misdirected apoptotic reaction, for example by overexpressing a protein of the invention. Such a procedure will be suitable typically for cells of those tissues which exhibit a pathophysiologically excessive apoptotic behavior in the patient, i.e., for example, pancreatic cells, keratinocytes, connective tissue cells, immuno-cells, neurons or muscle cells. In addition to the antibodies or intrabodies, cells genetically modified in this way with intrabodies of the invention are also part of the present invention.

A compound of the invention, having the function of blocking but also, where appropriate, of activating the biological function of native ee3 protein of the invention, for example of sequences numbered 5, 6, 7A, 7B, 7C, 8 and 11, or of corresponding native alleles or native splice variants, for example the apoptotic function, may be used as a drug. Compounds included here are any aforementioned variants, i.e., for example, organochemical compounds, antibodies, anti-sense oligonucleotides, ribozymes. A compound of the invention is particularly suitable (for preparing a drug) for the treatment of disorders, in particular for neurological, immunological or proliferative disorders. Thus it is possible for an inventive inhibitor (for example an antibody (in particular an intrabody) with inhibitory action, a ribozyme, antisense RNA, dominant-negative mutants or any of the aforementioned, where appropriate inhibitory, organochemical compounds, preferably a compound with high affinity, for example obtainable from any of the aforementioned methods) of the cellular function of a native protein of the invention, in particular of a protein having the sequences 5 to 8, or its native variants, i.e., for example, of the apoptotic response, to be used as a drug and very particularly for the treatment of the following disorders or for preparing a drug for the treatment of the following disorders: diseases in which chronic or acute states of hypoxia may occur or are involved, for example myocardial infarct, heart failure, cardiomyopathies, myocarditis, pericarditis, perimyocarditis, coronary heart disease, congenital heart defects with right-left shunt, tetralogy/pentalogy of Fallot, Eisenmenger syndrome, shock, hypoperfusions of extremities, arterial occlusive disease (AOD), peripheral AOD (pAOD), carotid stenosis, renal artery stenosis, small vessel disease, intracerebral bleeding, cerebral vein and sinus thromboses, vascular malformations, subarachnoidal hemorrhages, vascular dementia, Biswanger's disease, subcortical arteriosclerotic encephalopathy, multiple cortical infarcts during embolisms, vasculitis, diabetic retinopathy, consecutive symptoms of anemias of different causes (e.g. aplastic anemias, myelodysplastic syndrome, polycythemia vera, megaloblastic anemias, iron deficiency anemias, renal anemias, spherocytosis, hemolytic anemias, thalassemias, hemoglobinopathies, glucose 6-phosphate dehydrogenase deficiency, transfusion incidents, rhesus incompatibilities, malaria, heart valve replacement, hemorrhagic anemias, hypersplenism syndrome), lung fibroses, emphysema, lung edema: ARDS, IRDS, recurring pulmonary embolisms.

Oncoses (e.g. colon carcinoma, mammacarcinoma, prostate carcinoma, lung carcinom), disorders of the immune system (e.g. autoimmune disorders, in particular diabetes, psoriasis, immunodeficiencies, multiple sclerosis, rheumatoid arthritis or atopies, asthma), viral infectious diseases (e.g. HIV, hepatitis B or hepatitis C infections, bacterial infections (e.g. streptococcal or staphylococcal infections), degenerative disorders, in particular neurodegenerative disorders, for example muscular dystrophies, GvHD (e.g. liver, kidney or heart), or else neurological disorders (in particular, but not exclusively: stroke, multiple sclerosis, Parkinson's disease, subarachnoidal hemorrhages, amyotrophic lateral sclerosis, heredodegenerative ataxias, Huntington's disease, neuropathies, epilepsies, brain injuries, Alzheimer's disease); muscle relaxants (e.g. for anesthetizing), endocrinological disorders (e.g. osteoporosis or thyroid malfunctions) and dermatological disorders (psoriasis, neurodermititis); control of chronic or acute states of pain, genetic diseases, also disorders in the psychological field (e.g. schizophrenia or depressions), wound healing, support of sexual function, cardiovascular disorders (e.g. ischemic infarct, heart failure, arrythmias, hypertension), increase in cerebral function.

All of the aforementioned fields of indication also apply to the use of gene products of the invention or of DNA sequences of the invention for preparing a drug.

The aforementioned substances of the invention may also be part of a pharmaceutical composition which may contain further pharmaceutical carriers, excipients and/or additives, in order, for example, to stabilize such compositions for therapeutical administration or to improve biological availability and/or pharmacodynamics.

The present invention further relates to methods (screening methods) for identifying pharmaceutically active compounds, in particular those having inhibitory properties, with regard to triggering or transducing signals associated with physiological responses caused by sequences of the invention. Such pharmaceutically active substances may block receptors of the invention at their extracellular terminus, their TM domains (here, for example, impair di- or multimerization thereof), and also, intracellularly, signal transduction, for example block or activate the interaction between ee3 proteins and intracellular signal proteins, in particular influence (activate or inhibit) interaction with the intracellular proteins ranbpm and/or MAP1a/MAP1b.

Methods of the invention provide for (a) cells to be transfected with an expression vector as claimed in claim 5, in particular an expression vector coding for a polypeptide of the invention, for example for a polypeptide having the sequence numbers 5, 6, 7A, 7B, 7C, 8 or 11, and, where appropriate, with at least one expression vector coding for at least one reporter gene, and (b) a parameter suitable for observing the function mediated by proteins of the invention, for example signal transduction for regenerative or proliferative processes, said parameter being in particular caspase-3 activation, to be measured for the host cell system obtained according to (a) after addition of a test compound, in comparison with a control without addition of a test compound. To this end, preferably multiple parallel experiments with increasing concentrations of said test substance are set up according to the method of the invention in order to be able to determine the ID₅₀ of said test substance in the case of a pharmaceutical activity, for example the apoptosis-inhibiting action, of said test substance.

The knowledge of the primary sequence of ee3 proteins may be utilized in order to prepare recombinant constructs which make use of the properties of already characterized GPCRs known according to the prior art. Thus it is possible, for example, to replace particular sequence regions of ee3 proteins of the invention with particular sequence regions of a known, well characterized GPCR. The resulting construct may be employed for identifying, by means of known ligands or agonists, G protein coupling and the second messenger systems utilized or to utilize known G protein coupling for finding ligands, agonists or antagonists. Chimeric receptors of the invention, for example for the aforementioned uses, may be prepared, for example, according to a method as described by Kobilka et al. (and included in the present invention) (Kobilka B K, Kobilka T S, Daniel K, Regan J W, Caron M G, Lefkowitz R J (1988) Chimeric alpha 2-, beta 2-adrenergic receptors: delineation of domains involved in effector coupling and ligand binding specificity. Science 240:1310-1316).

It is furthermore possible to use according to the invention constitutively active receptor mutants of the ee3 family for characterizing the effect of said receptors on signal transduction pathways and for screening for ligands. Receptors of the invention as representatives of the 7TM-protein class may be mutated in a particular manner in order to evoke changes in the physiological and pharmacological behavior of said receptors. This may also be utilized, for example, for identifying intracellular signal pathways when the natural ligand or an agonist is unknown. Particularly suitable for causing such changes are mutations in the DRI consensus sequence of ee3 proteins; for example, mutation of R in the DRY sequence (Scheer A, Costa T, Fanelli F, De Benedetti P G, Mhaouty-Kodja S, Abuin L, Nenniger-Tosato M, Cotecchia S (2000) Mutational analysis of the highly conserved arginine within the Glu/Asp-Arg-Tyr motif of the alpha(1b)-adrenergic receptor: effects on receptor isomerization and activation. Mol Pharmacol 57:219-231)) to Lys, His, Glu, Asp, Ala, Asn and Ile causes, in the case of mutation to Lys, a strong increase in constitutive activation. A mutation to His or Asp result in a smaller increase in constitutive activation. Interestingly, mutation to Arg increases agonist affinity so that those mutants are also of interest for HTS screens.

Similarly, a conserved Arg in the third TM domain is a possible site of mutation (Ballesteros J, Kitanovic S, Guarnieri F, Davies P, Fromme B J, Konvicka K, Chi L, Millar R P, Davidson J S, Weinstein H, Sealfon S C (1998) Functional microdomains in G-protein-coupled receptors. The conserved arginine-cage motif in the gonadotropin-releasing hormone receptor. J Biol Chem 273:10445-10453).

Alternatively, methods based on the use of immobilized functional receptors of the invention may be used for identifying endogenous or surrogate ligands. In this case, inventive receptors of the ee3 family are expressed as fusion proteins with GST, the Flag tag or the TAP tag, as disclosed according to the invention. The corresponding cells are either processed according to common methods to give membranes or used directly for solubilization. Suitable detergents, for example dodecylmaltoside, digitonin, cholate or mixtures of detergents, are used to solubilize the receptors which are then bound to the corresponding affinity matrices such as GST Sepharose, anti-Flag M2 agarose or IgG Sepharose etc. The matrices are washed, then incubated with tissue extracts or cell supernatants and again washed. If the extract contains an active ligand, for example a peptide, then said ligand binds to the immobilized receptor and can be identified, after elution, by analytical methods, for example by means of mass spectrometry.

According to the invention, “internalization assays” represent another procedure for being able to identify natural or, in particular, surrogate ligands for receptors of the invention, for example ee3_{—}1. Here, use is likewise made of the different properties of a protein of the GPCR class. It is possible, for example, to use the internalization behavior of proteins of the GPCR class. This is to be understood as a regulatory mechanism after activation of the receptor. A screening method based on this behavior has the advantage of not needing a more detailed knowledge of the physiology of the particular receptor. In particular, no knowledge of the coupling G proteins and of the signal transduction pathways utilized is needed.

An assay of this kind is described, for example, by Lenkei et al. (2000, J Histochem Cytochem, 48, 1553-64) and may be used analogously according to the invention for the receptors of the invention. To this end, according to the invention, first a C-terminal fusion construct of protein of the invention with EGFP is prepared. This is followed by preparing stable CHO cells according to standard methods. Stable clones are selected with the aid of an FACS sorter for EGFP fluorescence. The final selection was carried out with the aid of fluorescence-microscopic assessment of surface expression. The cells are then incubated with HPLC fractions of tissue extracts, and internalization is determined with the aid of a confocal microscope. The evaluation is carried out with the aid of morphometric software (NIH Image), following the principle of the distance of fluorescent signals from the cell center. A frequency/distance distribution produced good discrimination for said internalization.

To find unknown ligands, successive fractionations are carried out to isolate the corresponding peptide to purity and then to identify said peptide by sequencing or MALDI-TOF, for example. Another application of, in principle, the same method is described by Ghosh et al. (2000, Biotechniques 29, 170-5; Conway et al., 1999, J Biomol Screen, 4, 75-86), both applications being incorporated in their entirety in the present disclosure.

However, it is also possible to use a functional Ca assay for identifying ligands and/or, where appropriate, also for characterizing the receptor of the invention. According to the invention, use is made here of the fact that a multiplicity of 7TM receptors (receptors with 7 transmembrane domains) produced in HEK293 cells, in CHO cells or in other cells result, via coupling to G proteins of the Gq class, in activation of PLC and mobilization of intracellular Ca. If certain receptors of the invention were not to couple to G proteins of the Gq class, then said receptors can be forced to give signal transduction via PLC, i.e. to produce Ca release, by co-expressing chimeric G proteins or the G proteins G15 or G16 which couple relatively unspecifically to receptors. The inventive receptors of the ee3 family are typically expressed in HEK293 and in CHO cells both stably and transiently alone and together with the chimeric G protein Gqi5 and alternatively with the G protein Gq15. The cells are then preferably loaded with a membrane-permeable Ca-binding fluorescent dye, for example Fura-2 or Fluo-3 or -4, and, after washing of the cells, treated with various test substances, measuring at the same time Ca release, for example using an FLIPR instrument from Molecular Devices. Finally, test substances giving a positive signal are preferably tested in control cells (transfected only with the vector) and, if the signal is found to be specific, pharmcologically characterized, i.e. by means of dose-response curves.

Alternatively, however, the Ca response caused by a ligand may also be measured using other Ca detectors, for example by AequoScreen from Euroscreen (Brussels, Belgium; see, for example, http://www.pharmaceutical-technology.com/contractors/compound_man/euroscreen/). This involves using cells which express the gene of the protein apoaequorin. Aequorin is produced after loading the cells with coelentrazine which binds to apoaequorin. If a ligand causes Ca to be released, said Ca activates aequorin to oxidize coelenterazine, thereby emitting light. The intensity of light emission is proportional to the increase in intracellular Ca concentration and thus a measure for the activity of the ligand found (taking into account the corresponding controls).

Antagonists are identified according to the invention by stimulating the receptors with a known agonist in the presence of sufficiently high concentrations of a large variety of ligands. An altered signal with respect to the control (only agonist, without another ligand), for example a lower Ca signal, indicates a competitive antagonist.

It is furthermore possible for cAMP assays to be used for characterizing the inventive receptors of the ee3 family and for identifying ligands. The background of this approach of the invention for pharmacological characterization of ee3 receptors and suitable for identifying ligands (agonists or antagonists) is the property of receptors of the class of GPCRs, i.e., for example, of proteins of the invention, to be able to act on adenylate cyclases either in a stimulating or inhibiting way, usually by activating “stimulating Gs” or “inhibiting Gi” proteins. Depending on the action of test substances, for example in a high throughput screening, it is possible to study via direct or indirect measurements the change in the cellular cAMP level associated therewith. This involves expressing the receptor genes stably or transiently in mammalian cells (see exemplary embodiment 2). In the case of GPCRs which activate adenylate cyclases, thereby increasing the cellular cAMP level, a potential agonist among the test substances is identified by way of an increased cAMP concentration compared to control cells. Antagonists among the test substances, for example in an HTS approach, are identified by way of their blocking the increase in cAMP concentration caused by an agonist. In the case of Gi-coupled ee3 receptors of the invention, the assay involves stimulating adenylate cyclase either directly with forskolin or by activating a Gs-coupled receptor, thereby increasing the cAMP level. An agonist of the Gi-coupled receptor inhibits this increase. A number of commercially available assays such as, for example, the cAMP^{[3H]} assay system from Amersham, which are based, for example, on the principle of competitive displacement of endogenously produced cAMP by added radiolabeled (tritium) cAMP, may be used for direct cAMP measurements. Indirect cAMP measurements are usually carried out by way of reporter assays. For this purpose, the receptors are expressed in cell lines containing reporter systems, for example the CRE-luciferase system. cAMP activates expression of luciferase whose activity is measured by converting corresponding substrates and luminometric measurement of the products. Reporter assays are very particularly suitable for mass screening methods.

Finally, it is also possible according to the invention to use, in addition to the above-described assays, the following assay systems for characterizing second-messenger systems of receptors of the invention and/or for identifying ligands of ee3 receptors of the invention, according to the invention in particular for determining the adenylate cyclase activity in cells or membranes according to Salomon (Salomon et al., (1979). Adv. Cyclic Nucleotide Res. 10, 35-55), for determining the inositol 3-phosphate concentration or for measuring a change in arachidonic acid release. For example, it is possible to overexpress ee3_{—}1 in common cell lines and, after activation by tissue extracts, to determine the activity of the second-messenger systems indicated above. Individually, assays for second-messenger systems of the GPCR class are well known to the skilled worker and, in individual cases, can be found in the literature, for example Signal Transduction: A practical approach, G. Milligan, Ed. Oxford University Press, Oxford, England. Further reporter assays for screening include MAP kinase/luciferase and NFAT luciferase systems.

Based on the finding of the invention that ee3 receptor signal transduction also takes place via MAP kinase signal transduction pathways, can also be used for developing screening assays for searching for ligands or identifying inhibitors, for example via an NF-kB reporter systems or luciferase systems.

As mentioned above, activation of second messenger also serves to identify ligands, agonists or antagonists binding to receptors of the invention and being able in this way to display their agonistic and/or antagonistic action on certain cellular processes. For example, microphysiometers may be used for identifying ligands, agonists or antagonists. Signals caused by ligand binding to a receptor of the ee3 family represent energy-consuming processes. Therefore, processes of this kind are always accompanied by slight metabolic changes, inter alia a slight pH shift. Said changes may be recorded extracellularly by a microphysiometer (Cytosensor, Molecular Devices), for example.

After identification of ligands, agonists or antagonists having a potential of binding to inventive proteins of the ee3 receptor family, they may be characterized in more detail according to the invention by carrying out ligand binding assays. Ligand binding assays enable the pharmacology of a receptor, i.e. the affinity of a large variety of ligands for said receptor, to be measured directly. For binding studies, typically a chemically pure ligand identified by any of the aforementioned methods or known in some other way is here radiolabeled with a high specific activity (30-2000 Ci/mmol) in such a way that the radiolabel does not reduce the activity of said ligand with respect to the receptor. The assay conditions are optimized both for the use of cells expressing said receptor and of membranes prepared therefrom with respect to buffer composition, salt, modulators such as, for example, nucleotides or stabilizers such as, for example, glycerol in such a way that a usable signal-to-background ratio is measured. Specific receptor binding is defined for these binding assays as the difference of total radioactivity associated with receptor preparation (cells or membranes), i.e. measured in the presence of only one specific, namely the radioligand, and the radioactivity measured in the presence of both the radioligand and an excess of non-radiolabeled ligand. The unlabeled ligand here competitively displaces the radioligand. If possible, at least two chemically different competing ligands are used in order to determine nonspecific binding. Optimal specific binding is one which is at least 50% of total binding. The binding assay is carried out either inhomogeneously as filtration assay or homogeneously as scintillation proximity assay.

In the first case, the receptor-containing preparation (cells or membranes) is incubated with the ligands in a suitable buffer solution, until binding equilibrium has formed, typically at RT for 1 h and at 4° C. overnight, and then filtered off via suitable filters, for example glass fiber filters from Whatman or Schleicher & Schuell which have been pretreated, where appropriate, for example with polyethylenimine, in order to separate unbound from bound radioligand. The filters are washed and then dried or, in a wet state, treated with appropriate scintillator and, after incubation which may be required, the radioactivity obtained is measured in a scintillation counter. The scintillation proximity assay involves incubating suitable scintillation beads, for example WGA beads, with the ligands and receptor-containing membranes in a suitable buffer solution, until binding equilibrium has formed, and radioactivity is then measured in a suitable scintillation counter. Both binding assays may be performed in the HTS format.

Solubilized or purified receptors are measured using the scintillation proximity assay or common inhomogeneous assays such as the filtration assay after PEG precipitation, the adsorption assay or the gel filtration assay (Hulme E, Birdsall N (1986) Distinctions in acetylcholine receptor activity. Nature 323:396-397).

It is also possible to use a fluorescent ligand, for example a ligand covalently bound to a fluorescent dye such as BODIPY, rather than a radioligand. Binding of the fluorescent ligand to the receptor is measured by means of fluorescence polarization. The method is suitable both for primary screenings in HTS format and in secondary assays.

The present invention furthermore discloses in a preferred embodiment high throughput screening assays (HTS) for identifying ligands (agonists or antagonists), in particular inhibitors of ee3 sequences of the invention. Very particular preference is given here to using (all known) components of the MAP signal transduction pathway within the scope of the method of the invention for identifying inhibitors, in particular for identifying small organic compounds. Suitable systems are preferably those comprising the scintillation proximity assay (SPA) (Amersham Life Science, MAP kinase. SPA (see McDonald et al., 1999, Anal Biochem, 268, 318-29)). Said application is incorporated in its entirety in the disclose of the present invention. Here, the MAP cascade is reconstituted in vitro, prepared with the individual components being GST fusion proteins (E. coli-expressed) or, in the case of cRAF1, prepared using the baculovirus system. The first element of the cascade (MAP-KKK) must be activated permanently and evenly here in order to be able to assay inhibitors in a reliable manner. This is typically achieved by coexpressing src in the baculovirus system. This ensures a ras-like activation of cRaf. After transfection of nucleotide sequences of the invention, a modulation of the cascade is caused, which modulation is used in order to be able to measure in an HTS an influence on said modulation by adding substances to be assayed.

After identification of selective substances with high affinity by the aforementioned methods of the invention, said substances are assayed for their use as medicaments for epilepsy, stroke and other neurological, immunological or proliferative disorders (oncoses). In addition, it is possible to determine the binding sites of the identified and pharmacologically active substances to the ee3 gene products of the invention, in particular the sequences with numbers 5, 6, 7A, 7B, 7C, 8 or 11, with the aid of the yeast-two hybrid system or other assays, i.e. to narrow down the amino acids responsible for the interaction, for example also for the interaction between native proteins. In a next step it is possible to identify substances with high affinity (surrogate ligands) which especially have to the previously identified amino acids responsible for binding of the native interaction partners (structural regions) by the screening methods described in the present patent application. In this way, it is also possible to find substances which can be used to influence, in particular inhibit, the interaction between polypeptides of the invention and possible native intracellular interaction partners thereof. This discloses according to the invention a method for finding substances with specific binding affinity for the protein of the invention. Particular reference is made in this connection to methods as described in Klein et al. (1998, Nat Biotechnol, 16, 1334-7). The known properties of a protein of the invention belonging to the class of the G protein-coupled receptors (coupling to G proteins, signal transduction) may moreover be utilized in order to identify inhibitors in accordance with the invention.

Owing to the pharmacological importance of inventive genes or inventive gene products of the ee3 family, in particular those in the ee3_{—}1 and ee3_{—}2 sequences, and/or their native variants for numerous disorders, for example in neurodegenerative, proliferative, i.e. in particular neoplastic, disorders (oncoses, for example solid tumors (sarcomas (sarcomas of the skin (Kaposi sarcoma), blastomas, carcinomas of the liver, of the intestine, of the pancreas, of the stomach or of the lung) or tumors of the hematopoietic system, very particularly lymphomas or leukemias), or hypoapoptotic or hyperapoptotic disorders, pharmaceutically active substances identified according to the method of the invention have a broad spectrum of applications. In addition to the inhibition of an interaction with one or more other molecules, for example with protein kinases downstream in the signal transduction pathway, or adaptors, it is in particular also possible for influencing of transcription or of the amount of transcript of proteins of the invention in the cell to be the cause of pharmaceutical activity. An example which should be mentioned is fast upregulation of transcripts of DNA sequences of the invention after pathological processes being suppressed by compounds of the invention, in particular in the case of very rapid regulation thereof by transcriptional activation. A preferred target for a pharmaceutically active compound is therefore the regulation of transcription, for example by way of said substances specifically binding to a regulatory region (e.g. promoter or enhancer sequences) of a gene product of the invention, binding to one or more transcription factors of a gene product of the invention (resulting in an activation or inhibition of said transcription factor) or regulation of expression (transcription or translation) of such a transcription factor itself.

Aside from transcriptional regulation, i.e. regulating the amount of mRNA of a gene of the invention in the cell, a pharmaceutically active compound of the invention may also intervene in other cellular control processes which may influence, for example, the rate of expression of a protein of the invention (e.g. translation, splice processes, native derivatization of gene product of the invention, e.g. phosphorylation, or regulation of degradation of gene product of the invention.

The present invention further relates to methods for identifying cellular interaction partners of polypeptides of the invention from the ee3 family, i.e. in particular of proteins ee3_{—}1, ee3_{—}2 or ee3_{—}5 and/or their native variants (isoforms, alleles, splice forms, fragments). In this way it is possible for proteins to be identified as interaction partners which have specific binding affinities for the protein of the invention or for identifying nucleic acids coding for proteins which have specific binding affinities for the protein of the invention. Examples of cellular interaction partners of proteins of the ee3 proteins class of the invention may be other GPCRs or ion channels.

A method of the invention of this kind or the use of polypeptides of the invention, nucleic acid sequences of the invention and/or nucleic acid constructs of the invention for carrying out such methods is preferably carried out with the aid of a yeast two-hybrid screening (y2h screening) alone or in combination with other biochemical methods (Fields and Song, 1989, Nature, 340, 245-6). Screenings of this kind can also be found in Van Aelst et al. (1993, Proc. Natl. Acad. Sci. USA, 90, 6213-7) and Vojtek et al. (1993, Cell, 74, 205-14). Typically, it is also possible to use mammalian systems rather than yeast systems for carrying out a method of the invention, for example as described in Luo et al. (1997, Biotechniques, 22, 350-2). The corresponding aforementioned experimental approaches here make use of typical properties of the class of GPCR proteins, for example signal transduction, e.g. via G proteins, i.e., for example, also the known intracellular interaction partners.

For y2h screening, the open reading frame of sequences of the invention, in particular of sequences with numbers 1 to 4, or of a native variant, very particularly preferably intracellular regions of sequences of the invention, for example ee3-1 or ee3-2, are cloned for example into a “bait vector” in frame with the GAL4 binding domain (e.g. pGBT10 or pGBKT7 from Clontech). This can be used preferably to screen a “prey library” in a yeast strain for interacting proteins, following a familiar protocol. In addition, y2h systems may also be used to carry out “mapping experiments” in order to identify specific interaction domains.

Equally preferred are also two-hybrid systems utilizing other fusion partners or other cell systems, for example the BacterioMatchsystem from Stratagene or the CytoTrapsystem from Stratagene. As an alternative to the y2h methods, it is also possible according to the invention to use corresponding systems of mammalian cells, as described, for example, in Luo et al. (1997, Biotechniques, 22, 350-2) as part of the present disclosure.

It is also possible according to the invention to isolate interaction partners via co-immunoprecipitations from cells transfected with expression vectors of the invention in order to purify proteins binding thereto and subsequently to identify the corresponding genes via protein sequencing methods (e.g. MALDI-TOF, ESI-tandem-MALDI).

The present invention therefore further relates to the use of the yeast two-hybrid system or of corresponding methods known in the prior art or other biochemical methods for identifying interaction domains of ee3 proteins of the invention and/or of native variants of the latter and to the use of said interaction domains (fragments of the native sequences) for pharmacotherapeutic intervention.

Further methods of the invention for identifying endogenous or surrogate ligands, i.e. non-native compounds with properties of binding to the inventive receptors of the ee3 family, may be carried out with the aid of assays containing the following starting material: (a) a very wide variety of tissue extracts and cell culture supernatants of a large variety of cells which may also be pretreated with substances such as erythropoietin may be used. The extracts are then fractionated and the individual fractions in turn are used in the assay until the ligand is isolated. (b) A commercially obtained substance bank is used, for example LOPAC from Sigma, which contains potential ligands for orphan receptors, in particular (neuro)transmitters, bioactive peptides, hormones, chemokines and other naturally occurring substances which could bind to 7TM receptors according to the prior art and which therefore could also have the ability to bind to the inventive receptors of the ee3 family. (c) A combinatorial peptide library is used. Or (d): a commercially obtainable substance library whose composition may differ greatly is used.

Upregulation, for example, of ee3_{—}1 by EPO indicates that, for example, ee3_{—}1 is associated with the survival of cells, since EPO has neuroprotective actions. The polypeptides of the invention, in particular native forms or else non-native, artificially generated variants whose biological function is to be studied, may therefore be used according to the invention in an apoptosis assay or in a method for studying the function and/or efficacy of polypeptides of the invention in inducing, transducing or inhibiting cell death signals or other cell physiological processes. The involvement of inventive proteins of the ee3 family or of aforementioned inventive variants in, for example, apoptotic cascades may be studied by transfecting expression constructs containing ee3 sequences of the invention, in particular sequences with numbers 1 to 4, or variants into eukaryotic cells (as a result of which the use thereof for studies of this kind is also disclosed), and being able to study thereafter the induction of apoptosis. This may be effected, for example, by staining with annexin (Roche Diagnostics), by antibodies recognizing the active form of caspase-3 (New England Biolabs) or by ELISAs recognizing DNA-histone fragments (cell-death elisa, Roche Diagnostics). Said induction of apoptosis is optionally cell type-specific, as a result of which preference is given according to the invention to studying a plurality of cell lines and primary cells. Induction of apoptosis may optionally also be stimulus-specific. Therefore, preference is given to taking in a method of the invention a plurality of stress situations as a basis, for example heat shock, hypoxic conditions, cytokine treatments (e.g. IL-1, IL-6, TNF-alpha) or H₂O₂ treatment. Typical cell types suitable for such a method of the invention are customary cell lines, for example Cos cells, HEK cells, PC12 cells, THP-1 cells, or primary cells such as, for example, neurons, astrocytes, as well as other immortalized and primary cell lines, as required.

The present invention further relates to the use of nucleic acids of the invention, nucleic acid constructs of the invention or gene products of the invention for carrying out a proliferation assay and/or to methods of this kind using the aforementioned subject matters of the invention. Analogously, as for apoptosis assays above, it is possible, for example, to study the involvement of ee3 sequences, in particular ee3_{—}1 or ee3_{—}2, and of native or non-native variants thereof in cell growth, in cell cycle progress or in tumorigenic transformation by transfecting expression constructs containing ee3 polynucleotides of the invention, for example ee3_{—}1 or ee3_{—}2, or corresponding variants into eukaryotic cells and subsequently studying, for example, induction of tumorigenicity, for example with the aid of a soft-agar assay (Housey, et al., 1988, Adv. Exp. Med. Biol., 235, 127-40). Preferred suitable cell types are customary lines, for example Cos cells, HEK cells, PC12 cells, THP-1 cells, or primary cells such as, for example, neurons, astrocytes, as well as other immortalized and primary cell lines, as required. In particular, it is possible to study with the aid of such a method of the invention the function of gene products of the invention on the ras signal transduction pathway and the interaction of gene products of the invention with other components of the ras signal transduction pathway, in particular with regard to proliferative processes.

The present invention further relates to the use of a DNA sequence as claimed in any of claims 1 to 4 or of a gene product as claimed in any of claims 8 to 10 as a suicide gene/suicide protein for in vivo or ex vivo transformation of host cells. It is possible to specifically trigger in this way cell death in host cells, in particular with regard to the biological function of protein of the invention in signal transduction of apoptotic and/or necrotic signals. Preference is given here to designing the use of a DNA sequence of the invention and/or a protein of the invention so as for the suicide gene to be operatively linked to a promoter, with transcription being repressed and activated only when needed. In particular, it is possible, after transplanting patient cells, to switch off specifically the transfected cell ex vivo or in vivo in the course of a gene therapy.

In summary, it can be concluded that according to the invention a novel family of membrane-bound G protein-coupled receptors (GPCRs) has been identified in the mammalian system, which can be clearly distinguished from the families known from the prior art. A novel protein class and the underlying DNA sequences were identified according to the invention, owing to differential regulation thereof in the central nervous system, allowing to elucidate and characterize a multiplicity of physiological and pathophysiological processes.

The identification was carried out according to the invention by (directly or indirectly) EPO-induced transcriptional upregulation of the protein ee3_{—}1 of the invention, meaning that, for example, agonists and antagonists of ee3_{—}1 are capable of enhancing or replacing EPO actions or antagonizing undesired actions. Particular EPO actions may possibly be selectively influenced, for example a neuroprotective action (e.g. in neurodegeneratove disorders), or an increase in brain function (e.g. in dementias).

The gene presented here is a novel 7-transmembrane protein in mice and humans, which is expressed primarily in the brain. It is a G protein-coupled receptor.

Homology screening in the EMBL sequence database produced a distant similarity to GPCRs of the A family, in particular to peptide receptors.

In addition, ee3_{—}1 is regulated only in a limited way, if at all, by the following neurological disease models: kindling (hippocampus, seizure stage 5, 2 h postseizure), cortical stroke (cortex, 2.5 h occlusion and 2 and 6 h of reperfusion), global ischemia in rats (total brain, 3 and 6 h postischemia). This indicates a high specificity of regulation by EPO, in contrast to immediate early genes, for example.

The following figures illustrate the present invention in more detail:

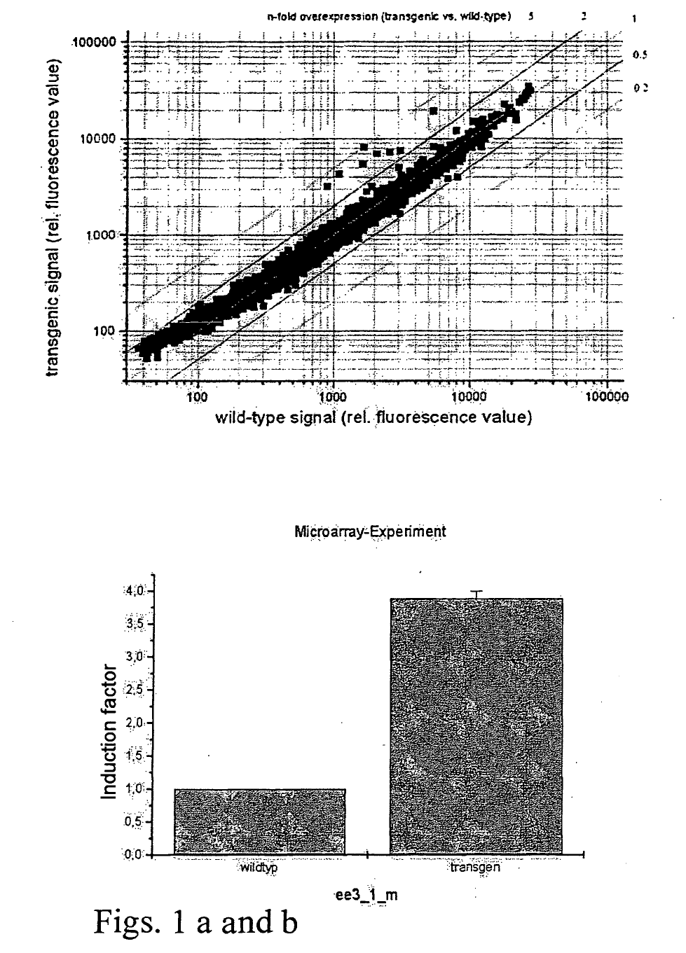

FIG. 1a depicts a representation of transcriptional analysis in the brain of Epo mice. The graph shows the data of a DNA array hybridization experiment. The signal in the EPO-transgenic animals (y axis) is plotted as a function of the signal in wildtype mice (x axis). The signal is a (relative) fluorescence signal. The points above the diagonal represent highly regulated gene products in the brain of EPO-transgenic animals. Eight positive signals can be observed above diagonal 2 (2-fold overexpression in the transgenic animal compared to the WT). FIG. 1b depicts the results of microarray experiments. Here, the (rel.) induction factor of murine ee3_{—}1 in the brain of EPO-transgenic mice (right-hand side) is plotted in relation to the induction in the brain of WT animals, namely as averaged induction values from 2 independent hybridization experiments. An induction factor of 1 corresponds to the concentration in the brain of the littermate control animals. Expression of a sequence of the invention is almost four times as high.

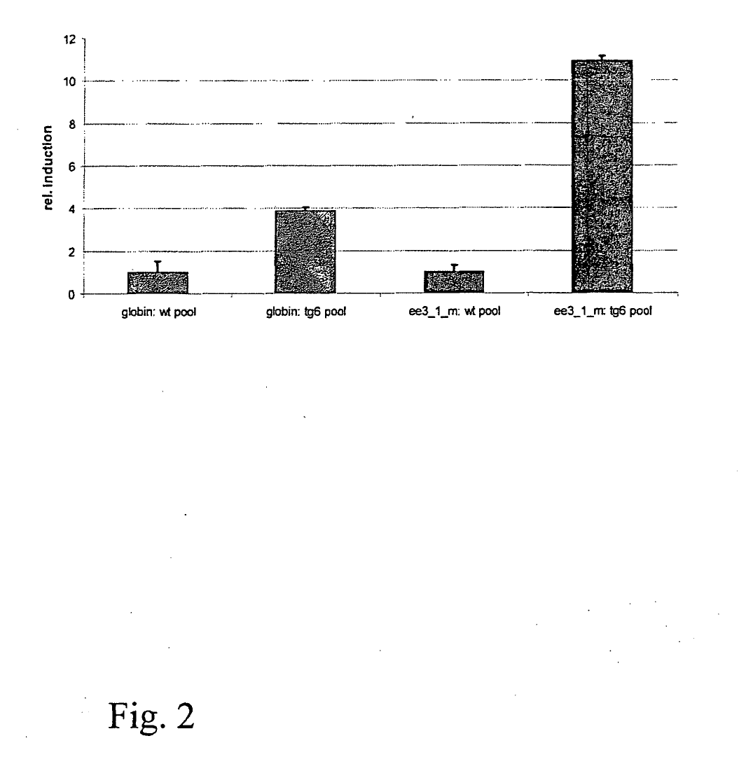

FIG. 2 depicts the results of experiments with mice, which were used to verify the increased induction of ee3_{—}1 of the invention in the EPO-transgenic animal and of the alpha-globin gene product which is likewise upregulated in the EPO-transgenic animal, this being done with the aid of quantitative PCR (LightCycler). The data represent pooled RNA samples from 6 brains (transgenic (tg) or wildtype (wt)). Relative induction is plotted along the y axis. Compared to the control measurement (rel. induction=1), the sequence of the invention results in an 11-fold increase in induction.

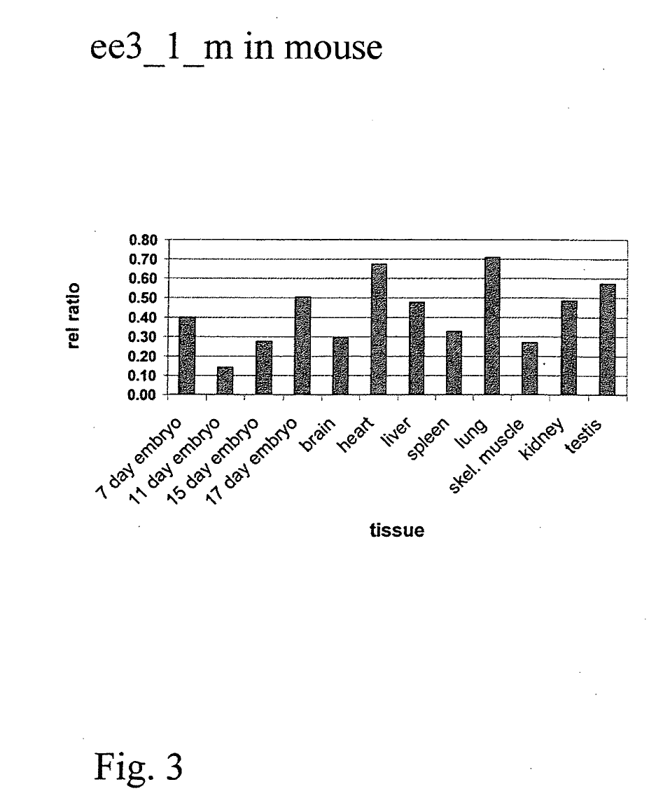

FIG. 3 represents expression of ee3_{—}1 of the invention in mice (LightCycler) during development (embryo after 7, 11, 15 and 17 days) and in various adult tissues (brain, heart, liver, kidney, lung, skeletal muscle, spleen and testis). The relative abundance of ee3 transcripts is plotted along the y axis. The result is a relatively ubiquitous expression of ee3_{—}1 in all stages of murine embryonic development and in all tissues studied.

According to EST data, ee3_{—}2 is expressed in mice in embryonic carcinoma, kidney, liver, B cells, lung, mamma and uterus.

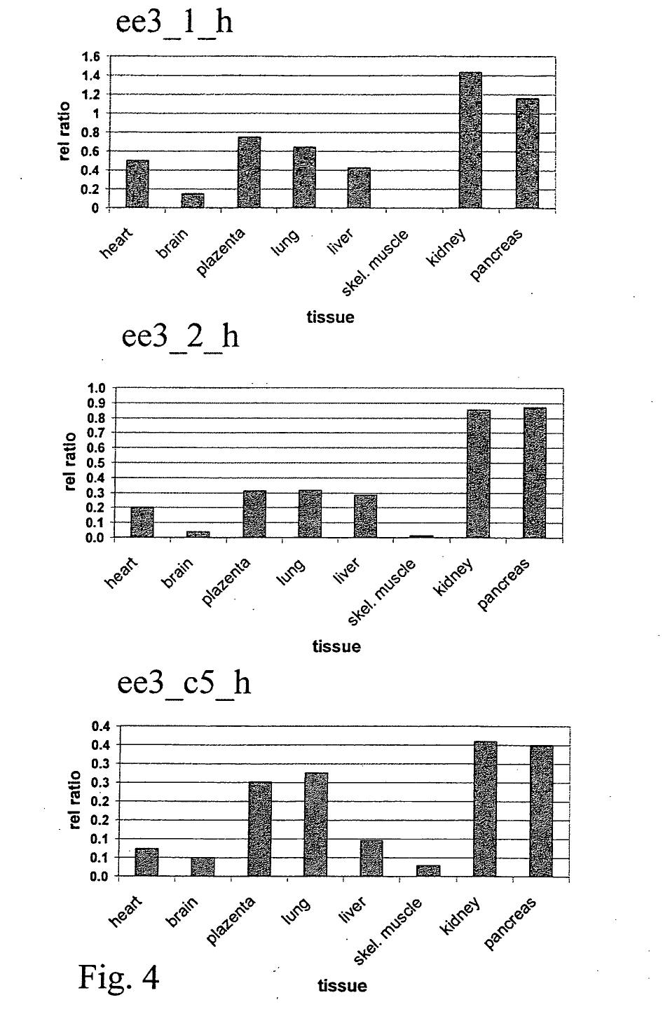

FIG. 4 depicts expression of human ee3_{—}1, ee3_{—}2 and ee3_c5 of the invention in humans in adult tissues (heart, brain, placenta, lung, liver, skeletal muscle, kidney, pancreas). Data are from quantitative PCR experiments (LightCycler). The values plotted along the y axis correspond to those in FIG. 3, revealing virtually ubiquitous and parallel expression of genes of the ee3 family of the invention in the tissues studied. Strongly increased expression of the ee3 family in the kidneys and the pancreas is particularly prominent, while expression in the brain and in skeletal muscle is lower. ee3_{—}1 expression and ee3_{—}2 expression are substantially identical so that a redundant function of these two proteins of the invention may be assumed.

In humans, ESTs with ee3_{—}1 sequences can be found in the following organs: brain, eye, germ cells, heart, kidney, lung, placenta, prostate, whole embryo, adrenal gland, mamma, colon, stomach, testis, indicating relatively broad expression. ESTs of ee3_{—}2 can be found in humans in the brain, colon, heart, kidney, lung, pancreas, parathyroid, prostate, testis, uterus, bladder, mamma, skin.

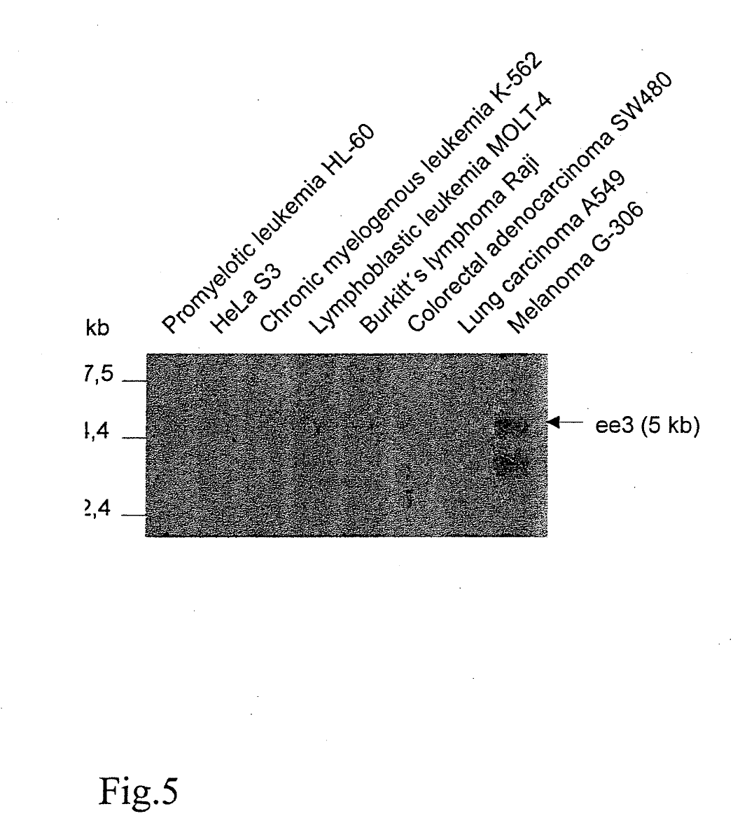

FIG. 5 represents the result of a Northern blot of expression of human ee3_{—}1 of the invention in various human tumor cell lines. A mouse probe comprising the ORF of human ee3_{—}1 was used for hybridization on said Northern blot (Clontech). Ubiquitous expression of a human ee3_{—}1 RNA transcript of the invention is revealed.

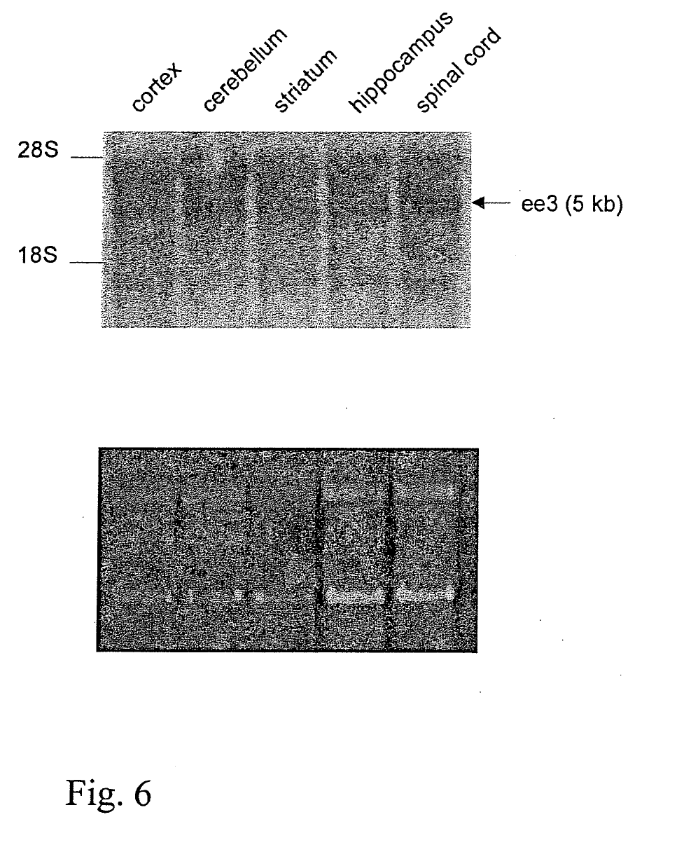

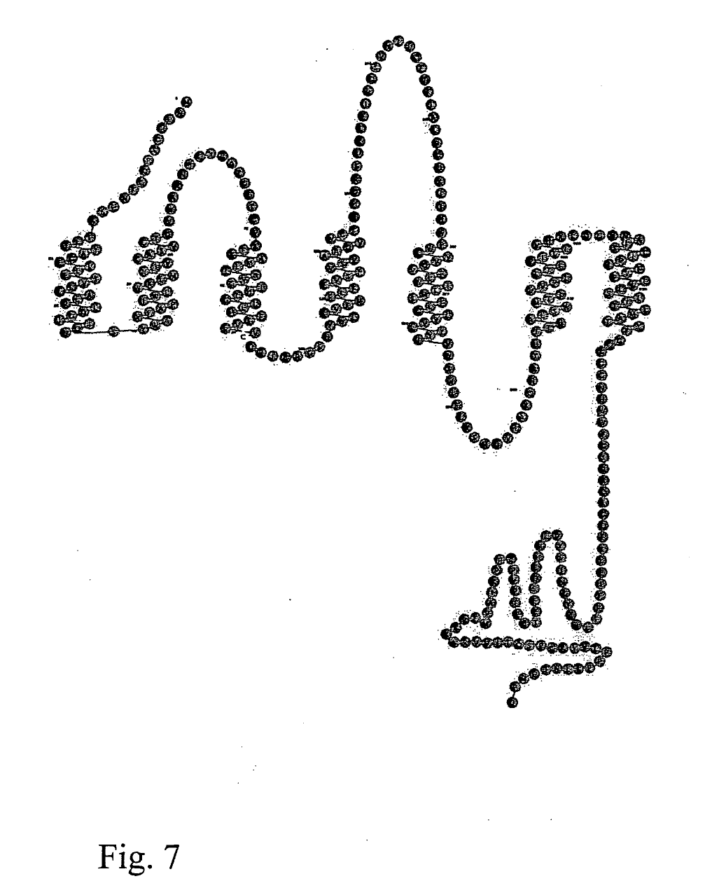

FIG. 6 depicts expression of ee3_{—}1 in various areas of the brain (rat). Here too, a ubiquitous distribution in various areas of the brain is found, which is somewhat stronger in the cerebellum and the spinal cord. Probe used: mouse ee3_{—}1. Shown underneath is the image of the ethidium bromide-stained gel as a loading control.

FIG. 7 depicts a model of the protein topology of ee3_{—}1_m on the basis of structural predictions with particular consideration of the transmembrane domains (TM domains). Said model reveals a typical topology of GPCR proteins, having 7 TM domains (depicted horizontally side by side), a short extracellular N terminus (located above TM domain 1) and an intracellular C terminus (depicted below TM domain 7). Hydrophobic amino acids are indicated in green.

FIG. 8 depicts an alignment (sequence comparison) of inventive proteins ee3_{—}1 (“human pro”), ee3_{—}2 and a protein fragment of ee3_{—}5 with various GPCR proteins previously known from the prior art (e.g. dc32_bio, ccr5-human or dop21_human) and consensus motifs of the GPCR families A, B and C known according to the prior art (as cons fam A, cons fam B in FIG. 8). “Pfam” means protein family and describes a group of consensus motifs resulting from the clustering of proteins. The motifs listed have been taken from the pfam databases. The inventive family of ee3 proteins clearly is most similar to the GPCR proteins of family A.

The following sequence sections for human ee3_{—}1 and ee3_{—}2 (subsequent AA numbering corresponds to that of FIG. 8) are particularly characteristic for the protein family of the invention in comparison with previously known GPCR proteins: AA 75-85, AA 129-135 (in particular glycine and serine in positions 129 and 132, respectively, glycine in position 174, AA 193-200 (in particular glycine in position 198), AAs in positions 260 and 261, AA in position 308 (Cys), AA 334-340, AA in position 539 (His), AA in position 608 (His), AA in position 611 (Asp), and finally the entire C-terminal sequence section from position 637, (in particular comprising the acidic motif between positions 640 and 655, the basic motif between 666 and 670 and the proline-rich motif between positions 680 and 685).

FIG. 9A depicts a murine DNA sequence of the invention (sequence 1), referred to as ee3_c1 (ee3_{—}1), which comprises the translated region (all sequences shown are read in the following way: continuously from left to right and from top to bottom, i.e. continuing from the end of the line to the line immediately below, left). The start codon and the stop codon in this sequence region are highlighted in bold type. FIG. 9B comprises another sequence of the invention, which is a subregion (in the 3′ untranslated region) of the sequence according to FIG. 9A.

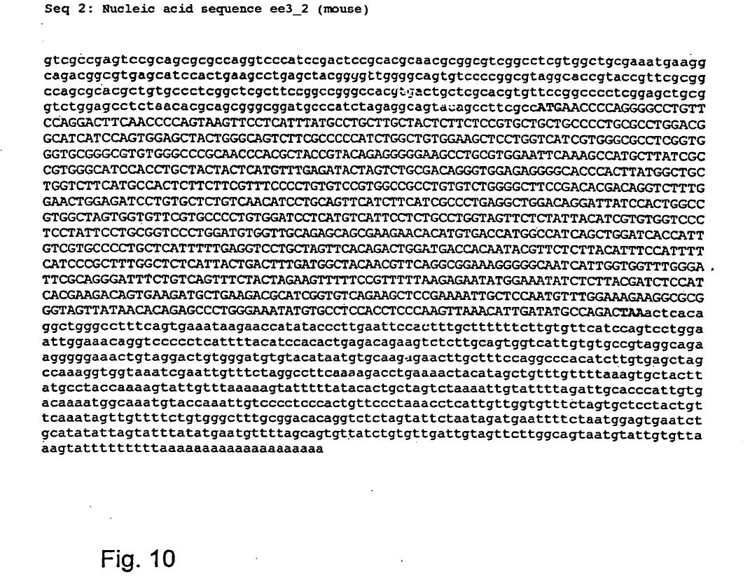

FIG. 10 represents a murine DNA sequence of the invention (sequence 2) referred to as ee3_{—}2, which also includes the translated region. The start codon and the stop codon in this sequence region are highlighted in bold type.

FIG. 11A represents a human DNA sequence of the invention (sequence 3) referred to as ee3_{—}1, which also includes the translated region. The start codon and the stop codon in this sequence region are highlighted in bold type. In addition, the putative polyadenylation signal is highlighted in bold type and by underlining. FIGS. 11B and 11C depict alternative C-terminal splice forms coding for a C-terminally truncated protein of the invention. In addition to the start and stop codons highlighted in bold type in both figures, FIG. 11B also contains the highlighted consensus sequence of the splice site.

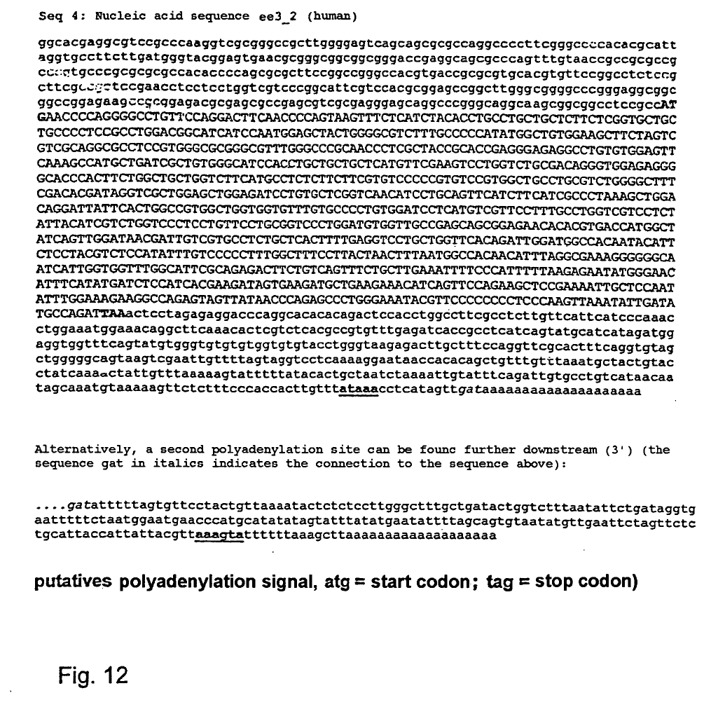

FIG. 12 represents a human DNA sequence of the invention (sequence 4) referred to as ee3_{—}2, which also includes the translated region. The start codon and the stop codon in this sequence region are highlighted in bold type (ATG and TAA, respectively). In addition, the putative polyadenylation signal is highlighted in bold type. The bottom sequence in FIG. 12 is a continuation of the first part of the sequence (overlapping region of the first and, respectively, the second part in italics).

FIG. 13 represents a murine AA sequence of the invention, referred to as ee3_{—}1 (sequence 5), running continuously from the N terminus to the C terminus (see also underlying DNA sequence according to FIG. 9).

FIG. 14 represents a murine AA sequence of the invention, referred to as ee3_{—}2 (sequence 6), running continuously from the N terminus to the C terminus (see also underlying DNA sequence according to FIG. 10).

FIG. 15A represents a human AA sequence of the invention, referred to as ee3_{—}1 (sequence 7), running continuously from the N terminus to the C terminus (see also underlying DNA sequence according to FIG. 11A). FIG. 15B represents a human AA sequence of the invention, referred to as ee3_{—}1b_h (sequence 7b), running continuously from the N terminus to the C terminus (see also underlying DNA sequence according to FIG. 11B). FIG. 15C represents a human AA sequence of the invention, referred to as ee3_{—}1c_h (sequence 7c), running continuously from the N terminus to the C terminus (see also underlying DNA sequence according to FIG. 11C). The sequences according to FIGS. 15B and 15C are the AA sequences of alternative splice products of the DNA sequence depicted in FIG. 11A.

FIG. 16 represents a human AA sequence of the invention, referred to as ee3_{—}2 (sequence 8), running continuously from the N terminus to the C terminus (see also underlying DNA sequence according to FIG. 12).

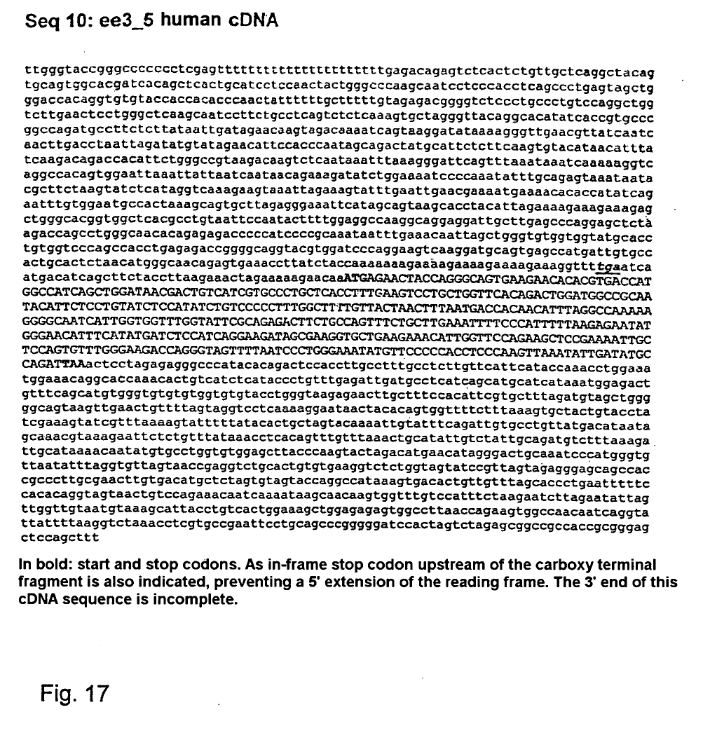

FIG. 17 depicts a human cDNA sequence of the invention (sequence 10) referred to as ee3_{—}5, which also includes the translated region. The start codon and the stop codon (ATG and TAA, respectively), in this sequence region are highlighted in bold type.

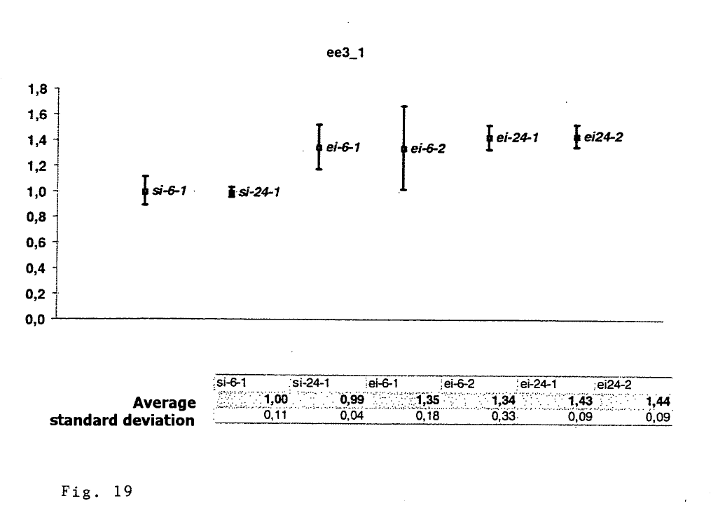

FIG. 18 represents a human AA sequence of the invention, referred to as ee3_{—}5 (sequence 11), running continuously from the N terminus to the C terminus (see also underlying DNA sequence according to FIG. 19).

FIG. 19 depicts the result of a quantitative PCR for ee3_{—}1 in the brains of mice which were treated intraperitoneally with 5000 U of erythropoietin (EPO)/kg of body weight and, 6 or 24 hours thereafter, perfused and studied. si-6-1, si-24-1: animals injected with saline, after 6 and 24 hours, respectively. ei-6-1, ei-6-2: animals injected with EPO, after 6 hours; ei-24-1, ei-24-2: animals injected with EPO, after 24 hours. An increase in ee3 RNA-expression is revealed, said expression increasing with time. The data of the EPO-treated animals differ in a statistically significant manner from those of the saline-treated animals (ANOVA, followed by Newman-Keuls post hoc test).

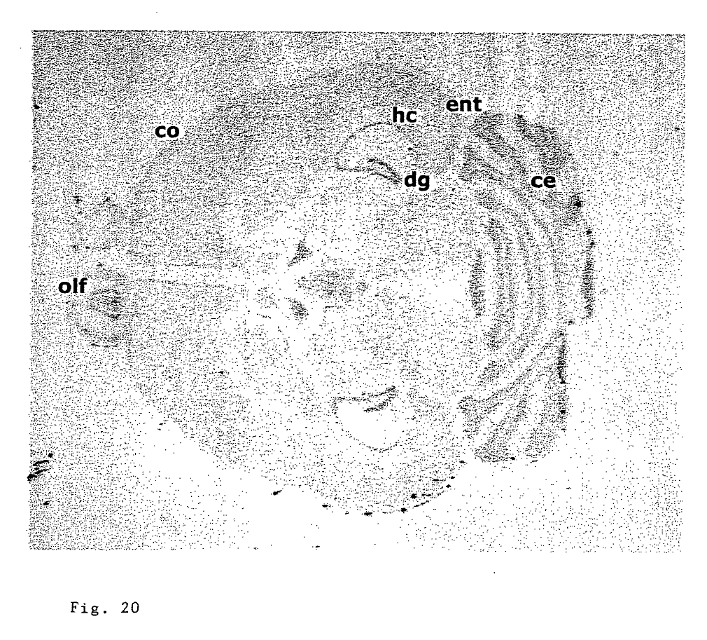

FIG. 20 depicts the image of an in situ hybridization on a horizontal section through a mouse brain. Using the radiolabeled probe (ee3_{—}1.3 as AACGAAGGGCCAGTAGCACAGAGAACAGCAGCAGACAGGCATAGATGAGG), it was possible to visualize expression of ee3_{—}1 in the cerebellum (ce), hippocampus (hc), dentate gyrus (dg) and in the cortex (co), in particular in the entorhinal cortex (ent), in the olfactory bulb (olf). A corresponding sense control (ee3-1.3s, CCTCATCTATGCCTGTCTGCTGCTGTTCTCTGTGCTACTGGCCCTTCGTT) gave no specific signal (not shown).

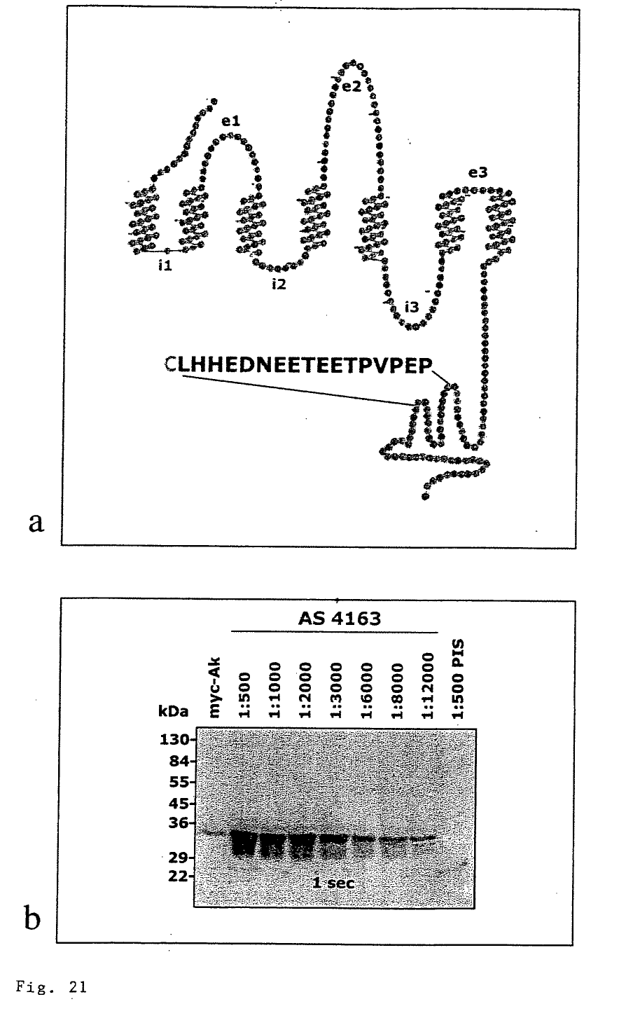

FIG. 21 illustrates the preparation of a C-terminal polyclonal antiserum against the ee3_{—}1 protein (human). a: Selection of a peptide epitope on the carboxy terminus, having high antigenicity potential (CLHHEDNEETEETPVPEP). b: Immunoblot depicting the specific detection of ee3_{—}1 in transiently transfected HEK293 cells. In each case, the same amounts of lysate from HEK293 cells transfected with the construct Exp.ee3-1-h-Nter-myc, resulting in production of ee3_{—}1 protein with N-terminally fused myc tag, were applied. Lane 1: detection of the ee3_{—}1 protein with N-terminally fused myc tag via a myc-specific antibody (Upstate Biotechnology (sold by Biomol Feinchemikalien GmbH), used in a dilution of 1:2000). Lanes 2-8: detection of ee3_{—}1, using different dilutions of the ee3_{—}1-specific antiserum AS4163 (1:500-1:12 000). The antiserum specifically detects in a highly sensitive manner the ee3_{—}1-specific band (approx. 35 kDa). Lane 9: the corresponding pre-immune serum (PIS), diluted 1:500, does not detect any band.

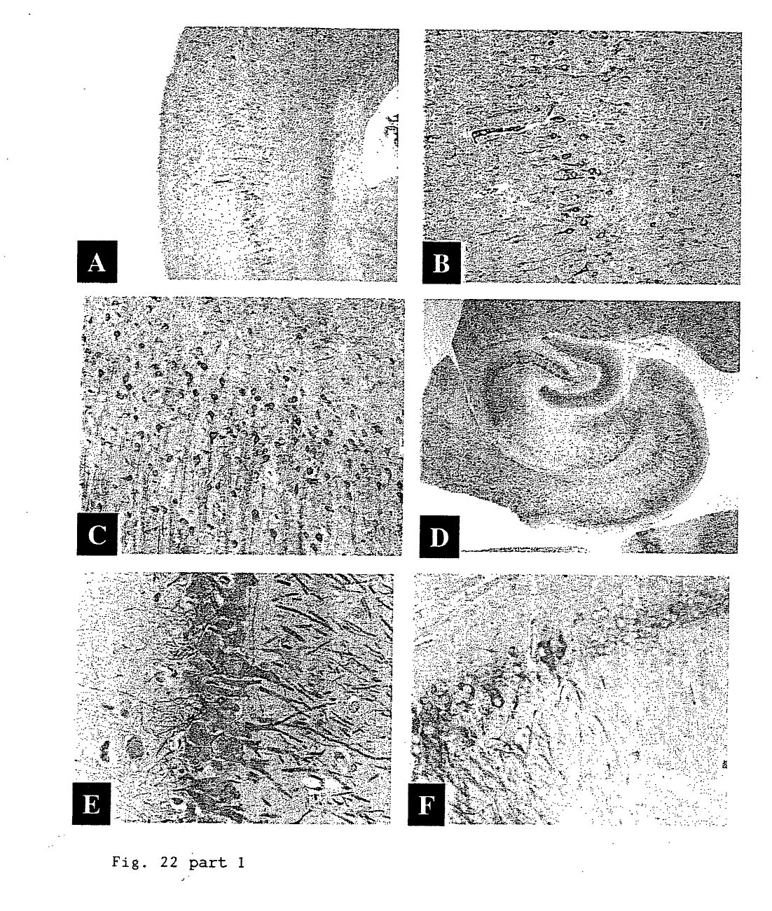

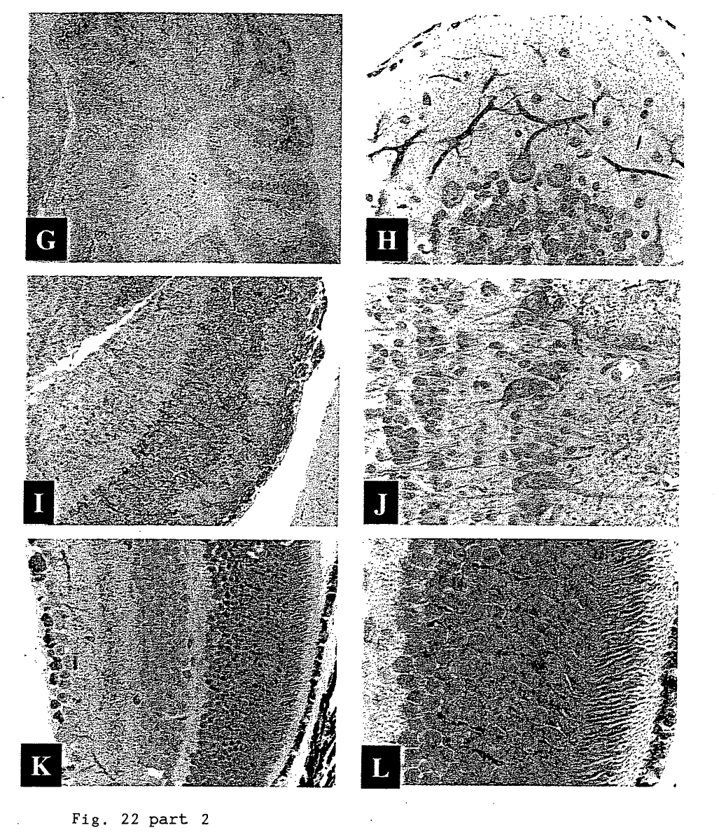

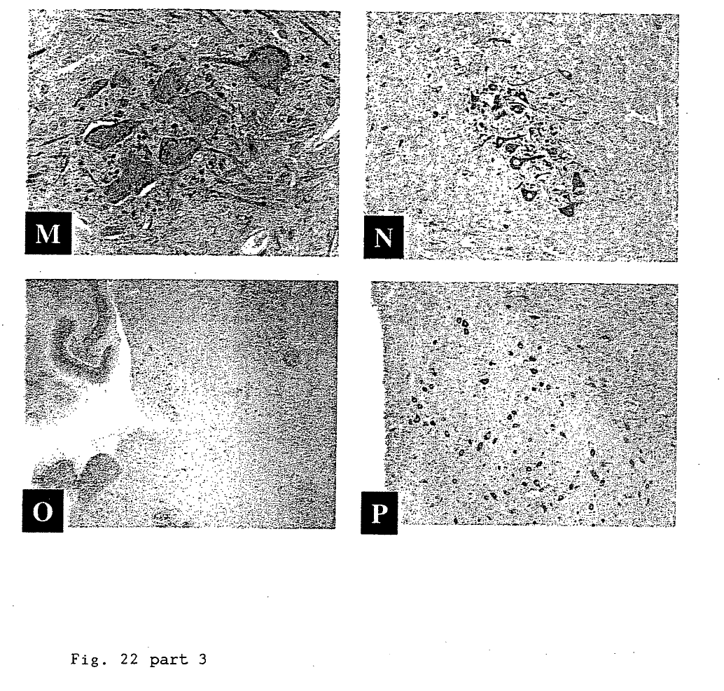

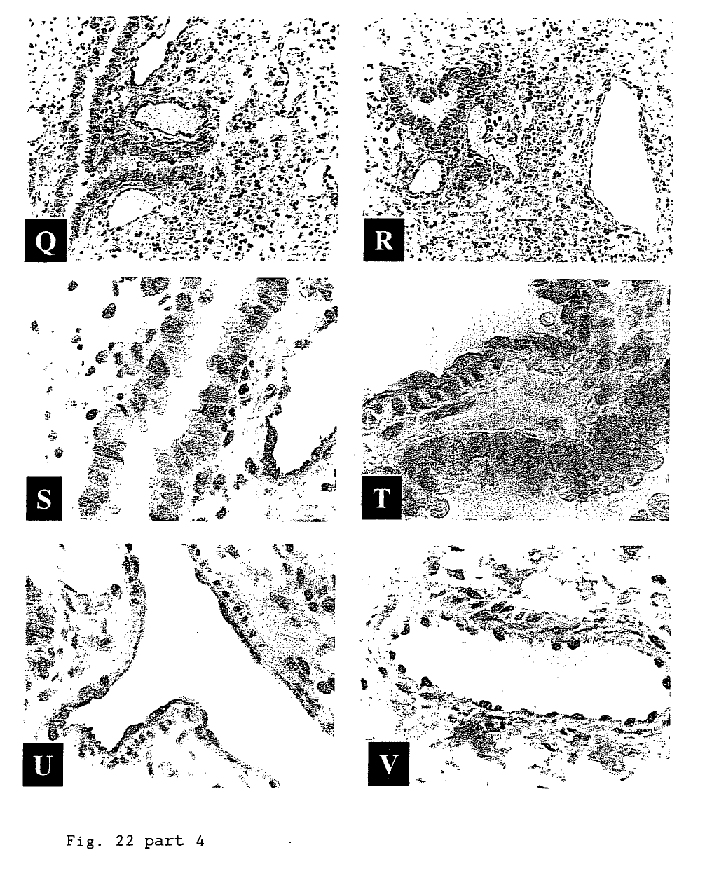

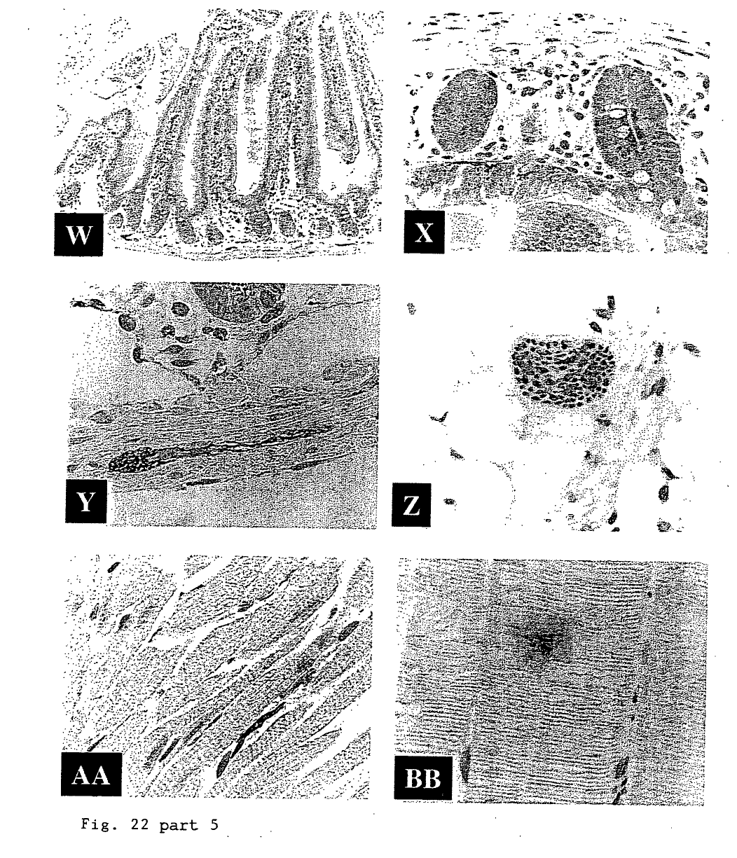

FIG. 22 depicts the immunohistochemical detection of ee3_{—}1 in various tissues by means of the AS4163 antiserum. A: specific staining of layer V neurons in the somatosensory cortex. B: enlargement of A. C: neurons in the entorhinal cortex. D: expression of ee3_{—}1 in the dentate gyrus and in the CA3 hippocampal region. E: magnification of the CA3 hippocampal region. F: boundary of ee3_{—}1 expression in the hippocampus between CA3 and CA2. G: cerebellum, specific immunohistochemical staining in the Purkinje cell layer and in cerebellar nuclei. H: Purkinje cells in the cerebellum. I: olfactory bulb. J: magnification, staining of large mitral cells. K: retina, staining of ganglial cells and sensory cells of the retina. L: magnification of K. Staining of the sensory cells of the retina. M: expression of ee3_{—}1 in the large motoneurons of the anterior horn in the spinal cord. N: expression of ee3_{—}1 in the motor nucleus of the trigeminal nucleus. O: staining of the substantia nigra, pars reticulata. P: magnification of Q. substantia nigra. Q: ee3_{—}1 is expressed extraneurally in the lung. Staining of basal cells in the bronchioli. Staining of the arterioles, no staining of the venoles. R: representation of the typical pulmonal trials bronchus, artery and vein. S: magnification of the bronchioli. Expression in specific basal cells not yet defined in more detail. T: magnification with arteriole wall (top left) and bronchiolus wall (bottom right). U: longitudinal section of an arteriole. Staining of the endothelium and of individual smooth muscle cells in the vascular wall. V: cross section of an arteriole with immunohistochemical staining of smooth muscle cells and of individual endothelial cells. W: small intestine with cryptal and villous structures. Staining of basal crypt portions by the antibody against ee3_{—}1. Individual vegetative nerves are stained in the villi. X: magnification of W. Y: representation of nerve fibers in the wall of the small intestine, which belong to the vegetative myenteric plexus of the intestine. Z: cross section of a peripheral nerve in subcutaneous fatty/connective tissue. AA: heart muscle with specifically stained nerve fibers. BB: striated muscles (skeletal muscle). The immunohistochemical staining in the center of the image is highly consistent with a motor end plate. Individual peripheral nerve fibers in the perimysium (bottom right).

FIG. 23 depicts an immunohistochemical staining of ee3_{—}1 in the spinal cord of a wildtype mouse (top part of the image) in comparison with that in the spinal cord of a mouse transgenically overexpressing erythropoietin [(tg6) lower part of image]. A distinctly stronger signal is found in the transgenic mice under identical staining conditions. This finding was verified using in each case two further mice.

FIG. 24 depicts a double immunofluorescence for ee3_{—}1 and map1b in mice. Said two proteins were detected as interaction partners in a y2h system. The locations of the two proteins in the CNS were found to correspond to an astonishing degree. Green: ee3_{—}1 staining; red: Map1b staining; yellow: electronic superimposition of both signals. Examples from the spinal cord (sc) and from the cerebellum (cb) are shown.

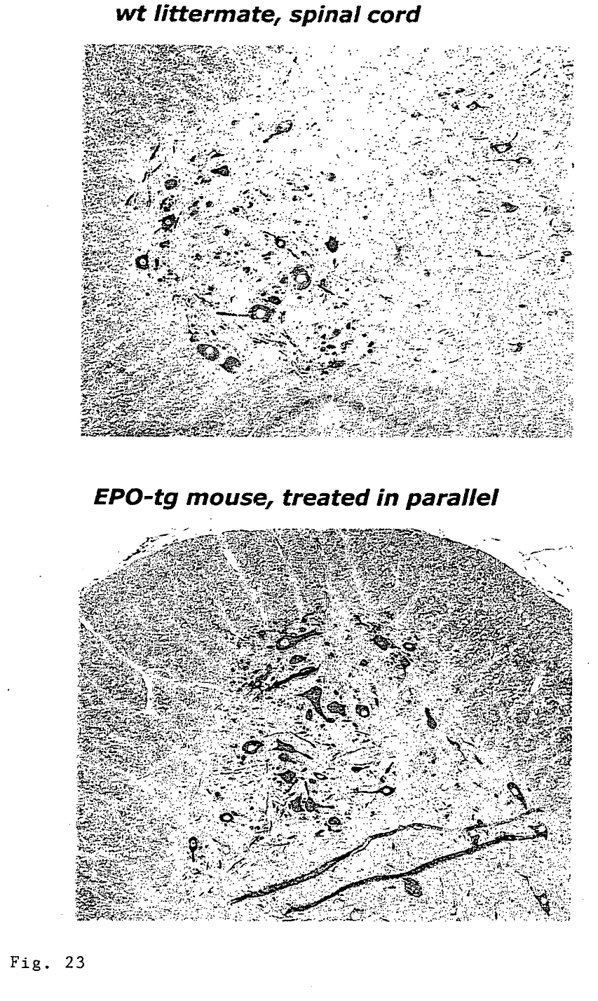

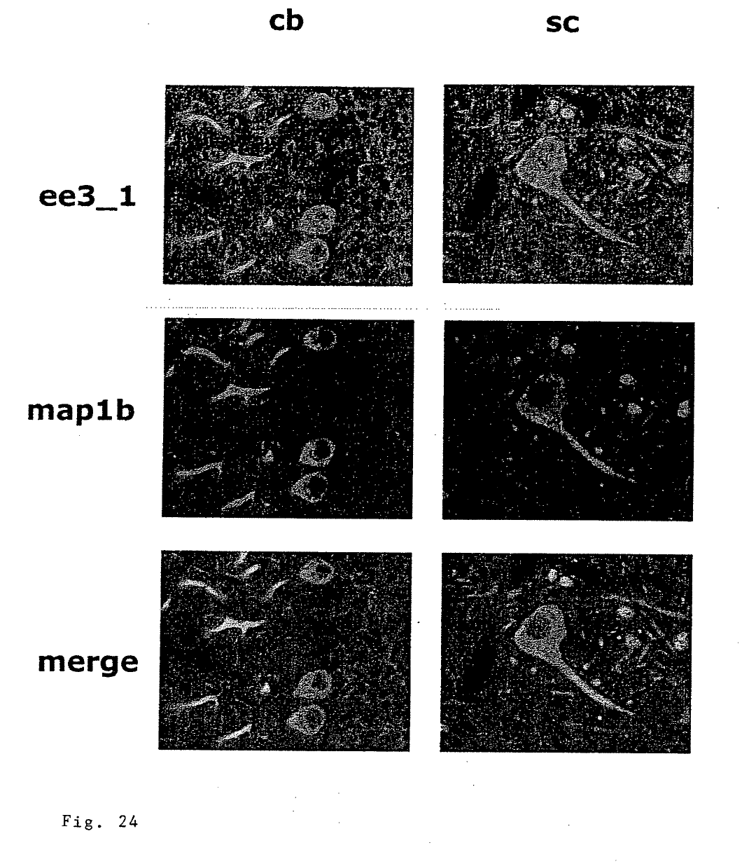

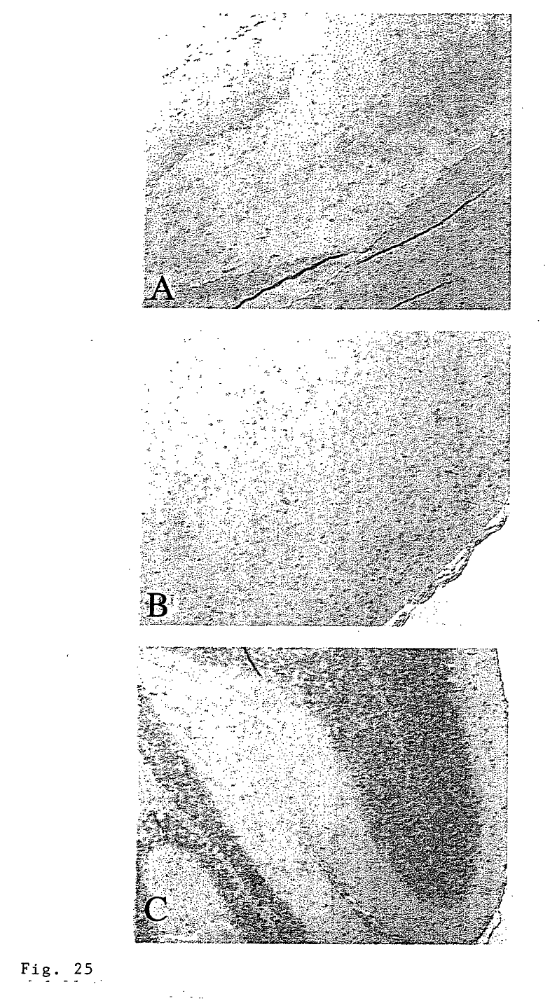

FIG. 25 depicts immunohistochemical stainings of a mouse mutant for the map1b gene (Meixner, et al. (2000), J. Cell Biol., 151, 1169-78, revealing that only traces of ee3_{—}1 can still be found in the map1b-homozygous ko animals. a: hippocampus, b: cortex, c: cerebellum.

FIG. 26 depicts a PCR for ee3_{—}1 in adult neural stem cells (nsc) from rat hippocampus. No signal can be detected in the negative lane (N). ee3_{—}1 is expressed by these neural stem cells.

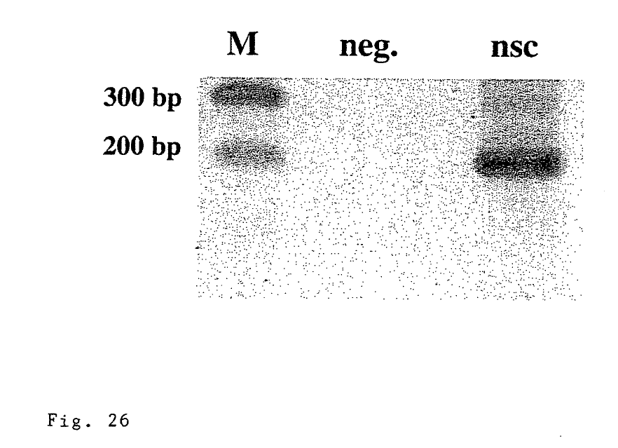

FIG. 27 depicts the protein alignment of ee3 proteins from various species, taking into account the sequences from X. laevis and D. rerio.

The following exemplary embodiment illustrates the present invention in more detail:

### EXEMPLARY EMBODIMENT 1

Identification and Molecular Cloning of ee3_{—}1_m and Homologs

(a) Identification of ee3_{—}1_m

The brain of transgenic erythropoietin-overexpressing mice was removed under anesthetic after transcardial perfusion and shock frozen in liquid nitrogen. RNA was obtained according to the method of Chomczynski and Sacchi (Anal Biochem (1987), 162, 156-9). Hybridization experiments of 2 transgenic and 2 littermate controls on a mouse cDNA array (chip) were carried out according to the procedure of Incyte (see http://www.incyte.com/reagents/lifearray/lifearray service.s html). This involves carrying out competitive hybridization with the aid of two differently labeled samples (labeled with Cy5 and Cy3). The hybridization experiment produced a number of upregulated sequences. In particular, the EST clone AA185432 was identified which, in a repeat experiment, was likewise upregulated in the Epo-transgenic mice. The relative induction factor was +3.9±0.1 compared to the nontransgenic littermates (FIG. 1). Said upregulation was confirmed with the aid of a quantitative PCR using the LightCycler system (FIG. 2, forward primer: 5′-GGTGTGGGAGAAATGGCTTA-3′, reverse primer: 5′-ATACCAGCAGAGCCTGGAGA-3′).

(b) Cloning of ee3 Sequences

The identified EST sequence, was extended with the aid of BLASTN queries in EST databases. In this way, another homologous murine sequence, ee3_{—}2_m, was identified. By making use of homology screenings using appropriate programs (BLAST, TBLASTN), it was possible to identify human homologs in EST and genomic databases (ensembl).

The sequences obtained were confirmed by screening in murine and human sequence databases with the aid of the PCR cloning method of Shepard (Shepard A R, Rae J L (1997) Magnetic bead capture of cDNAs from double-stranded plasmid cDNA libraries. Nucleic Acids Res 25:3183-3185). The aforementioned publication and the prior art cited therein are incorporated in their entirety into the disclosure of the present invention. Said method is based on hybridizing cDNA molecules from a plasmid library to a biotin-coupled oligonucleotide sequence, subsequently extracting said plasmids with the aid of streptavidin-coupled magnetic beads, checking the result by means of diagnostic PCR and twice repeating said steps, after retransforming the plasmid selection obtained, until the single clones are obtained. The following primer combinations were used:

(1) oligonucleotides used for cloning the full-length gene section:

For ee3_1-h:
ee3_1-5′biotin1-hs:
AATTCCTCATCTATGCCTGTCTGCT
ee3_1-3′block1-hs:
GCTGTTCTCTGTGCTGCTGGCCCTTCGTTTGGATGGCATC
ee3_1-5′block1-hs:
ATGAACCTGAGGGGCCTCTTCCAGGACTTCAACCCGAGTA
ee3_1-1s-hs:
TGCTCCAATATGGCTGTGGA
ee3_1_1as-hs:
CTCTAGTGACCTGTCATGTC
ee3_1-2s-h:
GACAGAGCTTAAGTGGACTG
ee3_1-2as-h:
TACAGTTCCTACTGACTGCC
ee3_1-5′block2-h:
ACGCACTCTCTCCGCCTTCCTCTGCCCCCTCGTTCACCCC
ee3_1-5′biotin2-h:
GCAGACCAGAACCAGTACTGGAGCT
ee3_1-3′block2-h:
GGGTCTCCAGGTACGTCCATCTCATGCCTTGTTTGCATCC
For ee3_1-m
ee3_1-5′biotin1-m:
ATTCCTCATCTATGCCTGTCTGCTG
ee3_1-3′block1-m:
CTGTTCTCTGTGCTACTGGCCCTTCGTTTGGATGGCATCA
ee3_1-5′block1-m:
TGAACCTGAGGGGCCTCTTTCAGGACTTCAACCCGAGTAA
ee3_1-1s-m:
GGATGGCATCATTCAATGGAG
ee3_1-1as-m:
GAACAATGGCATGAAGACCAG
ee3_1-2s-m:
ACTGAGCTGGATGACCATTGT
ee3_1-2as-m:
TCCTCACTATCTTCATGGTGG
ee3_1-5′biotin2-m:
TCATCACCCAGAGCCCTGGCAAGTA
ee3_1-5′block2-m:
CCTAAAATTGCACCTATGTTCCGCAAGAAGGCCAGGGTAG
ee3_1-3′block2-m:
TGTCCTTCCTCCACCCAAACTAAATATTGAAATGCCAGAC
For ee3_2_h:
ee3_c2-1as-h:
TGAACTGCAGGATGTTGACC
ee3_c2-1s-h:
TCATCCAATGGAGCTACTGG
ee3_c2-5′block1-h:
ATGAACCCCAGGGGCCTGTTCCAGGACTTCAACCCCAGTA
ee3_c2-5′biotin1-h:
AGTTTCTCATCTACACCTGCCTGCT
ee3_c2-3′block1-h:
GCTCTTCTCGGTGCTGCTGCCCCTCCGCCTGGACGGCATC
For ee3_2-m:
ee3_c2-3′block1-m:
ACTCTTCTCCGTGCTGCTGCCCCTGCGCCTGGACGGCATC
ee3_c2-5′biotin1-m:
AGTTCCTCATTTATGCCTGCTTGCT
ee3_c2-1as-m:
TGGATAATCCTGTCCAGCCT
ee3_c2-1s-m:
ATCATCCAGTGGAGCTACTG
ee3_c2-5′block1-m:
ATGAACCCCAGGGGCCTGTTCCAGGACTTCAACCCCAGTA
ee3_c2-m-2s:
TGTGGAAGCTCCTGGTCATCGT
ee3_c2-m-2as:
GATAATCCTGTCCAGCCTCAGG
ee3_c2-5′block2-m:
GAAGCTCCTGGTCATCGTGGGCGCCTCGGTGGGTGCGGGC
ee3_c2-5′biotin2-m:
GTGTGGGCCCGCAACCCACGCTACC
ee3_c2-3′block2-m:
GTACAGAGGGGGAAGCCTGCGTGGAATTCAAAGCCATGCT
ee3_c2-5′biotin3-m:
ACAGAGCCCTGGGAAATATGTGCCT
ee3_c2-3′block3-m:
CCACCTCCCAAGTTAAACATTGATATGCCAGACTAAACTC
ee3_c2-5′block3-m:
TTGCTCCAATGTTTGGAAAGAAGGCGCGGGTAGTTATAAC
For ee3_c3-h:
ee3_c3-5′block1-h:
CCACCTTGGGCACCTTGGTGTCTTTCAAAAGTGCCAGGCT
ee3_c3-5′biotin1-h:
CCTTCCTGCCTCAGGGCCTTTGCAC
ee3_c3-3′block1-h:
TTGCTGCTCCCTCCGTTTGAAATACTGTATCCCAGAGAGT
ee3_c3-1s-h:
GGCACCTTGGTGTCTTTCAA
ee3_c3-1as-h:
CAGTCTGAATTAGGAGCCAG
For ee3_c5-h:
ee3_c5-1as-h:
TCGGAGCTTCTGGAACCAAT
ee3_c5-1s-h:
CCATCAGCTGGATAACGACT
ee3_c5-5′block1-h:
ACCATGGCCATCAGCTGGATAACGACTGTCATCGTGCCCC
ee3_c5-5′biotin1-h:
TGCTCACCTTTGAAGTCCTGCTGGT
ee3_c5-3′block1-h:
TCACAGACTGGATGGCCGCAATACATTCTCCTGTATCTCC
For ee3_c8-h:
ee3_c8-5′block1-h:
AATTTTGGTATATGGTGCAAAAAAAGGGGTCCAATTTCTT
ee3_c8-5′biotin1-h:
CTGCAACTGGCCAGCCAGTTATCTC
ee3_c8-3′block1-h:
AGCATCATTAATTGAATAGGGAATCCTTACCCCACTGATT
ee3_c8-1s-h:
AACTGGCCAGCCAGTTATCT
ee3_c8-1as-h:
AATGGATTGTTGGGTGCAGC
ee3_c8-2s-h:
CCAGCCAGTTATCTCAGCATCA
ee3_c8-2as-h:
ACCATGGCATGTGTATCCCAGA

(2) In addition, the coding region of the ee3 sequences was cloned into GATEWAY™-compatible vectors in order to be able to carry out functional analyses. The following oligonucleotides were used for this:

For ee3_1-h:
ee3_1_h_B1:
GGGG ACA AGT TTG TAC AAA AAA GCA GGC
TACCATGAACCTGAGGGGCCTCTTCCA
ee3_1_h_B2:
GGGG AC CAC TTT GTA CAA GAA AGC TGG GTC
CTAATCTGGCATTTCGATATTTAATTTGGGAGGT
ee3_1-h-C-fus-B2:
GGGG AC CAC TTT GTA CAA GAA AGC TGG GTC
GCATTTCGATATTTAATTTGGGAGGTGGGAG
For ee3_2-h:
ee3_c2-h-B1:
GGGG ACA AGT TTG TAC AAA AAA GCA GGG TCTACC
ATGAACCCCAGGGGCCTGTTCC
ee3_c2-h-B2:
GGGG AC CAC TTT GTA CAA GAA AGC TGG GTC
TTAATCTGGCATATCAATATTTAACTTGGGAGGG
ee3_c2-h-c-fus-B2:
GGGG AC CAC TTT GTA CAA GAA AGC TGG GTC
ATCTGGCATATCAATATTTAACTTGGGAGGG
For ee3_c2-m:
ee3_c2-m-B1:
GGGG ACA AGT TTG TAC AAA AAA GCA GGC
TCTACCATGAACCCCAGGGGCCTGTTCC
ee3_c2-m-B2:
GGGG AC CAC TTT GTA CAA GAA AGC TGG GTC
TTAGTCTGGCATATCAATGTTTAACTTGGGAG

(c) Preparation of the Human cDNA Library

Starting from 2 μg of human fetal brain mRNA (Clontech, Heidelberg, Germany) and from 5 μg of mRNA from adult mouse brain, corresponding cDNA libraries were prepared using the cDNA synthesis kit from Stratagene (Amsterdam, the Netherlands). The procedure was carried out essentially according to the manufacturer's instructions. First strand cDNA synthesis was carried out using an oligodT primer according to the manufacturer's instructions. The cloning-compatible (EcoRI/XhoI) double-stranded cDNA fragments were selected according to size (according to the manufacturer's instructions/Stratagene) and ligated into the plasmid vector pBluescript SKII (Stratagene). The ligation was transformed by way of electroporation into E. coli (DH10B, Gibco) and amplified on LB-ampicillin agar plates. The plasmid DNA was isolated by means of alkaline lysis and ion exchange chromatography (QIAfilter kit from Qiagen, Hilden, Germany).

The complexity of individual clones for the fetal human brain cDNA bank was 4 million. 24 single clones of each cDNA bank were randomly analyzed according to insert size and displayed a size distribution of from 800 bp up to 4.5 kB, the average length of the cDNA insert for the human bank being approx. 1.2 kB.

### EXEMPLARY EMBODIMENT 2

Regulation of ee3_{—}1 by Erythropoietin (EPO)

ee3_{—}1 was identified as an upregulated gene product in brains of Epo-transgenic mice (murine lines tg6 and tg21).

The mice used for the experiments of the invention have previously been characterized several times with respect to their constitution (Ruschitzka et al., 2000, Proc Natl Acad Sci USA, 97, 11609-13.; Wagner et al., 2001, Blood, 97, 536-42.; Wiessner et al., 2001, J Cereb Blood Flow Metab, 21, 857-64.). The mice were prepared using a transgenic construct according to the method described in Hergersberg (Hergersberg et al., Hum. Mol. Genet. 4, 359-366). This construct comprised a PDGF promoter and the sequence coding for erythropoietin. A plurality of transgenic lines was produced, of which tg6 and tg21 were studied here. Only tg6 had systemically increased EPO expression which was confirmed by serum studies according to the method of Ruschitzka et al., (2000, Proc Natl Acad Sci USA, 97, 11609-13). The line tg21 had no increased systemic EPO levels. In analogy to the results of Sasahara et al., (1991, Cell, 64, 217-27.), the PDGF-promoter fragment used may be assumed to cause expression of the transgenic EPO, especially in neuronal cells.

In mice of the tg6 line, increased systemic expression of EPO results in a distinct increase in erythropoiesis, leading to polyglobulism up to a hematocrit of 0.8 and a distinctly increased blood volume (up to 4.0 ml) (Wagner et al., 2001, Blood, 97, 536-42). In contrast, the tg21 line is phenotypically not very conspicuous.

The RNA products, for example of the ee3_{—}1 gene, were increasingly expressed in the brain of mice transgenically overexpressing erythropoietin (lines tg6 and tg21 (Ruschitzka et al., 2000, Proc Natl Acad Sci USA, 97, 11609-13., Wagner et al., 2001, Blood, 97, 536-42., Wiessner et al., 2001, J Cereb Blood Flow Metab, 21, 857-64.)) and were identified by way of a DNA array experiment. The physiological and pathophysiological importance of transcriptional EE3_{—}1 regulation by overexpression of EPO was confirmed by finding another regulator gene product, namely alpha-globin, which was likewise found to be regulated in both transgenic lines with the aid of a transcription analysis using microarrays. This was confirmed with the aid of the LightCycler system (FIG. 2). The increase was visible especially in hippocampal areas (in situ hybridization).

### EXEMPLARY EMBODIMENT 3

Expression of Sequences of the Invention in Mammalian Cells and Preparation of Stable Cell Lines

The open reading frame of the genes of the ee3 family was cloned into a common eukaryotic expression vector of the pcDNA series from Clontech (Heidelberg, Germany). The expression plasmids being produced in this way were used to transfect human embryonic kidney cells (HEK293), in particular by the calcium phosphate method, CHO cells and CHO-dhfr⁻ cells by means of lipofectamine or COS cells by means of DEAE-dextran beads, and selected using 400-500 mg/ml G418. Three weeks after selection, individual clones were picked and expanded for further analysis. Approximately 30 clones were analyzed by Northern blot and Western blot methods. Transfected CHO-dhfr⁻ cells were selected in nucleotide-free medium by cloning the open reading frame of the genes of the ee3 family into a eukaryotic expression vector containing the dihydrofolate reductase gene as selectional marker and by using the resulting expression plasmid for transfection. CHO-dhfr⁻ cells transfected in this way, but also other cells transfected in this way, may be treated with increasing concentrations of methotrexate and were treated in this way, thereby selecting cells which express increased amounts of dihydrofolate reductase and thus also increased amounts of receptor.

### EXEMPLARY EMBODIMENT 4

Yeast 2-Hybrid Experiment Using a Carboxy-Terminal Section of ee3_{—}1

To identify in the yeast 2-hybrid system potential interaction partners, the carboxy-terminal part of ee3_{—}1 of the invention was cloned into the bait vector pGBKT7 (Clontech).

The protein sequence used was:

KGGNHWWFGIRKDFCQFLLEIFPELREYGNISYDLHHEDNEETEETPVPE
PPKIAPMFRKKARVVITQSPGKYVLPPPKLNIEMPD,

The corresponding nucleic acid sequence was:

AAGGGAGGAAACCACTGGTGGTTTGGTATCCGCAAAGATTTCTGTCAGTT
TCTGCTTGAAATCTTCCCATTTCTACGAGAATATGGAAACATTTCCTATG
ATCTCCATCACGAAGATAATGAAGAAACCGAAGAGACCCCAGTTCCGGAG
CCCCCTAAAATCGCACCCATGTTTCGAAAGAAGGCCAGGGTGGTCATTAC
CCAGAGCCCTGGGAAGTATGTGCTCCCACCTCCCAAATTAAATATCGAAA
TGCCAGAT

The screening for interaction partners was carried out using a human brain library and according to standard methods familiar to the skilled worker (mating methods, Clontech). As a result, 2 clones (clones 11 and 36) were obtained which included overlapping sequences.

The sequence in the identified clone 11 was as follows:

GGGGACTCGGCCCTGAACGAGCAGGAGAAGGAGTTGCAGCGGCGGCTGAA
GCGTCTNTACCCGGCCGTGGACNAACAAGAGACGCCGTTGCCTCGGTCCT
GGAGCCCGAAGGACAAGTTCAGCNTACATCGGCCTNTNTNAGAACAACCT
GCGGGTGCACTACAAAGGTCATGGCAAAACCCCAAAAGATGCCGCGTCAG
TTCGAGCCACGCATCCAATACCAGCAGCCTGTGGGATTTATTATTTTGAA
GTAAAAATTGTCAGTAAGGGAAGAGATGGTTNCATGGGAATTGGTCTTTC
TGCTCAAGGNGTGAACATGAATAGACTACCAGGTTGGGATAAGCATTCAT
ATGGTTACCATGGGGATGATGGACATTCGTTTTGTTCTTCTGGAACTGGA
CAACCTTATGGACCAACTTTCACTACTGGTGATGTCATTGGCTGTTGTGT
TAATCTTATCAACAATACCTGCTTTTACACCAAGAATGGACATAGTTTAG
GTATTGCTTTCACTGACCTACCGCCAAATTTGTATCCTACTGTGGGGCTT
CAAACACCAGGAGAAGTGGTCGATGCCAATTTTGGGCAACATCCTTTCGT
GTTTGATATAGAAGACTATNTGCGGGAGTGGAGAACCAAAATCCAGGCNC
AGATAGATCGATT.

The interacting gene product was identified as RANBPM or RANBP9 (Nishitani H, Hirose E, Uchimura Y, Nakamura M, Umeda M, Nishii K, Mori N, Nishimoto T (2001) Full-sized RanBPM cDNA encodes a protein possessing a long stretch of proline and glutamine within the N-terminal region, comprising a large protein complex. Gene 272:25-3). Likewise, two other interacting proteins were identified, namely Map1a and Map1b. Interestingly, the carboxy-terminal part in both proteins was identified as being the interacting part. said part contains a homologous region in both proteins. An alignment of Map1a and Map1b in this region is shown, the top sequence being Map1a and the bottom sequence being Map1b:

```
ALIGN calculates a global alignment of two sequences
version 2.0uplease cite: Myers and Miller, CABIOS
(1989) 4:11-17
Sequence 1 212 aa vs.
Sequence 2 177 aa
scoring matrix: BLOSUM50, gap penalties: −12/−2
43.6% identity; Global alignment score: 614
                              10 20 30 40 50
/tmp/f KEKVQGRVGRRAPGKAKPASPARRLDLRGKRSPTPGKGPADRASRAPPRP--RSTTSQVT
                                                                                      : ...:.. : ... ..
Sequen -----------------------------------KKESVEKAAKPTTTPEVKAARGEEK
                                                                                                    10 20
              60 70 80 90 100 110
/tmp/f PAEEKDGHSPMSKGLVNGLKAGPMALSSKGSS----GAPVYVDLAYIPNXCSGKTADLDF
                  : :.. . .. .. ::: . .. :: : :::.:: :::: ..:..:..:
Sequen DKETKNAANASASKSAKTATAGPGTTKTTKSSAVPPGLPVYLDLCYIPNHSNSKNVDVEF
                    30 40 50 60 70 80
                    120 130 140 150 160 170
/tmp/f FRRVRASYYVVSGNDPANGXPSRAVLDALLEGKAQWGENLQVTLIPTHDTEVTREWYQQT
              :.:::.::::::::::: ::::::::::::::::: :.:::::::::.:: :::::.:
Sequen FKRVRSSYYVVSGNDPAAEEPSRAVLDALLEGKAQWGSNMQVTLIPTHDSEVMREWYQET
                    90 100 110 120 130 140
                    180 190 200 210
/tmp/f HEQQQQLNVLVLASTXTVVMQDESFPACRLSSEKPPSL
              ::.::.::..::::. ::::::::::::..
Sequen HEKQQDLNIMVLASSSTVVMQDESFPACKIEL------
                  150 160 170
```

### EXEMPLARY EMBODIMENT 5

Human Homologous Sequences of ee3_{—}1/ee3_{—}2

(a) On Chromosome 5q33.1

Another homologous sequence was determined on contig AC11406.00015 with the aid of Tblastn:

```
>AC011406.00015
Length: 40,820
Minus Strand HSPs:
Score = 389 (136.9 bits), Expect = 1.3e − 41, Sum P(3) = 1.3e − 41
Identities = 72/303 (71%), Positives = 78/303 (77%), Frame = −1
Query: 224 LLTFEILLVHKLDGHNAFSCIPIFVPLWLSLITLMATTFGQKGGNHWWFGIRKDFCQFLL 283
                          LLTFE+LLVH+LDG N FSCI I VPLWL L+TLM TTF K GNHWWFGIR+DFCQFLL
Sbjct: 14312 LLTFEVLLVHRLDGRNTFSCISISVPLWLLLLTLMTTTFRPKRGNHWWFGIRRDFCQFLL 14253
Query: 284 EIFPFLREYGNISYDLHHEDNXXXXXXXXXXXXKIAPMFRK 324
                          EIFPFLREYGNISYDLH ED+ KIAP+F K
Sbjct: 14252 EIFPFLREYGNISYDLHQEDSEGAEETLVPEAPKIAPVFGK 14010
Score = 86 (30.3 bits), Expect = 1.3e− 41, Sum P(3) = 1.3e− 41
Identities = 15/51 (88%), Positives = 16/51 (94%), Frame = −3
Query: 334 PGKYVLPPPKLNIEMPD 350
                          PGKYV PPPKLNI+MPD
Sbjct: 13992 PGKYVPPPPKLNIDMPD 13942
Score = 67 (23.6 bits), Expect 1.3e − 41, Sum P(3) = 1.3e− 41
Identities = 12/57 (63%), Positives 17/57 (89%), Frame = −2
Query: 206 QRRTHITMALSWMT-IVVP 223
                          Q RTH+TMA+SW+T ++VP
Sbjct: 14368 Q*RTHVTMAISWITTVIVP 14312
```

It was possible to obtain the corresponding cDNA, but translation results only in a carboxy-terminal fragment homologous to the ee3 proteins.

Sequence comparison with ee3_{—}1_m is as follows:

```
ALIGN calculates a global alignment of two sequences
version 2.OuPlease cite: Myers and Miller, CABIOS (1989)
4:11-17
Sequence 1 350 aa vs.
Sequence 2 148 aa
scoring matrix: BLOSUM50, gap penalties: −12/−2
29.4% identity; Global alignment score: 649
                              10 20 30 40 50 60
/tmp/f MNLRGLFQDFNPSKFLIYACLLLFSVLLALRLDGIIQWSYWAVFAPIWLWKLMVIVGASV
Sequen ------------------------------------------------------------
                              70 80 90 100 110 120
/tmp/f GTGVWARNPQYRAEGETCVEFKAMLIAVGIHLLLLMFEVLVCDRIERGSHFWLLVFMPLF
Sequen ------------------------------------------------------------
                            130 140 150 160 170 180
/tmp/f FVSPVSVAACVWGFRHDRSLELEILCSVNILQFIFIALRLDKIIHWPWLVVCVPLWILMS
Sequen ------------------------------------------------------------
                            190 200 210 220 230 240
/tmp/f FLCLVVLYYIVWSVLFLRSMDVIAEQRRTHITMALSWMTIVVPLLTFEILLVHKLDGHNA
                                              .:. .. .. : : :. :::::.::::.:::.:.
Sequen ----------------MRTTRAV-KNTRDH-GHQLD-NDCHRALLTFEVLLVHRLDGRNT
                                                                10 20 30 40
                            250 260 270 280 290 300
/tmp/f FSCIPIFVPLWLSLITLMATTFGQKGGNHWWFGIRKDFCQFLLEIFPFLREYGNISYDLH
              :::: : ::::: :.:::.::: : ::::::::.::::::::::::::::::::::::
Sequen FSCISISVPLWLLLLTLMTTTFRPKRGNHWWFGIRRDFCQFLLEIFPFLREYGNISYDLH
                            50 60 70 80 90 100
                            310 320 330 340 350
/tmp/f HEDSEETEETPVPEPPKIAPMFRKKARVVITQSPGKYVLPPPKLNIEMPD
              .:::: .::: ::: :::::.: :.:::. ::::: :::::::.:::
Sequen QEDSEGAEETLVPEAPKIAPVF-GKTRVVLI--PGKYVPPPPKLNIDMPD
                          110 120 130 140
```

The generation of only one GPCR fragment is certain, since the cDNA sequences obtained totally correspond to genomic data and exhibit the presence of an in-frame stop codon upstream of the ATG (see sequence):

```
Minus Strand HSPs:
Score = 6976 (1046.7 bits), Expect 0.0, Sum P(2) = 0.0
Identities = 1396/1397 (100%), Positives = 1396/1397 (100%),
Strand = Minus/Plus
Query: 2499 AGGTTTAGACCTTAAAATAATACCTGATTGTTGGCCACTTCTGGTTAAGGCCACTCTCTC 2440
                          ||||||||||||||||||||||||||||||||||||||||||||||||||||||||||||
Sbjct: 13048 AAGTTTAGACCTTAAAATAATACCTGATTGTTGGCCACTTCTGGTTAAGGCCACTCTCTC 13107
Query: 2439 CAGCTTTCCAGTGACAGGTAATGCTTTACATTACAACCAACTAATATTCTAAGATTCTTA 2380
                          ||||||||||||||||||||||||||||||||||||||||||||||||||||||||||||
Sbjct: 13108 CAGCTTTCCAGTGACAGGTAATGCTTTACATTACAACCAACTAATATTCTAAGATTCTTA 13167
Query: 2379 GAAATGGACAAACCACTTGTTGCTTATTTTGATTGTTTCTGGACAGTTACTACCTGTGTG 2320
                          ||||||||||||||||||||||||||||||||||||||||||||||||||||||||||||
Sbjct: 13168 GAAATGGACAAACCACTTGTTGCTTATTTTGATTGTTTCTGGACAGTTACTACCTGTGTG 13227
Query: 2319 GAAAAATTCAGGGTGCTAAACAACAGTGTCACTTTATGGCCTGGTACTACACTAGAGCAT 2260
                          ||||||||||||||||||||||||||||||||||||||||||||||||||||||||||||
Sbjct: 13228 GAAAAATTCAGGGTGCTAAACAACAGTGTCACTTTATGGCCTGGTACTACACTAGAGCAT 13287
Query: 2259 GTCACAAGTTCGCAAGGGCGGTGGCTGCTCCCTCTACTAACGGATACTACCAGAGACCTT 2200
                          ||||||||||||||||||||||||||||||||||||||||||||||||||||||||||||
Sbjct: 13288 GTCACAAGTTCGCAAGGGCGGTGGCTGCTCCCTCTACTAACGGATACTACCAGAGACCTT 13347
Query: 2199 CACACAGTGCAGACCTCGGTTACTAACACCTAAATATTAACACCCATGGGATTTGCAGTC 2140
                          ||||||||||||||||||||||||||||||||||||||||||||||||||||||||||||
Sbjct: 13348 CACACAGTGCAGACCTCGGTTACTAACACCTAAATATTAACACCCATGGGATTTGCAGTC 13407
Query: 2139 CCTATGTTCATGTCTAGTACTTGGGTAAGCTCCACACCAGGCACATATTGTTTTATGCAA 2080
                          ||||||||||||||||||||||||||||||||||||||||||||||||||||||||||||
Sbjct: 13408 CCTATGTTCATGTCTAGTACTTGGGTAAGCTCCACACCAGGCACATATTGTTTTATGCAA 13467
Query: 2079 TCTTTAAAGACATCTGCAATAGACAATATGCAGTTTAAACAAACTGTGAGGTTTATAAAC 2020
                          ||||||||||||||||||||||||||||||||||||||||||||||||||||||||||||
Sbjct: 13468 TCTTTAAAGACATCTGCAATAGACAATATGCAGTTTAAACAAACTGTGAGGTTTATAAAC 13527
Query: 2019 AGAGAATTCTTTACGTTTGCTATTATGTCATAACAGGCACAATCTGAAATACAATTTTGT 1960
                          ||||||||||||||||||||||||||||||||||||||||||||||||||||||||||||
Sbjct: 13528 AGAGAATTCTTTACGTTTGCTATTATGTCATAACAGGCACAATCTGAAATACAATTTTGT 13587
Query: 1959 ACTAGCAGTGTATAAAAATACTTTTAAACGATACTTTCGATAGGTACAGTAGCACTTTAA 1900
                          ||||||||||||||||||||||||||||||||||||||||||||||||||||||||||||
Sbjct: 13588 ACTAGCAGTGTATAAAAATACTTTTAAACGATACTTTCGATAGGTACAGTAGCACTTTAA 13647
Query: 1899 AGAAAACCACTGTGTAGTTATTCCTTTTGAGGACCTACTAAAACAGTTCAACTTACTGCC 1840
                          ||||||||||||||||||||||||||||||||||||||||||||||||||||||||||||
Sbjct: 13648 AGAAAACCACTGTGTAGTTATTCCTTTTGAGGACCTACTAAAACAGTTCAACTTACTGCC 13707
Query: 1839 CCCAGCTACATCTAAAGCACGAATGTGGAAAGCAAGTTCTCTTACCCAGGTACACACCAC 1780
                          ||||||||||||||||||||||||||||||||||||||||||||||||||||||||||||
Sbjct: 13708 CCCAGCTACATCTAAAGCACGAATGTGGAAAGCAAGTTCTCTTACCCAGGTACACACCAC 13767
Query: 1779 ACACACCCACATGCTGAAACAGTCTCCATTTATGATGCATGCTGATGAGGCATCAATCTC 1720
                          ||||||||||||||||||||||||||||||||||||||||||||||||||||||||||||
Sbjct: 13768 ACACACCCACATGCTGAAACAGTCTCCATTTATGATGCATGCTGATGAGGCATCAATCTC 13827
Query: 1719 AAACAGGGTATGAGATGACAGTGTTTGGTGCCTGTTTCCATTTCCAGGTTTGCTATGAAT 1660
                          ||||||||||||||||||||||||||||||||||||||||||||||||||||||||||||
Sbjct: 13828 AAACAGGGTATGAGATGACAGTGTTTGGTGCCTGTTTCCATTTCCAGGTTTGGTATGAAT 13887
Query: 1659 GAACAAGAGGCAAAGGCAAGGTGGAGTCTGTGTATGGGCCCTCTCTAGGAGTTTAATCTG 1600
                          ||||||||||||||||||||||||||||||||||||||||||||||||||||||||||||
Sbjct: 13888 GAACAAGAGGCAAAGGCAAGGTGGAGTCTGTGTATGGGCCCTCTCTAGGAGTTTAATCTG 13947
Query: 1599 GCATATCAATATTTAACTTGGGAGGTGGGGGAACATATTTCCCAGGGATTAAAACTACCT 1540
                          ||||||||||||||||||||||||||||||||||||||||||||||||||||||||||||
Sbjct: 13948 GCATATCAATATTTAACTTGGGAGGTGGGGGAACATATTTCCCAGGGATTAAAACTACCT 14007
Query: 1539 GGTCTTCCCAAACACTGGAGCAATTTTCGGAGCTTCTGGAACCAATGTTTCTTCAGCACC 1480
                          ||||||||||||||||||||||||||||||||||||||||||||||||||||||||||||
Sbjct: 14008 GGTCTTCCCAAACAGTGGAGCAATTTTCGGAGCTTCTGGAACCAATGTTTCTTCAGCACC 14067
Query: 1479 TTCGCTATCTTCCTGATGGAGATCATATGAAATGTTCCCATATTCTCTTAAAAATGGGAA 1420
                          ||||||||||||||||||||||||||||||||||||||||||||||||||||||||||||
Sbjct: 14068 TTCCCTATCTTCCTGATGGAGATCATATGAAATGTTCCCATATTCTCTTAAAAATGGGAA 14127
Query: 1419 AATTTCAAGCAGAAACTGGCAGAAGTCTCTGCGAATACCAAACCACCAATGATTGCCCCT 1360
                          ||||||||||||||||||||||||||||||||||||||||||||||||||||||||||||
Sbjct: 14128 AATTTCAAGCAGAAACTGGCAGAAGTCTCTGCGAATACCAAACCACCAATGATTGCCCCT 14187
Query: 1359 TTTTGGCCTAAATGTTGTGGTCATTAAAGTTAGTAACAAAAGCCAAAGGGGGACAGATAT 1300
                          ||||||||||||||||||||||||||||||||||||||||||||||||||||||||||||
Sbjct: 14188 TTTTGGCCTAAATGTTGTGCTCATTAAAGTTACTAACAAAAGCCAAAGGGGGACAGATAT 14247
Query: 1299 GGAGATACAGGAGAATGTATTGCGGCCATCCAGTCTGTGAACCAGCAGGACTTCAAAGGT 1240
                          ||||||||||||||||||||||||||||||||||||||||||||||||||||||||||||
Sbjct: 14248 GGAGATACAGGAGAATGTATTGCGGCCATCCAGTCTGTGAACCAGCAGGACTTCAAAGGT 14307
Query: 1239 GAGCAGGGCACGATGACAGTCGTTATCCAGCTGATGGCCATGGTCACGTGTGTTCTTCAC 1180
                          ||||||||||||||||||||||||||||||||||||||||||||||||||||||||||||
Sbjct: 14308 GAGCAGGGCACGATGACAGTCGTTATCCAGCTGATGGCCATGGTCACGTGTGTTCTTCAC 14367
Query: 1179 TGCCCTGGTAGTTCTCATTTGTTCTTTTTCTAGTTTCTTAAGGTAGAAGCTGATGTCATT 1120
                          ||||||||||||||||||||||||||||||||||||||||||||||||||||||||||||
Sbjct: 14368 TGCCCTGGTAGTTCTCATTTGTTCTTTTTTCTAGTTTCTTAAGGTAGAAGCTGATGTCATT 14427
Query: 1119 GATTCAAAACCTTTCTT 1103
                          |||||||||||||||||
Sbjct: 14428 GATTCAAAACCTTTCTT 14444
```

(b) On Chromosome 8q11.22

Another homologous sequence is found on Ensembl contig Ac034174.

The protein sequence of a homologous nucleotide section is as follows:

AATTTTGGTATATGGTGCAAAAAAAGGGGTCCAATTTCTTCTGCAACTGG
CCAGCCAGTTATCTCAGCATCATTAATTGAATAGGGAATCCTTACCCCAC
TGATTGTTTTTGTCAGGTTTGTCAAAGATGAAATAGTTGTAGGTGTATGG
TCTTATTTCTGGGTTCCCCATTCTGTTCCACTGGTATATGTGTCTGGTTT
TGAACTAGTGCCATGCTGTTTTGGTTACTATAGCCCTGTTTAAAATCAAA
TGGAGTGATGCCGCCACGTTGTATTTATTTTATTTTTTTATTATACTTTA
AGTTCTGGGATACACATGCCATGGTGGTTTGCTGCACCCAACAATCCATT
ATCTATGTTGTTTTCTC

(c) On Chromosome 3p25.3

A homologous sequence is found on chromosome 3:

CCACCTTGGGCACCTTGGTGTCTTTCAAAAGTGCCAGGCTCCTTCCTGCC
TCAGGGCCTTTGCACTTGCTGCTCCCTCCGTTTGAAATACTGTATCCCAG
AGAGTCCCATTTCTGGCTCCTAATTCAGACTGA

(d) On Chromosome Xp21.1:

```
>AC027722.00010
Length: 3,576
Minus Strand HSPs:
Score = 65 (22.9 bits), Expect = 4.1e + 02, Sum P(2) = 1.0
Identities = 11/90 (37%), Positives = 19/90 (63%), Frame = −1
Query: 159 RLDKIIHWPWLVVCVPLWI-LMSFLCLVVL 187
                        R+ ++W L C+P+W+ +SF CL+ L
Sbjct: 1848 RIMSSLNWDSLTSCLPIWMTFISFSCLIAL 1759
Score = 57 (20.1 bits), Expect = 4.1e + 02, Sum P(2) = 1.0
Identities = 16/147 (33%), Positives = 24/147 (49%), Frame = −2
Query: 88 VGIHLLLLMFEVLVCDRIERF-SH-FWLLVFMPLFFVSPVSVAACVWGF 134
                        +G L++ F V D++ G H FW L +PLF VS C + +
Sbjct: 2507 IGCCFLIVCFVCFVEDQMYVGLQHYFWALYSVPLFCVSVFVPVPCSFSY 2361
```

Nucleotide sequences corresponding to these homologous sections are as follows:

ATCATGTCATCTCTAAACTGGGATAGTTTGACTTCCTGTCTTCCTATTTG
GATGACTTTTATTTCTTTCTCTTGCCTGATTGCTCTGG
and
ATAGGGTGTTGTTTCCTCATTGTTTGTTTTGTCTGCTTTGTGGAAGATCA
GATGTATGTAGGTTTGCAGCATTATTTCTGGGCTCTCTATTCTGTTCCTT
TGTTCTGTGTGTCTGTGTTTGTACCAGTACCATGTTCTTTTAGTTACT

The consensus sequence DRI, however, is missing.

(d) Alternative Splice Products ee3_{—}1b_h and ee3_{—}1c_h

An alternative splice product of the human gene product ee3_{—}1_h is found, namely ee3_{—}1b_h (see FIG. 11B, sequence number 3B). Said product results from a consensus splice-donor site in exon 3 and results in a modified open reading frame having a modified carboxy terminus and an earlier translation stop. This results in a protein (166 amino acids, molecular weight: 19.2 kD) which stops after the fourth TM domain (see FIG. 15B, sequence number 7B). In addition, a further alternative splice product was identified, namely ee3_{—}1c_h (see FIG. 11C, sequence number 3C), with the corresponding protein sequence according to FIG. 15C (sequence number 7C) which is truncated after the second TM domain. Said splice products were identified in the course of the cloning and sequencing procedures with the aid of the cloning method of Shepard et al. (see example 1).

A prediction of the TM regions for ee3_{—}1b_h is as follows:—

-----> STRONGLY preferred model: N-terminus outside
4 strong transmembrane helices, total score: 6315

| # | from | to | length | score | orientation |
|---|---|---|---|---|---|
| 1 | 15 | 33 | (19) | 2066 | o-i (o: outside, i: inside) |
| 2 | 40 | 56 | (17) | 1143 | i-o |
| 3 | 83 | 102 | (20) | 1765 | o-i |
| 4 | 112 | 134 | (23) | 1341 | i-o. |

This splice product is functionally important in regulating the function of the full-length receptors, cf., for example, V2 vasopressin receptor (Zhu and Wess, 1998, Biochemistry, 37, 15773-84; Schulz, et al., 2000, J Biol Chem, 275, 2381-9)). Since GPCR proteins are subject to homo- or heterodimerizations (Bouvier, 2001, Nat Rev Neurosci, 2, 274-86.), such truncated forms of sequences of the invention may play a dominant-negative part.

As a result, the present invention discloses in particular the use of such splice forms (for example as naked DNA, in an expression vector of the invention, as protein sequence of the invention, etc.) of ee3 proteins of the invention and also of variants of such splice forms for preparing drugs for the treatment of disorders, as disclosed herein. The disclosure likewise comprises also their use for studying the ability of inventive receptors of the ee3 family to be pharmacologically influenced.

### EXEMPLARY EMBODIMENT 6

Protein Topology Data of Proteins of the ee3 Family

A TM (transmembrane) screening using the TMPred program results in the following strongly favored model:

(a) ee3_1
-----> STRONGLY preferred model: N-terminus outside
7 strong transmembrane helices, total score: 11863

| | | | | | |
|---|---|---|---|---|---|
| # | from | to | length | score | orientation |

| | | | | | | |
|---|---|---|---|---|---|---|
| 1 | 15 | 33 | (19) | 2066 | o-i | FLIYACLLLFSVLLALRLD |
| 2 | 40 | 56 | (17) | 1143 | i-o | YWAVFAPIWLWKLMVIV |
| 3 | 83 | 102 | (20) | 1765 | o-i | AMLIAVGIHLLLLMFEVLVC |
| 4 | 112 | 134 | (23) | 1341 | i-o | WLLVFMPLFFVSPVSVAACVWGF |
| 5 | 168 | 191 | (24) | 2978 | o-i | WLVVCVPLWILMSFLCLVVLYYIV |
| 6 | 211 | 227 | (17) | 1070 | i-o | ITMALSWMTIVVPLLTF |
| 7 | 240 | 258 | (19) | 1500 | o-i | AFSCIPIFVPLWLSLITLM |

Spacings of the segments between the TM domains are 15, 7, 27, 10, 34, 20, 13 AA, and the intracellular residue is 92 AA in length.

(b) an identical picture emerges for ee3_{—}2:

-----> STRONGLY preferred model: N-terminus outside
7 strong transmembrane helices, total score: 12351

| # | from | to | length | score | orientation |
|---|---|---|---|---|---|
| 1 | 15 | 36 | (22) | 1811 | o-i |
| 2 | 32 | 50 | (19) | 1219 | i-o |
| 3 | 83 | 102 | (20) | 1733 | o-i |
| 4 | 112 | 134 | (23) | 1330 | i-o |
| 5 | 168 | 191 | (24) | 3068 | o-i |
| 6 | 211 | 227 | (17) | 1239 | i-o |
| 7 | 243 | 260 | (18) | 1951 | o-i |

Spacings of the segments between the TM domains are 15, 0, 33, 10, 34, 20, 16 AA, and the residue is 90 AA in length.

(c) The control experiment used for comparison is the topology of the CCR-5 receptor (belongs likewise to the class of 7TM receptors) from the prior art:

| | | | | | |
|---|---|---|---|---|---|
| 1 | 51 | 76 | (26) | 2895 | o-i |
| 2 | 89 | 108 | (20) | 1155 | i-o |
| 3 | 124 | 145 | (22) | 1163 | o-i |
| 4 | 163 | 187 | (25) | 1415 | i-o |
| 5 | 220 | 239 | (20) | 2183 | o-i |
| 6 | 260 | 281 | (22) | 1782 | i-o |
| 7 | 298 | 325 | (28) | 1325 | o-i |

The spacings of the segments between the TM domains are 51, 13, 16, 18, 33, 21 and 17 AA, and the intracellular residue is 46 AA in length.

A comparison of the general topology (the number of amino acids in the respective nontransmembrane moieties of the proteins, i.e. N terminus and C terminus and the loop moieties, are shown) of the distantly related 7TM receptors bradykinin-2, CXCR5, galanine receptor-2 and anaphylotaxin C5a gives the following picture, in comparison to ee3_{—}1 of the invention:

| | N | | | | | | | |
|---|---|---|---|---|---|---|---|---|
| receptor | terminus | 1 | 2 | 3 | 4 | 5 | 6 | rest |
| C5a | 39 | 12 | 14 | 22 | 28 | 16 | 20 | 45 |
| BK-2 | 63 | 13 | 14 | 20 | 27 | 26 | 24 | 56 |
| galanin2 | 28 | 10 | 17 | 19 | 24 | 25 | 1 | 105 |
| cxcr-5 | 55 | 11 | 14 | 21 | 30 | 18 | 23 | 46 |
| mw | 43.3 | 11.7 | 15.0 | 20.3 | 26.3 | 22.3 | 15.0 | 63.0 |
| ee3_1 | 15 | 7 | 27 | 10 | 34 | 20 | 13 | 92 |

The general topology in these proteins is found to be distinctly similar.

### EXEMPLARY EMBODIMENT 7

Determination of Motifs and Signal Sequences in ee3-1

Using the Prosite Program:

Matching pattern PS00001 ASN_GLYCOSYLATION
AS 294: NISY
Total matches: 1
Matching pattern PS00006 CK2_PHOSPHO_SITE
AS 77: TCVE
Total matches: 1
Matching pattern PS00008 MYPISTYL
AS 57: GASVGT
AS 263: GQKGGN
Total matches: 2

Thus, a CK2 phosphorylation site is located in position 77, an asparagine glycosylation site in position 294 and 2 myristylation sites are located in positions 57 and 263 (continuous numbering according to FIG. 7). There is no typical phosphorylation site found in the carboxy terminus.

### EXEMPLARY EMBODIMENT 8

Induction of ee3 by Single Administration of Erythropoietin

As shown in FIG. 19, ee3_{—}1 is induced in rats at the transcriptional level by a single intraperitoneal injection of erythropoietin (Erypo, Janssen; 5000 U/kg of body weight). 6 and 24 h after injection of erythropoietin, the rat was terminally anesthetized by injecting Rompun/Ketanest, and the brain was gently removed. Control rats were treated with saline. The ee3_{—}1 messenger RNA was measured in the rat by using semiquantitative RT-PCR in the LightCycler (Roche, Mannheim, Germany). Quantification was carried out by comparing the relative fluorescence of the sample with a standard curve for cyclophilin.

Total RNA was isolated from rat forebrain (without cerebellum and olfactory bulb), using the method of Chomczynski/Sacchi (acidic phenol extraction), followed by purification using the RNeasy extraction kit according to the manufacturer's instructions (Qiagen, Santa Clarita, Calif., USA). The concentration of RNA was determined photometrically and the quality of total RNA was evaluated via agarose gel electrophoresis. The RNA was stored at −80° C. until used.

After reverse transcription with Superscript II (Invitrogen-Life Technologies, Carlsbad, Calif., USA), the reaction products were relatively quantified by real time online PCR by means of the LightCycler technology. For this purpose, total RNA samples from the brain of three wildtype mice and three transgenic tg6 mice were used. The specific oligonucleotide primer sequences for cyclophilin were

5′ACCCCACCGTGTTCTTCGAC-3′

for the forward primer and

5′CATTTGCCATGGACAAGATG-3′

for the reverse primer, with a binding temperature of 60° C., and for rat ee3_{—}1:

forward primer: 5′-GGTGTGGGAGAAATGGCTTA-3′, reverse primer: 5′-ATACCAGCAGAGCCTGGAGA-3′.

For quantification, serial cDNA dilutions of 1:3, 1:9, 1:27, 1:81 and 1:243 were amplified according to the following plan: initial denaturation at 94° C. for 5 min, amplification over 50 cycles comprising 5 s of denaturation at 94° C., 10 s of binding at 55° C. or 60° C., depending on the specific primer (see above), and 30 s of extension at 72° C. The fluorescence of each sample was measured at 80° C. at the end of each cycle for 10 s. The specificity of the reaction product was proved by means of agarose gel electrophoresis and melting curve analysis (not shown). Each PCR reaction produced exactly one reaction product.

The logarithmic phase of said PCR reaction was utilized for quantification. This involves laying an asymptote through the appropriate curve. For hemoglobin, the result was virtually parallel inclining lines so that it was possible to use the slopes of the these curves for comparison with the standard curves for cyclophilin. Averages±standard deviation were determined for each cDNA dilution of the normalized PCR product. The quantitative differences obtained in this way correspond to relative changes in RNA expression in transgenic and wildtype animals. All reactions resulted in a single reaction product. The mean induction factor for ee3 was 1.35-fold after 6 hours and 1.44-fold after 24 h.

### EXEMPLARY EMBODIMENT 9

Distribution of ee3_{—}1 RNA in the Brain

Localization of the ee3_{—}1 transcript in mice was studied by means of in-situ hybridization using a radiolabeled oligoprobe. For this purpose, brain sections of 15 μm in thickness were cut at −200 using a cryostat, mounted on poly-L-lysine-coated slides and fixed in 4% paraformaldehyde in PBS (pH 7.4). The oligonucleotide was radiolabeled with a-³⁵S-dATP by means of terminal tranferase (Roche Diagnostics, Mannheim). Labeling as well as subsequent hybridization were carried out according to a protocol by Wisden & Morris (In situ-Hybridization Protocols for the brain, Academic Press 1994).

The radiolabeled probe used (ee3_{—}1.3 as AACGAAGGGCCAGTAGCACAGAGAACAGCAGCAGACAGGCATAGATGAGG) was able to make visible ee3_{—}1 expression in the cerebellum (ce), hippocampus (hc), dentate gyrus (dg) and in the cortex (co), in particular in the entorhinal cortex (ent), in the olfactory bulb (olf). A corresponding sense control (ee3_{—}1.3s, CCTCATCTATGCCTGTCTGCTGCTGTTCTCTGTGCTACTGGCCCTTCGTT) gave no specific signal (not shown) (FIG. 20).

### EXEMPLARY EMBODIMENT 10

Immunohistochemical Representation of ee3_{—}1 Distribution in Mouse Tissue

Paraffin-embedded tissue was cut (2 μm), mounted on pretreated slides (DAKO, Glostrup, Denmark), dried in air overnight and subsequently deparaffined (xylene and descending order of alcohols). After microwave treatment in citrate buffer at 500 W for 10 min, the sections were incubated with anti-ee3_{—}1 serum (AS4163) in a dilution of 1:500 in a humid chamber at room temperature for 1 h. The immunoreaction was made visible by ABC technology using DAB as a chromogen, according to the manufacturer's information (DAKO, Glostrup, Denmark). Negative controls comprised equally treated sections, but with the primary antibody being omitted, and also sections for which, instead of the primary antibody, the corresponding pre-immune serum was used.

The results of the immunohistochemical stainings are depicted in FIG. 22. Over all, a substantially neuron-specific localization of ee3 is revealed, with the exception of some structures in the intestine and in the lung. ee3 is frequently expressed by neurons having integrative functions (the large pyramidal cells of the cortical layer V, mitral cells in the olfactory bulb, Purkinje cells in the cerebellum). The images A-C show cortical localizations of ee3 in layer V, with distinct staining of neuronal projections. A more intensive immunostaining is visible in the entorhinal cortex (C). From a physiological point of view, information flows from the entorhinal cortex to the hippocampus, contributing to learning and memory.

Alterations of the entorhinal cortex are frequently found in patients with stroke, Alzheimer's disease or after head and brain injury. Disorders of the entorhinal cortex may cause changes in behavior, which include insufficient processing of sensorial impressions and learning difficulties (Davis et al., Nurs Res 50 (2) 77-85 (2001)). The images D-F show the hippocampal distribution pattern of ee3. The sharp boundary between expression in the CA3 sector and the lack of expression in the CA2 and CA1 sectors is conspicuous here. Neurons of the CA1 region and, to a lesser extent, also of the CA4 region are particularly susceptible to the necrosis- and inflammation-free physiological cell death (apoptosis), in particular with existing general central-nervous damage (e.g. (Hara, et al., Stroke, 31, 236-8, (2000)). In contrast, the dentate gyrus seems to be affected rather by necrotic damage. The dentate gyrus is linked to de novo neuron formation following pathological stimuli (Takagi, et al., Brain Res, 831, 283-7, (1999)) (Parent, et al., J Neurosci, 17, 3727-38, (1997)). ee3 is likewise found in areas of non-neocortical genesis: in the Purkinje cells of the cerebellum (G, H), which act there as integrating neurons, and in the mitral cells of the olfactory bulb (I, J). Intensive expression of ee3 can be found in the ganglial cells and in the sensory cells of the retina (K, L).

Recently, the neuroprotective action of erythropoietin in the retina was reported (Junk et al., Erythropoietin administration protects retinal neurons from acute ischemia-reperfusion injury. Proc Natl Acad Sci USA. 2002 Aug. 6; 99(16):10659-64.; Grimm et al., HIF-1-induced erythropoietin in the hypoxic retina protects against light-induced retinal degeneration. Nat. Med. 2002 July; 8(7):718-24.) These findings suggest a connection between EPO induction and ee3 expression. EE3 is likewise strongly expressed in neurons belonging to the motor system. Thus, specific expression can be found in the spinal cord in the large motoneurons of the anterior horn (FIG. 22 M) and in the functionally equivalent neurons of the motor nucleus of the trigeminal nucleus (FIG. 22 N).

Said distribution of ee3 in the spinal cord may possibly be utilized for therapeutic and diagnostic intervention in amyotrophic lateral sclerosis. Amyotrophic lateral sclerosis (ALS; Lou Gehrig's disease; Charcot's disease) is a neurodegenerative disorder with an annual incidence of from 0.4 to 1.76 per 100 000 (Adams et al., Principles of neurology, 6th ed., New York, pp 1090-1095). It is the most common form of motoneuron disorders with typical manifestations such as generalized fasciculations, progressive atrophy and weakness of the skeletal muscular system, spasticity and positive pyramidal tract signs, dysarthria, dysphagia, and dyspnea. The pathology mainly comprises the loss of nerve cells in the anterior horn of the spinal cord and in the motor nuclei of the lower brain stem, but may also affect the first order motoneurons in the cortex. The pathogenesis of this disease is largely unknown, although the role of superoxide dismutase mutations in familial cases has been explained very well. To date, more than 90 mutations in the SOD1 protein, which may cause ALS, have been described (Cleveland and Rothstein (2001), Nat Rev Neurosci, 2, 806-19.). Neurofilaments also seem to play a part in this disease. Excitotoxicity, a mechanism triggered by an excess of glutamate, is another pathogenetic factor, and this can be confirmed by the action of riluzole in human patients. Activation of caspases and apoptosis together seem to be the final route of ALS pathogenesis (Ishigaki, et al. (2002), J Neurochem, 82, 576-84., Li, et al. (2000), Science, 288, 335-9.). Localization of the ee3 protein on the neurons affected in ALS clearly indicates the potential therapeutically functional/diagnostic applicability of ee3 agonists or ee3 antagonists in this disease.

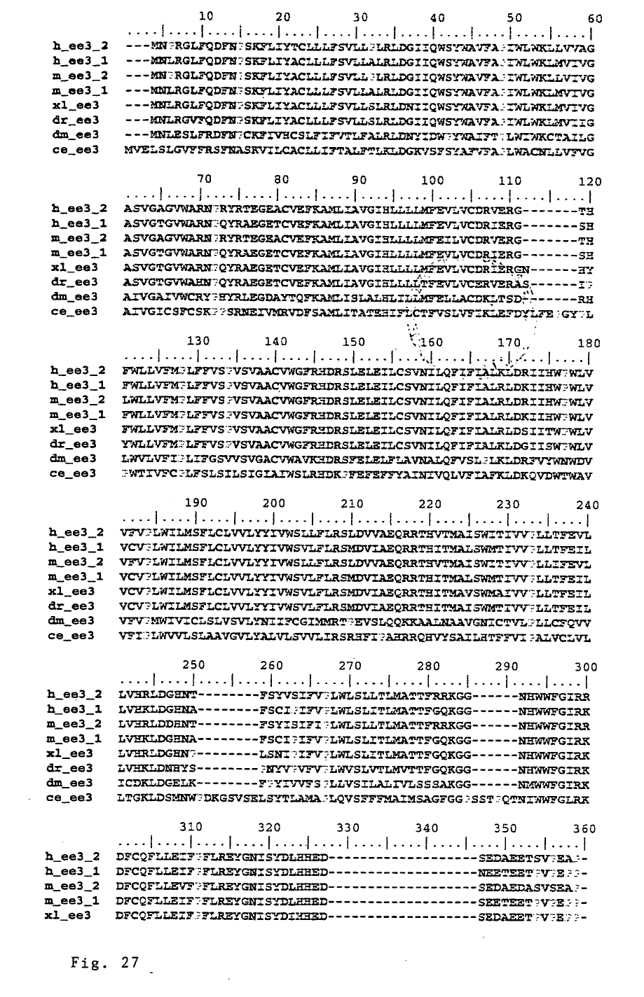

The localization of ee3 in the substantia nigra of the midbrain (FIG. 220, P) may open up therapeutic and diagnostic possibilities for Parkinson's disease. Parkinson's disease is the most common movement disorder with approximately 1 million patients in North America. Approx. 11 of the over 65 population is affected. The major symptoms are rigor, tremor, akinesia (Adams et al., Principles of neurology, 6th ed., New York, pp 1090-1095). The cause of the disease is unknown. Nevertheless, analyses of post-mortem tissue and of animal models indicate a progressive process of oxidative stress in the substantia nigra, which could sustain dopaminergic neurodegeneration. Oxidative stress which may be caused by neurotoxins such as 6-hydroxydopamine and MPTP (N-methyl-4-phenyl-1,2,3,6-tetrahydropyridine) is used in animal models in order to study the process of neurodegeneration. Although a symptomatic therapy exists (e.g. L-DOPA plus a decarboxylase inhibitor; bromocriptine, pergolide as dopamine agonists and anticholinergic substances such as trihexyphenidyl (artane)), there is a clear need for a causal, i.e. neuroprotective, therapy which can stop the pathological process. Apoptotic mechanisms are clearly involved in the pathogenesis, both in the animal model and in humans (Mochizuki, et al. (2001), Proc. Natl. Acad. Sci. USA, 98, 10918-23, Xu et al. (2002), Nat. Med., 8, 600-6, Viswanath, et al. (2001), J. Neurosci., 21, 9519-28, Hartmann, et al. (2002), Neurology, 58, 308-10).

Localization of ee3 in the nervous system is very strong evidence for a connection between expression of this protein and neuronal cell death, neurogenesis and neural plasticity.

In the lung, ee3 can be found in distinct structures (FIG. 22 Q-V). Basal cells expressing ee3 are found in the terminal bronchioli and are possibly cells having neuroendocrine activity. This localization suggests a therapeutical importance of ee3 for diseases of the bronchi. There is also expression in the endothelia and smooth muscle cells of arterioles (12 U, V). In contrast, venoles do not exhibit any immunohistochemically recordable expression of ee3 (FIGS. 22 Q and R). Expression of particular receptors by endothelial cells is of great pharmacological importance, since therapeutics immediately contact this cell layer in the blood. Important drugs acting on the circulation target the arterioles, in particular the endothelium or the smooth muscular system. ee3 is therefore a very attractive target protein for influencing circulatory disorders, for example arterial hypertension.

In the intestine, ee3 is found basally in the crypts (FIG. 22 W, X) and in nerve cells which presumably belong to the enteric plexus (FIG. 22 Y). In most of the histologically studied organs, there is no specific staining of organ-specific cells, but rather in many cases only a distinct staining of nerves (see, for example, in the heart muscle, FIG. 22 AA, or in the connective tissue, FIG. 22 Z). This clear localization of ee3 on axons predisposes the molecule to diagnosis and therapy of disorders of the peripheral nerves (neuropathies), which include, for example, the widespread diabetic polyneuropathy. Likewise, common genetic disorders of the peripheral nervous system, for example the HMSN group (hereditary motorsensory neuropathies), could also profit therefrom.

Finally, an expression pattern was found in skeletal muscle, which is most likely consistent with the localization on motor end plates (FIG. 22 BB). This is potentially interesting for disorders of the motor end plate, for example myasthenia gravis.

### EXEMPLARY EMBODIMENT 11

ee3 is Upregulated at the Protein Level in EPO-Overexpressing Mice

Immunohistochemistry (antiserum AS 4163) also showed distinct upregulation of the ee3 protein by erythropoietin (FIG. 23). CNS sections treated in parallel have distinctly enhanced signals for ee3. This was shown on in each case 3 mice in total (wild type and EPO-overexpressing (tg6)) which were stained and assessed in each case in a blinded study with respect to the genotype.

### EXEMPLARY EMBODIMENT 12

Colocalization of ee3 and map1b and Absence of ee3 Expression in map1b-Deficient Mice

The light chains of Map1a and Map1b were identified as interacting proteins in a yeast two-hybrid screening with the ee3_{—}1 carboxy terminus (see previous examples, exemplary embodiment 4). FIG. 24 depicts a double immunofluorescence for ee3_{—}1 and map1b in mice, and demonstrates the unexpected overlapping of ee3_{—}1 and map1b expression in mice.

For double immunofluorescence stainings, deparaffined sections were incubated, after microwave treatment (citrate puffer, 500 W, 10 min), simultaneously with the rabbit ee3_{—}1 antibody (AS4163) and a goat antibody directed against map 1a (MAP-1B (C20): sc-8971; Santa Cruz; Santa Cruz, USA) in a humid chamber at room temperature for one hour. After appropriate washing steps, the sections were incubated with a mixture of the secondary antibodies FITC anti-rabbit and TRITC anti-goat for 30 min (both antibodies diluted in each case 1:30 in PBS, obtained from Dianova, Hamburg, Germany). After the sections had been washed again with PBS, the preparations were sealed in using Histosafe and analyzed in a fluorescence microscope (Olympus IX81, Olympus, Germany) using appropriate barrier filters. Signal overlays were prepared with the aid of Analysis software (soft imaging systems, Stuttgart, Germany). In parallel single fluorescence stainings, in each case the absence of a signal in the other channel was demonstrated, ruling out the phenomenon of signals “emitting into” the in each case other channel, for example due to insufficient filters. Double staining with interchanged chromophores for the secondary antibody gives the same picture (not shown).

FIG. 24 depicts an astonishing co-localization of the two proteins in the CNS. Green: ee3_{—}1 staining; red: Map1b staining; yellow: electronic superimposition of both signals. Examples from the spinal cord (sc) and from the cerebellum (cb) are shown.

Map1b is an important neuronal protein. It is one of the first microtubule associated proteins (maps) which are expressed during development of the mammalian central nervous system. An involvement in axonogenesis in neurons is likely (Gonzalez-Billault, et al. (2001), Mol Biol Cell, 12, 2087-98, Gonzalez-Billault, et al. (2002), Brain Res, 943, 56-67.). A functionally important part of map1b in the interaction of neurons is supported by very recent studies which demonstrate that map1b is involved in the pathogenesis of fragile X syndrome which is the most common hereditary form of mental retardation. map1b mRNA is controlled by FMRP, a protein directly regulated by fragile X mRNA. A study in Drosophila found that map1b (futsch in Drosophila) plays a central part in the manifestation of the fragile X-analogous phenotype (Sohn (2001), Science, 294, 1809, Zhang, et al. (2001), Cell, 107, 591-603.). map1b also binds to gigaxonin, a protein whose mutated gene is responsible for the recessive genetic disease giant axonal neuropathy (GAN) (Ding, et al. (2002), J Cell Bol, 158, 427-33.). After lesion of peripheral nerves, map1b is increasingly induced in the outgrowing neurons of myelinated nerves and is probably involved in axonal sprouting (Soares, et al. (2002), Eur J Neurosci, 16, 593-606.). Finally, map1b probably localizes the GABA-C receptor to its synaptic location (Pattnaik, et al. (2000), J Neurosci, 20, 6789-96.).

The map1b gene was genetically inactivated in mice (knockout). The best available knockout is the mouse described in Meixner et al. (Meixner, Haverkamp, Wassle, Fuhrer, Thalhammer, Kropf, Bittner, Lassmann, Wiche and Propst (2000), J Cell Biol, 151, 1169-78.). Mice with homozygous inactivation of the map1b gene were studied immunohistochemically for ee3 expression. The stainability of ee3_{—}1 (FIG. 25) was found to be very greatly reduced, indicating a dependence of ee3_{—}1 protein expression or protein stability on the interaction with map1b. An inhibitor of this interaction would thus lead to markedly reduced ee3 expression.

### EXEMPLARY EMBODIMENT 13

Preparation of an Antiserum for Detecting ee3_{—}1

The protein sequence of human ee3_{—}1 protein was analyzed using the Protean program part of the DNAStar program package (Lasergene) and the epitope LHHEDNEETEETPVPEP corresponding to amino acids 299-315 and located in the intracellular C terminus was selected according to secondary prediction and also to high predicted surface probability and high antigenicity. Said peptide sequence was synthesized with cysteine attached to the N terminus (CLHHEDNEETEETPVPEP) in order to make possible a controlled specific coupling to the carrier protein KLH (keyhole limpet hemocyanine). Two rabbits were immunized with the peptide-KLH conjugate according to an optimized plan. Peptide synthesis, subsequent coupling to KLH and immunization of two rabbits were ordered from BioTrend Chemikalien GmbH. The pre-immune serum of several rabbits was assayed, prior to the primary immunization, for its crossreactivity in Western blot analyses of mock-transfected cells and brain extracts. Two rabbits with negligible background received the first boost 3 weeks after the primary immunization and the second boost another 4 weeks later. One week later, 20 ml of blood were taken from the rabbits and the sera were assayed in Western blot analyses and immunocytochemical stainings of transiently transfected cells (HEK293, CHO-dhfr−). After the 3rd or 4th boost, the rabbits were bled.

The sera of both rabbits were positive. In particular, the AS4163 antiserum recognized in both methods very specifically transiently expressed human ee3_{—}1 protein in various cells and was able to be used in Western blot analyses up to a dilution of 1:12 000. The AS4163 antiserum is also suitable for immunoprecipitation of ee3_{—}1 protein and may therefore be used for precipitation of ee3_{—}1 and proteins interacting therewith from transfected cells or native tissue. The AS4163 antiserum recognizes in particular the corresponding epitope in the murine ee3_{—}1 sequence which differs from the human sequence only by one amino acid, namely the mutation of N3O4 to serine. The AS4163 antiserum is therefore very well suited to immunohistochemical analysis of ee3_{—}1 protein expression in wild type, transgenic and knockout mice, as FIGS. 22-24 show.

### EXEMPLARY EMBODIMENT 14

ee3_{—}1 is Expressed by Neural Stem Cells

Neural stem cells were isolated from the hippocampus of 4-6 week old male Wistar rats, as previously described (Ray et al., 1993). The protocols are consistent with German law. The animals were anesthetized with 1% (v/v) isoflurane, 70% N₂O, 29% oxygen and sacrificed by decapitation. The brains were prepared and washed in 50 ml of ice-cold Dulbecco's phosphate buffered saline (DPBS) containing 4.5 g/l glucose (DPBS/Glc). The hippocampus was prepared out of 6 animals, washed in 10 ml DPBS/Glc and centrifuged at 1600×g at 4° C. for 5 min. After removing the supernatant, the tissue was homogenized using scissors and a scalpel. The tissue pieces were washed with DPBS/Glc medium, centrifuged at 800 g for 5 min, and the pellet was resuspended in 0.01% (w/v) papain, 0.1% (w/v) dipase II (neutral protease), 0.01% (w/v) DNase I, and 12.4 mM manganese sulfate in Hank's balanced salt solution (HBSS). The tissue was triturated using pipette tips and incubated at room temperature for 40 min, with occasional mixing of the solution (every 10 min). The suspension was then centrifuged at 800×g and 4° C. for 5 min, and the pellet was washed three times in 10 ml of DMEM Ham's F-12 medium containing 2 mM L-glutamine, 100 units/ml penicillin and 100 units/ml streptomycin. The cells were then resuspended in 1 ml of neurobasal medium containing B27 (Invitrogen, Carlsbad, Calif., USA), 2 mM L-glutamine, 100 units/ml penicillin and 100 units/ml streptomycin, 20 ng/ml EGF, 20 ng/ml FGF-2, and 2 μg/ml heparin. The cells were seeded under sterile conditions into 6-well plates at a concentration of 25 000-100 000 cells/ml. The plates were incubated at 37° C. in 5% CO₂. The cell culture medium was changed once a week, replacing approximately only ⅔ of the medium. (ref: Ray J, Peterson D A, Schinstine M, Gage F H (1993) Proliferation, differentiation, and long-term culture of primary hippocampal neurons. Proc Natl Acad Sci USA 90: 3602-6.).

RNA was isolated according to standard protocols (RNeasy kit, Qiagen) from hippocampal stem cells which had been cultured for 3 weeks, after they had been thawed from frozen stocks. cDNA was synthesized according to standard protocols using oligodT primers and Superscript II reverse transcriptase (Gibco). A PCR was carried out using the following reaction parameters: denaturation 94° C. for 10 min, 30 cycles at 94° C. for 30 s, 55° C. for 50 s, 72° C. for 60 s; 72° C. for 5 min, 4° C., using the following primer pairs: ee3_plus 5′-GGTGTGGGAGAAATGGCTTA-3′ and ee3_minus 5′-ATACCAGCAGAGCCTGGAGA-3′.

In recent years, the importance of the novel formation of nerve cells (neurogenesis) in the course of neurological diseases has been recognized. In contrast to many other tissues, the mature brain has limited regenerative capacities, and the unusually high degree of cellular specialization limits the possibilities for remaining healthy tissue to take over the function of the destroyed tissue. Nerve cells developing from precursor cells in the adult brain, however, have the potential in principle to take over those functions.

Neurogenesis occurs in discrete regions of the adult brain (the rostral subventricular zone (SVZ) of the lateral ventricles and the subgranular zone (SGZ) in the dentate gyrus (DG). Many groups have demonstrated that neurogenesis is induced in particular by neurological damage (e.g. cerebral ischemia (Jin, et al. (2001), Proc. Natl. Acad. Sci. USA, 98, 4710-5, Jiang, et al. (2001), Stroke, 32, 1201-7, Kee, et al. (2001), Exp. Brain. Res., 136, 313-20, Perfilieva, et al. (2001), J. Cereb. Blood Flow Metab., 21, 211-7)). Neurogenesis also occurs in humans (Eriksson, et al. (1998), Nat Med, 4, 1313-7.), and indeed leads to functional neurons (van Praag, et al. (2002), Nature, 415, 1030-4). The subgranular zone of the dentate gyrus and the hilus have the potential of generating new neurons during adult life (Gage, et al. (1998), J Neurobiol, 36, 249-66). Conspicuously, ee3 can be detected on neuronal stem cells of the hippocampus (FIG. 25). This implies great importance of ee3 for neurogenesis. This importance in neurogenesis is further evidence for the usefulness of ee3 for any neurodegenerative disorders in general.

In contrast to the action on endogenous stem cells in the brain, therapeutics interfering with ee3 for in vitro manipulation of stem cells (e.g. in vitro differentiation and proliferation). Currently, stem cells are explored for their usability in a number of neurodegenerative disorders, in particular Parkinson's disease and stroke. It is desirable, for example, to differentiate cells in vitro for transplantation for Parkinson patients and thus to compensate for the dopaminergic deficit after injection (replacement therapy) (Arenas (2002), Brain Res. Bull, 57, 795-808, Barker (2002), Mov. Disord., 17, 233-41). Another possibility of introducing stem cells are, for example, intraarterial or intravenous injections in the case of stroke or brain injury (Mahmood, et al. (2001), Neurosurgery, 49, 1196-203; discussion 1203-4, Lu, et al. (2001), J Neurotrauma, 18, 813-9, Lu, et al. (2002), Cell Transplant, 11, 275-81, Li et al. (2002), Neurology, 59, 514-23). Another possible use of ee3 in stem cell therapy would be the preparation of cells which constantly secrete an agonist or antagonist for ee3.

### EXEMPLARY EMBODIMENT 15

Cloning of Additional Relatives of the ee3 Receptor Family from Xenopus laevis and Dario rerio

It was possible to clone additional members of the ee3 protein family owing to the homology criteria of the invention. EST databases were screened with protein sequences from the human ee3_{—}1 protein, using TBLASTN, resulting in ESTs from X. laevis (African clawed frog) and from D. rerio (zebra fish). Said ESTs were sequenced using standard methods, resulting in the following sequences:

Full-length sequence of x1_ee3 (Xaenopus laevis):

CCCGGGCACGTTACCGTATTGATGTTACTAGTAGCGCACAGAAACATCCT
GGTCTAAGCAGTTGCAGCAGGTACTGCGTTGTAGTGGCGGTAGTTACGAC
TCTGTAGGTTAGAGCGGAGGCTTTGCTGGAGCAATGTCCGCCTAGTGAAG
CTCGGAGAGGTGCTCGCACCATGAATCTTAGGGGCCTCTTCCAGGATTTT
AACCCCAGTAAATTTCTCATCTACGCATGTTTGTTGCTCTTTTCTGTTCT
CCTTTCCCTGCGACTGGATAATATTATTCAGTGGAGTTACTGGGCGGTGT
TTGCTCCAATATGGTTGTGGAAACTAATGGTTATTGTGGGAGCCTCAGTT
GGTACAGGTGTATGGGCACGTAACCCTCAATACAGGGCAGAAGGTGAAAC
ATGTGTGGAGTTCAAGGCCATGCTAATTGCAGTGGGAATTCATTTGCTGC
TTCTTATGTTTGAAGTTCTTGTTTGCGATCGTATTGAAAGAGGAAACCAC
TACTTCTGGTTGCTAGTCTTTATGCCTTTATTCTTTGTGTCCCCAGTATC
CGTTGCAGCTTGCGTTTGGGGCTTTCGGCATGATCGATCATTGGAATTGG
AAATCTTGTGCTCCGTCAATATTCTGCAGTTTATATTCATTGCCCTAAGA
CTTGACAGCATCATCACTTGGCCTTGGCTTGTGGTATGTGTCCCGCTGTG
GATCCTTATGTCCTTCCTGTGCCTAGTAGTTCTGTATTATATTGTGTGGT
CAGTTCTGTTCCTGCGTTCAATGGATGTTATTGCAGAACAAAGGAGAACT
CATATTACTATGGCAGTCAGTTGGATGGCTATAGTTGTACCGCTTCTGAC
ATTCGAGATATTACTTGTTCATCGACTTGATGGGCACAATCCATTATCGA
ATATCCCTATATTTGTTCCGCTTTGGCTTTCCTTAATAACGTTGATGGCA
ACAACCTTTGGACAGAAAGGAGGCAATCACTGGTGGTTTGGGATTCGTAA
AGACTTCTGCCAGTTTCTGTTGGAGATTTTCCCTTTTCTTCGAGAATATG
GCAATATCTCATATGATATTCATCATGAAGACAGTGAAGATGCTGAAGAA
ACACCTGTACCGGAGCCCCCCAAAATCGCACCAATGTTTCGAAAGAAGAC
TGGCGTTGTCATTACCCAGAGCCCAGGGAAATATATTGTTCCTCCTGCTA
AACTTAACATCGACATGCCGGATTAAGGTGAAATTTGGTGGCTTGAGGGC
ACTTTTTTCTGTTTTAACTAATCCTGTTAGTAGTACACTATCAGGTGTCA
TGGACTGAAGGGAAAAAAAGACTACTGACCTCATTCCTTTTTTGTATTCA
TTTGTAATTTTTTTTGTTCCTGCAATGGTATGTGTTTTCCCATTCCTAAT
TCATGTCATCATGTTACTCAAGATCAGGGAAGCTTCTTAAGGGCAAAGAA
TGCTGGAATTTGTAGTTTATAATTTGTGGATGACTATAAATTTTCACATC
TGTTGTCTTGGTAATGACTGCAGTCTTGCATTCTAGTTTCTAGTAACACA
GAGATAGACCAGCTGTGGCCCTCCAGATACTGAGCTAACAAGCTTTGGGA
GACATCCTGGGAATCTTAGCAGCTCTGGGGCCACAGGTTGGACTTCTCAG
CAGTAAAATTAAGTATAATGTTTATCTTAAGTAAATGTCTTTGTGTGTGT
TGTTATGCAATGCAGCTATTGTTTGATATCTTTAcagcagaacttgtgca
tagaattgaattcaagttgtgagctgttttataccactataaaaatactt
ttAAAAAAAAATCTGTTTAAGGGTCAAGCATTACCTTGGAGAAGTGATAT
TTGAGCAGAGGGCTTATGGGATATATCTAATATACACCTTCCCTTAGGAG
TTACTACTCCTTGCTCACTTGTATAGTATTTATAAGAACATTTTATCAAT
GTAATATATTGTGTTCAAAATTATTCTTATGTACAGTATAAATGGATAAA
TACAAAGTATTTTTTTAAATAAAAGATGTAAAATACATATAAGTTGTCAA
AATTTTGTTTGTAATTTACATTTTAAAATGATCTATGTGAATTCTACAAT
GAAAAAAGATCTATACAATTTCAAAAGCCAGTATGTCATTTTTATATACT
GACCATGTACATATTATGTAAGATGTAAAGCCAAACACCAATGACATGAA
TGTTAAGTTATTAGACTATGAATAAAACATTGATTTTATTTTATGTTGTA
AAAAAAAAAAAAAAAAA

The open reading frame of x1_ee3:

ATGAATCTTAGGGGCCTCTTCCAGGATTTTAACCCCAGTAAATTTCTCAT
CTACGCATGTTTGTTGCTCTTTTCTGTTCTCCTTTCCCTGCGACTGGATA
ATATTATTCAGTGGAGTTACTGGGCGGTGTTTGCTCCAATATGGTTGTGG
AAACTAATGGTTATTGTGGGAGCCTCAGTTGGTACAGGTGTATGGGCACG
TAACCCTCAATACAGGGCAGAAGGTGAAACATGTGTGGAGTTCAAGGCCA
TGCTAATTGCAGTGGGAATTCATTTGCTGCTTCTTATGTTTGAAGTTCTT
GTTTGCGATCGTATTGAAAGAGGAAACCACTACTTCTGGTTGCTAGTCTT
TATGCCTTTATTCTTTGTGTCCCCAGTATCCGTTGCAGCTTGCGTTTGGG
GCTTTCGGCATGATCGATCATTGGAATTGGAAATCTTGTGCTCCGTCAAT
ATTCTGCAGTTTATATTCATTGCCCTAAGACTTGACAGCATCATCACTTG
GCCTTGGCTTGTGGTATGTGTCCCGCTGTGGATCCTTATGTCCTTCCTGT
GCCTAGTAGTTCTGTATTATATTGTGTGGTCAGTTCTGTTCCTGCGTTCA
ATGGATGTTATTGCAGAACAAAGGAGAACTCATATTACTATGGCAGTCAG
TTGGATGGCTATAGTTGTACCGCTTCTGACATTCGAGATATTACTTGTTC
ATCGACTTGATGGGCACAATCCATTATCGAATATCCCTATATTTGTTCCG
CTTTGGCTTTCCTTAATAACGTTGATGGCAACAACCTTTGGACAGAAAGG
AGGCAATCACTGGTGGTTTGGGATTCGTAAAGACTTCTGCCAGTTTCTGT
TGGAGATTTTCCCTTTTCTTCGAGAATATGGCAATATCTCATATGATATT
CATCATGAAGACAGTGAAGATGCTGAAGAAACACCTGTACCGGAGCCCCC
CAAAATCGCACCAATGTTTCGAAAGAAGACTGGCGTTGTCATTACCCAGA
GCCCAGGGAAATATATTGTTCCTCCTGCTAAACTTAACATCGACATGCCG
GATTAA

and the protein sequence of x1_ee3:

MNLRGLFQDFNPSKFLIYACLLLFSVLLSLRLDNIIQWSYWAVFAPIWLW
KLMVIVGASVGTGVWARNPQYRAEGETCVEFKAMLIAVGIHLLLLMFEVL
VCDRIERGNHYFWLLVFMPLFFVSPVSVAACVWGFRHDRSLELEILCSVN
ILQFIFIALRLDSIITWPWLVVCVPLWILMSFLCLVVLYYIVWSVLFLRS
MDVIAEQRRTHITMAVSWMAIVVPLLTFEILLVHRLDGHNPLSNIPIFVP
LWLSLITLMATTFGQKGGNHWWFGIRKDFCQFLLEIFPFLREYGNISYDI
HHEDSEDAEETPVPEPPKIAPMFRKKTGVVITQSPGKYIVPPAKLNIDMP
D

For D. rerio, these sequences are as follows (dree3):

CTCGAGCACTGTTGGCCTACTGGGATGTGAGTGCCAGTCAGCTAGCCAGC
CTCTCCTTTTCAGTTCATGTAACTATGGTCTGAAGAGGAAACCATGAATC
TCCGAGGCGTTTTCCAAGATTTCAACCCCAGTAAGTTCCTGATCTACGCA
TGTCTGCTGCTCTTCTCTGTGCTGCTGTCACTGAGGCTGGATGGCATCAT
CCAGTGGAGCTACTGGGCCGTGTTTGCGCCCATCTGGCTCTGGAAGCTCA
TGGTCATCATCGGGGCGTCTGTGGGCACTGGAGTGTGGGCTCACAACCCG
CAGTACAGGGCTGAAGGGGAGACGTGTGTGGAGTTTAAGGCCATGCTGAT
CGCAGTGGGAATCCACCTGCTCCTGCTCACCTTCGAGGTGCTGGTCTGCG
AGCGCGTGGAACGGGCTTCGATCCCCTACTGGCTCCTGGTGTTCATGCCG
CTCTTCTTCGTCTCTCCGGTGTCAGTGGCAGCGTGTGTGTGGGGATTCAG
ACACGACCGCTCGCTGGAGCTGGAGATTCTGTGCTCTGTAAATATTCTTC
AGTTTATCTTCATCGCTCTGAAACTGGACGGGATCATCAGCTGGCCGTGG
CTGGTGGTGTGTGTGCCGCTCTGGATCCTCATGTCCTTCTTGTGTCTGGT
GGTcctctattatatcgtgtggtctgTGCTGTTTCTGCGCTCCATGGATG
TGATCGCGGAGCAGCGGCGCACACACATCACCATGGCCATCAGCTGGATG
ACTATAGTCGTGCCCCTGCTCACTTTTGAGATTCTCCTCGTCCACAAGCT
gGATAATCATTATAGCCCCAACTACGTcCCGGTGTTTGTTCCTCTCTGGG
TTTCTTTAGTGACTCTAATGGTGACCACATTTGGCCAGAAAGGAGGCAAT
CACTGGTGGTTTGGCATCCGTAAAGACTTCTGCCAGTTTCTgCTGGAGCT
CTTCCCGTTCCTCAGGGAATATGGCAACATCTACTATGACCTGCATcACG
AGGACTCAGACATGTcCGAGGAGTTGCCCATTCACGAGGTGCCCAAAATC
CCTACCATGTTTAgCAAGAAGACGGGGGTGGTGATCACCCAAAGCCCTGG
GAAATACTTTGTGCCCCCACCCAAACTGTGCATCGACATGCCAGACTAAC
ATTGGAGCTCTCGTATACAGTATAGCACTATGCAATGGAATTCGCTTTGT
TACGTgCTGTTGAAGACGGcAACAACAATCCCATTAAACTCGGCTCTTGT
TTCCTAAAAAAAATAGCTGCGCAAACGGACCTGTTGACATCA

The open reading frame of dr_ee3:

ATGAATCTCCGAGGCGTTTTCCAAGATTTCAACCCCAGTAAGTTCCTGAT
CTACGCATGTCTGCTGCTCTTCTCTGTGCTGCTGTCACTGAGGCTGGATG
GCATCATCCAGTGGAGCTACTGGGCCGTGTTTGCGCCCATCTGGCTCTGG
AAGCTCATGGTCATCATCGGGGCGTCTGTGGGCACTGGAGTGTGGGCTCA
CAACCCGCAGTACAGGGCTGAAGGGGAGACGTGTGTGGAGTTTAAGGCCA
TGCTGATCGCAGTGGGAATCCACCTGCTCCTGCTCACCTTCGAGGTGCTG
GTCTGCGAGCGCGTGGAACGGGCTTCGATCCCCTACTGGCTCCTGGTGTT
CATGCCGCTCTTCTTCGTCTGTCCGGTGTCAGTGGCAGCGTGTGTGTGGG
GATTCAGACACGACCGCTCGCTGGAGCTGGAGATTCTGTGCTCTGTAAAT
ATTCTTCAGTTTATCTTCATCGCTCTGAAACTGGACGGGATCATCAGCTG
GCCGTGGCTGGTGGTGTGTGTGCCGCTCTGGATCCTCATGTCCTTCTTGT
GTCTGGTGGTcctctattatatcgtgtggtctgTGCTGTTTCTGCGCTCC
ATGGATGTGATCGCGGAGCAGCGGCGCACACACATCACCATGGCCATCAG
CTGGATGACTATAGTCGTGCCCCTGCTCACTTTTGAGATTCTCCTCGTCC
ACAAGCTgGATAATCATTATAGCCCCAACTACGTcCCGGTGTTTGTTCCT
CTCTGGGTTTCTTTAGTGACTCTAATGGTGACCACATTTGGCCAGAAAGG
AGGCAATCACTGGTGGTTTGGCATCCGTAAAGACTTCTGCCAGTTTCTgC
TGGAGCTCTTCCCGTTCCTCAGGGAATATGGCAACATCTACTATGACCTG
CATcACGAGGACTCAGACATGTcCGAGGAGTTGCCCATTCACGAGGTGCC
CAAAATCCCTACCATGTTTAgCAAGAAGACGGGGGTGGTGATCACCCAAA
GCCCTGGGAAATACTTTGTGCCCCCACCCAAACTGTGCATCGACATGCCA
GACTAA

and the protein sequence of dr_ee3:

MNLRGVFQDFNPSKFLIYACLLLFSVLLSLRLDGIIQWSYWAVFAPIWLW
KLMVIIGASVGTGVWAHNPQYRAEGETCVEFKAMLIAVGIHLLLLTFEVL
VCERVERASIPYWLLVFMPLFFVSPVSVAACVWGFRHDRSLELEILCSVN
ILQFIFIALKLDGIISWPWLVVCVPLWILMSFLCLVVLYYIVWSVLFLRS
MDVIAEQRRTHITMAISWMTIVVPLLTFEILLVHKLDNHYSPNYVPVFVP
LWVSLVTLMVTTFGQKGGNHWWFGIRKDFCQFLLELFPFLREYGNIYYDL
HHEDSDMSEELPIHEVPKIPTMFSKKTGVVITQSPGKYFVPPPKLCIDMP
D

FIG. 27 once more illustrates the high evolutionary conservation of the ee3 family, with the aid of the two proteins from X. laevis and D. rerio.

1-27. (canceled)

28. A DNA sequence, which codes for a polypeptide according to any of FIGS. 13, 14, 15A, 15B, 15C, 16 or 18, including any functionally homologous derivatives thereof.

29. A DNA sequence as claimed in claim 28, which comprises a (c) DNA sequence according to any of FIGS. 9A, 10, 11A, 11B, 11C, 12 and 17 for the translated region (in capital letters).

30. An expression vector, which comprises a DNA sequence as claimed in claim 28.

31. A host cell, which is transformed with an expression vector as claimed in claim 30.

32. A host cell as claimed in claim 31, which is a mammalian cell.

33. A host cell as claimed in claim 32 which is a human cell.

34. A purified gene product, which is encoded by a DNA sequence as claimed in claim 28.

35. A purified gene product as claimed in claim 34, which is a polypeptide.

36. A transgenic animal, which lacks at least one native ee3 amino acid sequence according to any of FIGS. 13, 14, 15A, 15B, 15C, 16 and 18, or parts thereof.

37. An antibody, which recognizes an epitope on a gene product as claimed in claim 34.

38. An antibody as claimed in claim 37, which is monoclonal.

39. An antibody as claimed in claim 37, which is directed against a sequence section on the extracellular domain as epitope.

40. A method for expressing gene products as claimed in claim 34, wherein host cells are transformed with an expression vector comprising a DNA sequence encoding a polypeptide according to any of FIGS. 13, 14, 15A, 15B, 15C, 16 or 18.

41. A method for isolating gene products as claimed in claim 34; wherein host cells are transformed with an expression vector comprising a DNA sequence encoding a polypeptide according to any of FIGS. 13, 14, 15A, 15B, 15C, 16 or 18, and are cultured under suitable, expression-promoting conditions and the gene product is subsequently purified from the culture.

42. A DNA sequence as claimed in claim 28 or gene product which is encoded by a DNA sequence as claimed in claim 28 being a component of a drug.

43. The method of using a DNA sequence as claimed in claim 28 or of a gene product which is encoded by a DNA sequence as claimed in claim 28 for the treatment, for preparing a drug for the treatment, or for the treatment and for preparing a drug for the treatment of oncoses, chronic or acute states of hypoxia, cardiovascular disorders, (neuro)degenerative disorders, disorders of the immune system, in particular autoimmune disorders, neurological disorders.

44. The method of using as claimed in claim 43 treating stroke, multiple sclerosis, Parkinson's disease, amyotrophic lateral sclerosis, heredodegenerative ataxias, Huntington's disease, neuropathies and epilepsies.

45. A method for identifying pharmaceutically active compounds that modulate the function of an ee3 protein, wherein
(a) a suitable host cell system is transfected with an expression vector coding for the protein according to FIG. 13, 14, 15A, 15B, 15C, 16 or 18, and, where appropriate, at least one expression vector coding for at least one reporter gene, and
(b) a parameter suitable for observing the function mediated by a gene product according to the invention as claimed in claim 7, is measured for the host cell system obtained according to (a) in a suitable assay system after addition of a test compound, compared to the control without addition of a test compound.

46. A method as claimed in claim 45, wherein the parameter is suitable for observing cell conditions selected from the group consisting of apoptosis, cell growth, cell proliferation and cell plasticity.

47. A method as claimed in claim 45, wherein a further step (c) comprises determining the binding site of the pharmaceutically active compound on a protein of the invention by a suitable biochemical or structural-biological method.

48. A method as claimed in claim 45, wherein intracellular Ca release is measured a parameter according to (b) within an assay design.

49. The method of using of a compound that modulates the function of an ee3 protein for the treatment of diseases, for preparing a drug for the treatment of diseases, or for the treatment and for preparing a drug for the treatment of diseases in which chronic or acute states of hypoxia may occur or are involved selected from the group consisting of myocardial infarct, heart failure, cardiomyopathies, myocarditis, pericarditis, perimyocarditis, coronary heart disease, congenital heart defects with right-left shunt, tetralogy/pentalogy of Fallot, Eisenmenger syndrome, shock, hypoperfusions of extremities, arterial occlusive disease (AOD), peripheral AOD (pAOD), carotid stenosis, renal artery stenosis, small vessel disease, intracerebral bleeding, cerebral vein and sinus thromboses, vascular malformations, subarachnoidal hemorrhages, vascular dementia, Biswanger's disease, subcortical arteriosclerotic encephalopathy, multiple cortical infarcts during embolisms, vasculitis, diabetic retinopathy, consecutive symptoms of anemias of different causes, lung fibroses, emphysema, lung edema: ARDS, IRDS, recurring pulmonary embolisms, oncoses, disorders of the immune system, viral infectious diseases, bacterial infections, degenerative disorders, else neurological disorders, muscle relaxants, endocrinological disorders and dermatological disorders; control of chronic or acute states of pain, genetic diseases, disorders in the psychological field, wound healing, support of sexual function, cardiovascular disorders, increase in cerebral function, neurodegenerative disorders, muscular dystrophy, viral infectious diseases, oncoses and autoimmune disorders or cerebral ischemias.

50. A method for identifying a cellular interaction partner of an ee3 protein or derivative, using a “yeast two-hybrid” system.

51. The method of using a DNA sequence as claimed in claim 28 or of a gene product which is encoded by a DNA sequence as claimed in claim 28 for identifying further proteins involved in signal transduction mediated by ee3 protein.
